(19) United States
(12) Patent Application Publication
(10) Pub. No.: US 2022/0372024 A1
(43) Pub. Date: Nov. 24, 2022

(54) CRYSTALLINE FORMS OF ENTRECTINIB

(71) Applicant: Johnson Matthey Public Limited Company, London (GB)

(72) Inventors: Thierry BONNAUD, Billingham (GB); Zoe PRENTICE, Cambridge (US); Adam PATTERSON, Cambridge (GB)

(21) Appl. No.: 17/755,414

(22) PCT Filed: Oct. 30, 2020
(86) PCT No.: PCT/GB2020/052740

§ 371 Date: Apr. 28, 2022

(30) Foreign Application Priority Data
Nov. 1, 2019 (GB) 1915908.6
Feb. 14, 2020 (GB) 2002054.1
Jun. 17, 2020 (GB) 2009218.5

(51) Int. Cl. C07D 405/12

(52) U.S. Cl. CPC C07D 405/12; C07B 2200/13

(57) ABSTRACT

The present invention relates to crystalline forms of entrectinib, processes for their preparation, and to pharmaceutical compositions containing the crystalline forms.

The present invention relates to crystalline forms of entrectinib, processes for their preparation, and to pharmaceutical compositions containing the crystalline forms.

### BACKGROUND

Entrectinib has the IUPAC name of N-[5-[(3,5-difluorophenyl)methyl]-1H-indazol-3-yl]-4-(4-methylpiperazin-1-yl)-2-(oxan-4-ylamino)benzamide and the chemical structure illustrated below:

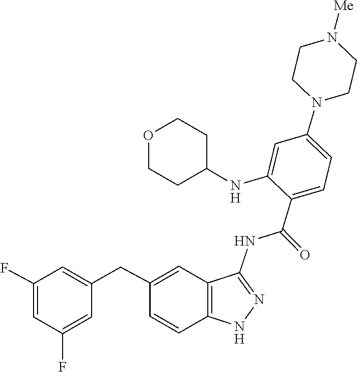 [SMILES: CN1CCN(c2ccc(C(=O)Nc3n[nH]c4ccc(Cc5cc(F)cc(F)c5)cc34)c(NC3CCOCC3)c2)CC1]

In the EU, an orphan designation has been granted for entrectinib for the treatment of neuroblastoma.

U.S. Pat. Nos. 8,299,057, 9,029,356 and 9,085,565 (all to Nerviano Medical Sciences S.R.L.) describe entrectinib and its preparation.

Information about the solid-state properties of a drug substance is important. For example, different forms may have differing solubilities. Also, the handling and stability of a drug substance may depend on the solid form.

Polymorphism may be defined as the ability of a compound to crystallise in more than one distinct crystal species and different crystal arrangements of the same chemical composition are termed polymorphs. Polymorphs of the same compound arise due to differences in the internal arrangement of atoms and have different free energies and therefore different physical properties such as solubility, chemical stability, melting point, density, flow properties, hygroscopicity, bioavailability, and so forth. The compound entrectinib may exist in a number of polymorphic forms and many of these forms may be undesirable for producing pharmaceutically acceptable compositions. This may be for a variety of reasons including lack of stability, high hygroscopicity, low aqueous solubility and difficulty in handing.

### Definitions

The term “about” or “approximately” means an acceptable error for a particular value as determined by a person of ordinary skill in the art, which depends in part on how the value is measured or determined.

In certain embodiments, the term “about” or “approximately” means within 1, 2, 3 or 4 standard deviations. In certain embodiments, the term “about” or “approximately” means within 30%, 25%, 20%, 15%, 10%, 9%, 8%, 7%, 6%, 5%, 4%, 3%, 2%, 1%, or 0.5% of a given value or range. In certain embodiments and with reference to X-ray powder diffraction two-theta peaks, the terms “about” or “approximately” means within ±0.2° 2θ.

The term “ambient temperature” means one or more room temperatures between about 15° C. to about 30° C., such as about 15° C. to about 25° C.

The term “anti-solvent” refers to a first solvent which is added to a second solvent to reduce the solubility of a compound in that second solvent. The solubility may be reduced sufficiently such that precipitation of the compound from the first and second solvent combination occurs.

The term “consisting” is closed and excludes additional, unrecited elements or method steps in the claimed invention.

The term “consisting essentially of” is semi-closed and occupies a middle ground between “consisting” and “comprising”. “Consisting essentially of” does not exclude additional, unrecited elements or method steps which do not materially affect the essential characteristic(s) of the claimed invention.

The term “comprising” is inclusive or open-ended and does not exclude additional, unrecited elements or method steps in the claimed invention. The term is synonymous with “including but not limited to”.

The term “comprising” encompasses three alternatives, namely (i) “comprising”, (ii) “consisting”, and (iii) “consisting essentially of”.

The term “crystalline” and related terms used herein, when used to describe a compound, substance, modification, material, component or product, unless otherwise specified, means that the compound, substance, modification, material, component or product is substantially crystalline as determined by X-ray diffraction. See, e.g., Remington: The Science and Practice of Pharmacy, 21st edition, Lippincott, Williams and Wilkins, Baltimore, Md. (2005); The United States Pharmacopeia, 23rd ed., 1843-1844 (1995).

The terms “polymorph,” “polymorphic form” or related term herein, refer to a crystal form of one or more molecules of entrectinib, or entrectinib molecular complex thereof that can exist in two or more forms, as a result different arrangements or conformations of the molecule(s) in the crystal lattice of the polymorph.

The term “pharmaceutical composition” is intended to encompass a pharmaceutically effective amount of entrectinib of the invention and a pharmaceutically acceptable excipient. As used herein, the term “pharmaceutical compositions” includes pharmaceutical compositions such as tablets, pills, powders, liquids, suspensions, emulsions, granules, capsules, suppositories, or injection preparations.

The term “excipient” refers to a pharmaceutically acceptable organic or inorganic carrier substance. Excipients may be natural or synthetic substances formulated alongside the active ingredient of a medication, included for the purpose of bulking-up formulations that contain potent active ingredients (thus often referred to as “bulking agents,” “fillers,” or “diluents”), or to confer a therapeutic enhancement on the active ingredient in the final dosage form, such as facilitating drug absorption or solubility. Excipients can also be useful in the manufacturing process, to aid in the handling of the active substance, such as by facilitating powder flowability or non-stick properties, in addition to aiding in vitro stability such as prevention of denaturation over the expected shelf life.

The term “patient” refers to an animal, preferably a patient, most preferably a human, who has been the object of treatment, observation or experiment. Preferably, the patient has experienced and/or exhibited at least one symptom of the disease or disorder to be treated and/or prevented. Further, a patient may not have exhibited any symptoms of the disorder, disease or condition to be treated and/prevented, but has been deemed by a physician, clinician or other medical professional to be at risk for developing said disorder, disease or condition.

The terms “treat,” “treating” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more therapeutic agents to a patient with such a disease or disorder. In some embodiments, the terms refer to the administration of a molecular complex provided herein, with or without other additional active agents, after the onset of symptoms of a disease.

The term “overnight” refers to the period of time between the end of one working day to the subsequent working day in which a time frame of about 12 to about 18 hours has elapsed between the end of one procedural step and the instigation of the following step in a procedure.

### DESCRIPTION OF THE FIGURES

FIG. 1 is a representative XRPD pattern of entrectinib anhydrate (Form A).

FIG. 2 is a representative TGA thermogram and a DSC thermogram of entrectinib anhydrate (Form A).

FIG. 3 is a representative GVS isotherm plot of entrectinib anhydrate (Form A). The solid black diamond symbol (-●-) represents the cycle 1 sorption isotherm plot. The solid grey symbol (-▪-) represents the cycle 1 desorption isotherm plot. The solid grey triangle symbol (-▴-) represents the cycle 2 sorption isotherm plot. The black cross symbol ( [symbol] ) represents the cycle 2 desorption isotherm plot. The grey star-like symbol ( [symbol] ) represents the cycle 3 sorption isotherm plot.

FIG. 4 is a representative XRPD pattern overlay of entrectinib anhydrate (Form A) before (bottom) and after (top) the GVS experiment.

FIG. 5 is a representative XRPD overlay of entrectinib anhydrate (Form A) before storage (bottom), entrectinib anhydrate (Form A) after storage at 40° C./75% RH (relative humidity) for 28 days (middle), and entrectinib anhydrate (Form A) after storage at 25° C./97% RH for 28 days (top).

FIG. 6 is a representative XRPD pattern of entrectinib THF solvate (Form B).

FIG. 7 is a representative TGA thermogram and DSC thermogram of entrectinib THF solvate (Form B).

FIG. 8 is a representative XRPD pattern of entrectinib anhydrate (Form C).

FIG. 9 is a representative TGA thermogram and DSC thermogram of entrectinib anhydrate (Form C).

FIG. 10 is a representative GVS isotherm plot of entrectinib anhydrate (Form C). The solid black diamond symbol ( [symbol] ) represents the cycle 1 sorption isotherm plot. The solid grey symbol ( [symbol] ) represents the cycle 1 desorption isotherm plot. The solid grey triangle symbol ( [symbol] ) represents the cycle 2 sorption isotherm plot. The black cross symbol ( [symbol] ) represents the cycle 2 desorption isotherm plot. The grey star-like symbol ( [symbol] ) represents the cycle 3 sorption isotherm plot.

FIG. 11 is a representative XRPD pattern overlay of entrectinib anhydrate (Form C) before (bottom) and after (top) the GVS experiment.

FIG. 12 is a representative XRPD overlay of entrectinib anhydrate (Form C) before storage (bottom), entrectinib anhydrate (Form C) after storage at 40° C./75% for 28 days (middle), and entrectinib anhydrate (Form C) after storage at 25° C./97% RH for 28 days (top).

FIG. 13 is a representative XRPD pattern of entrectinib nitromethane solvate (Form D).

FIG. 14 is a representative TGA thermogram and DSC thermogram of entrectinib nitromethane solvate (Form D).

FIG. 15 is a representative XRPD pattern of entrectinib acetonitrile solvate (Form E).

FIG. 16 is a representative TGA thermogram and DSC thermogram of entrectinib acetonitrile solvate (Form E).

FIG. 17 is a representative XRPD pattern of entrectinib isopropyl acetate solvate (Form F).

FIG. 18 is a representative TGA thermogram and DSC thermogram of entrectinib isopropyl acetate solvate (Form F).

FIG. 19 is a representative XRPD pattern of entrectinib monohydrate (Form G).

FIG. 20 is a representative TGA thermogram and DSC thermogram of entrectinib monohydrate (Form G).

FIG. 21 is a representative GVS isotherm plot of entrectinib monohydrate (Form G).

FIG. 22 is a representative XRPD pattern overlay of entrectinib monohydrate (Form G) before (bottom) and after (top) the GVS experiment.

FIG. 23 is a representative XRPD overlay of entrectinib monohydrate (Form G) before storage (bottom), entrectinib monohydrate (Form G) after storage at 40° C./75% for 28 days (middle), and entrectinib monohydrate (Form G) after storage at 25° C./97% RH for 28 days (top).

FIG. 24 is a representative XRPD pattern of entrectinib 2-methyl tetrahydrofuran (2-Me THF) solvate (Form H).

FIG. 25 is a representative TGA thermogram and DSC thermogram of entrectinib 2-methyl tetrahydrofuran (2-Me THF) solvate (Form H).

FIG. 26 is a representative XRPD pattern of entrectinib methanol solvate (Form J).

FIG. 27 is a representative TGA thermogram and DSC thermogram of entrectinib methanol solvate (Form J).

FIG. 28 is a representative IDR dissolution profile at pH 2.8 for entrectinib anhydrate (Form A), entrectinib anhydrate (Form C) and entrectinib monohydrate (Form G).

### DESCRIPTION OF THE INVENTION

### Entrectinib Anhydrate

It has been discovered that entrectinib can be prepared in well-defined and consistently reproducible anhydrous crystalline forms. Moreover, reliable and scalable methods for producing these anhydrous crystalline forms have been developed. The entrectinib polymorphs provided by the present invention are useful as an active ingredient in pharmaceutical formulations. In certain embodiments, the anhydrous crystalline forms are purifiable. In certain embodiments and depending on time, temperature and humidity, the anhydrous crystalline forms are stable. In certain embodiments, the anhydrous crystalline forms are easy to isolate and handle. In certain embodiments, the processes for preparing the anhydrous crystalline forms are scalable.

The crystalline forms described herein may be characterised using a number of methods known to the skilled person in the art, including single crystal X-ray diffraction, X-ray powder diffraction (XRPD), differential scanning calorimetry (DSC), thermal gravimetric analysis (TGA), infrared spectroscopy, Raman spectroscopy, nuclear magnetic resonance (NMR) spectroscopy (including solution and solid-state NMR). The chemical purity may be determined by standard analytical methods, such as thin layer chromatography (TLC), gas chromatography, high performance liquid chromatography (HPLC), and mass spectrometry (MS).

### Entrectinib Anhydrate Form A

In one aspect, the present invention provides a crystalline form of entrectinib which is crystalline entrectinib anhydrate (designated herein as Form A).

Crystalline entrectinib anhydrate Form A may be free or substantially free of other polymorphic forms of entrectinib. In certain embodiments, the polymorphic purity of the anhydrate is ≥90%, ≥91%, ≥92%, ≥93%, ≥94%, ≥95% or higher. In certain embodiments, the polymorphic purity of the anhydrate is ≥95%. In certain embodiments, the polymorphic purity of the anhydrate is ≥96%. In certain embodiments, the polymorphic purity of the anhydrate is ≥97%. In certain embodiments, the polymorphic purity of the anhydrate is ≥98%. In certain embodiments, the polymorphic purity of the anhydrate is ≥99%.

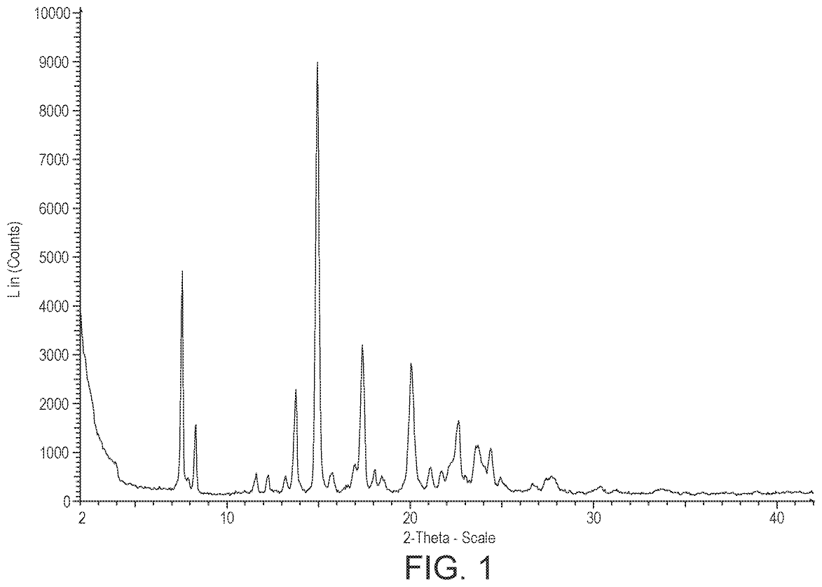

The anhydrate may have an X-ray powder diffraction pattern comprising one or more peaks (for example 1, 2, 3, 4, 5, 6, 7, 8, 9, or 10 peaks) selected from the group consisting of about 7.5, 7.8, 8.3, 11.0, 11.6, 12.2, 13.2, 13.7, 14.9, 15.4, 15.8, 16.5, 17.0, 17.4, 18.1, 18.5, 19.6, 20.1, 21.1, 21.7, 22.1, 22.6, 23.2, 23.7, 24.1, 24.4, 25.0, 26.8, 27.4, 27.8, and 28.0 degrees two-theta ±0.2 degrees two-theta. In one embodiment, the anhydrate may have the X-ray powder diffraction pattern substantially as shown in FIG. 1.

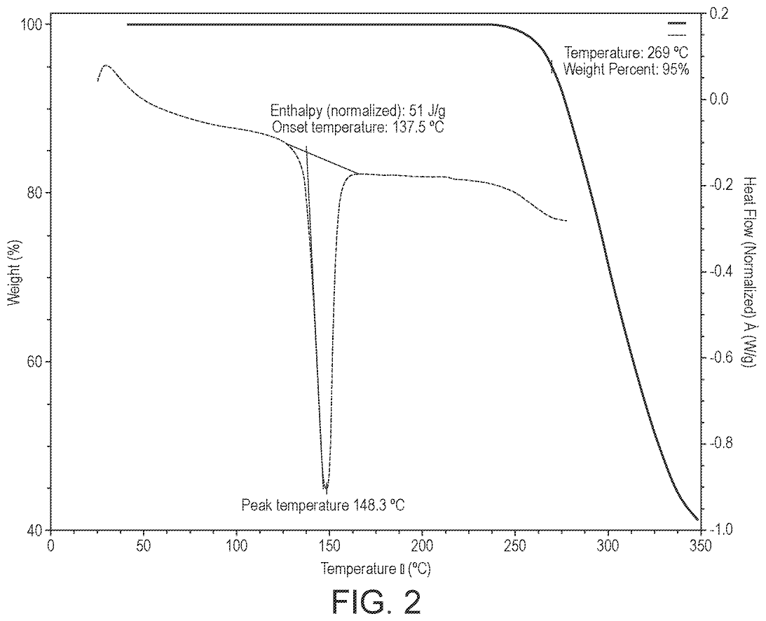

The anhydrate may have a DSC thermogram comprising an endothermic event with an onset at about 137.5° C. In one embodiment, the anhydrate may have a DSC thermogram substantially as shown in FIG. 2.

The anhydrate may have a TGA thermogram comprising no substantially mass loss when heated from about ambient temperature to about 200° C. In one embodiment, the anhydrate may have a TGA thermogram substantially as shown in FIG. 2.

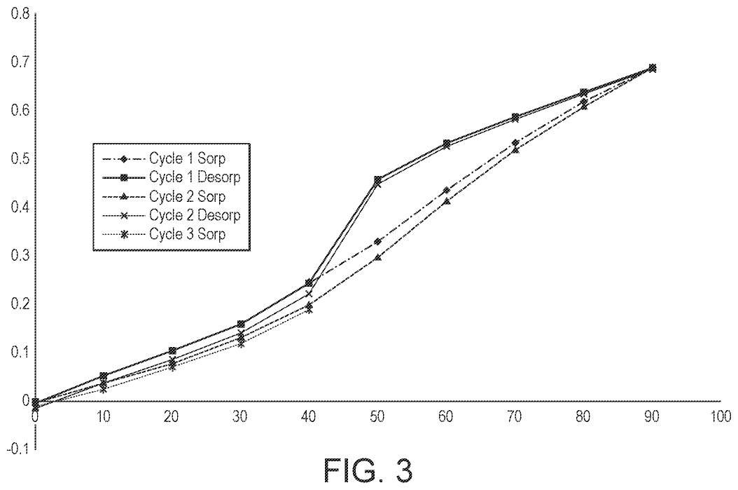

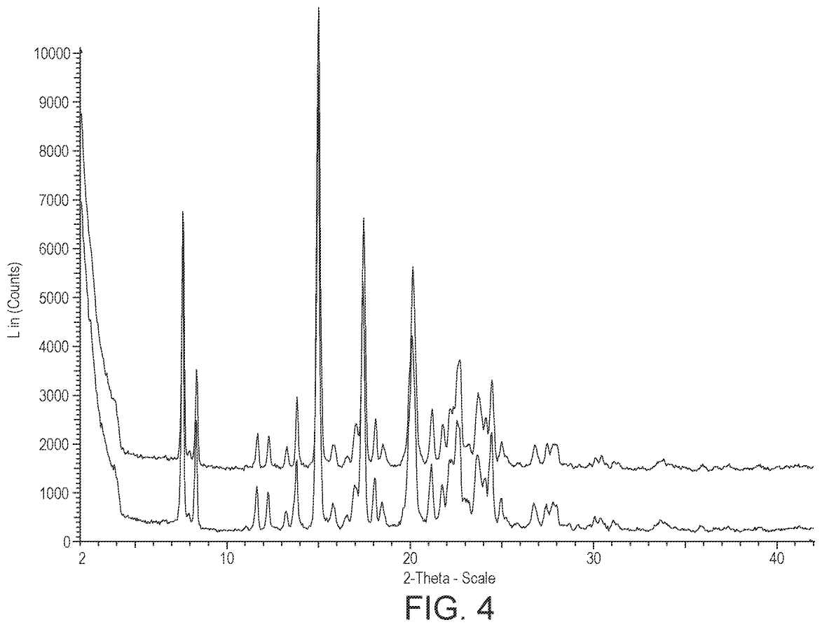

The anhydrate may have a GVS isotherm plot substantially as shown in FIG. 3. The XRPD after analysis showed that the crystalline entrectinib anhydrate was unchanged i.e. there was no change in form after GVS (see FIG. 4).

The crystalline entrectinib anhydrate Form A described above may be prepared by a process comprising the steps of:
(a) dissolving entrectinib in a first solvent selected from the group consisting of methyl ethyl ketone (MEK), propanol (e.g. 1- or 2-propanol), and combinations thereof;
(b) adding acetonitrile to the solution of entrectinib; and
(c) recovering entrectinib anhydrate Form A as a crystalline solid.

The quantity of the first solvent is not particularly limiting provided there is enough solvent to dissolve the entrectinib and form a solution. The w/v ratio of entrectinib to the first solvent may be in the range of about 1 mg of entrectinib:about 1 to about 100 μl of solvent, such as about 1 mg of entrectinib:about 1 to about 50 μl of solvent, for example about 1 mg of entrectinib:about 1 to about 20 μl of solvent.

The entrectinib may be dissolved in the first solvent at ambient temperature or less. In one embodiment, the dissolving step may be carried out at one or more temperatures in the range of ≥about ≥0° C. to about ≤25° C. In some embodiments, the dissolving step is carried out at one or more temperatures ≥about 1° C. In some embodiments, the dissolving step is carried out at one or more temperatures ≥about 2° C. In some embodiments, the dissolving step is carried out at one or more temperatures ≥about 3° C. In some embodiments, the dissolving step is carried out at one or more temperatures ≥about 4° C. In some embodiments, the dissolving step is carried out at one or more temperatures about 5° C. In some embodiments, the dissolving step is carried out at one or more temperatures ≤about 20° C. In some embodiments, the dissolving step is carried out at one or more temperatures ≤about 15° C. In some embodiments, the dissolving step is carried out at one or more temperatures ≤about 10° C. In one embodiment, the dissolving step is carried out at one or more temperatures in the range of ≥about 0° C. to ≤about 10° C., for example, about 5° C. In one embodiment, the dissolving step may be carried out at about ambient temperature e.g. about 25° C.

Alternatively, the entrectinib may be dissolved in the solvent at a temperature greater than ambient i.e. greater than 30° C. and below the boiling point of the reaction mixture. The boiling point of the reaction mixture may vary depending on the pressure under which the contacting step is conducted. In one embodiment, the dissolving step is carried out at atmospheric pressure (i.e. 1.0135×10⁵ Pa). In one embodiment, the dissolving step may be carried out at one or more temperatures in the range of ≥about 40° C. to about ≤60° C. In some embodiments, the dissolving step is carried out at one or more temperatures ≥about 41° C. In some embodiments, the dissolving step is carried out at one or more temperatures ≥about 42° C. In some embodiments, the dissolving step is carried out at one or more temperatures ≥about 43° C. In some embodiments, the dissolving step is carried out at one or more temperatures ≥about 44° C. In some embodiments, the dissolving step is carried out at one or more temperatures ≥about 45° C. In some embodiments, the dissolving step is carried out at one or more temperatures ≥≥about 46° C. In some embodiments, the dissolving step is carried out at one or more temperatures ≥about 47° C. In some embodiments, the dissolving step is carried out at one or more temperatures ≥about 48° C. In some embodiments, the dissolving step is carried out at one or more temperatures ≥about 49° C. In some embodiments, the dissolving step is carried out at one or more temperatures ≥about 50° C. In some embodiments, the dissolving step is carried out at one or more temperatures ≤about 59° C. In some embodiments, the dissolving step is carried out at one or more temperatures ≤about 58° C. In some embodiments, the dissolving step is carried out at one or more temperatures ≤about 57° C. In some embodiments, the dissolving step is carried out at one or more temperatures ≤about 56° C. In some embodiments, the dissolving step is carried out at one or more temperatures ≤about 55° C. In some embodiments, the dissolving step is carried out at one or more temperatures ≤about 54° C. In some embodiments, the dissolving step is carried out at one or more temperatures ≤about 53° C. In some embodiments, the dissolving step is carried out at one or more temperatures ≤about 52° C. In some embodiments, the dissolving step is carried out at one or more temperatures ≤about 51° C. In one embodiment, the dissolving step is carried out at one or more temperatures in the range of ≥about 45° C. to ≤about 55° C. In one embodiment, the dissolving step is carried out at a temperature of about 50° C.

The dissolution of entrectinib may be encouraged through the use of an aid such as stirring, shaking and/or sonication. Additional solvent may be added to aid the dissolution of the entrectinib.

The period of time for which the mixture of entrectinib and solvent is treated at the desired temperature is not particularly limiting. In one embodiment, the period of time may be from about 1 minute to about 24 hours, for example, about 5 minutes.

In step (b), acetonitrile is added to the reaction mixture. The quantity of acetonitrile is not particularly limiting. The w/v ratio of entrectinib to acetonitrile may be in the range of about 1 mg of entrectinib:about 1 to about 2000 μl of acetonitrile, such as about 1 mg of entrectinib:about 100 to about 1500 μl of acetonitrile, for example about 1 mg of entrectinib:about 750 to about 1500 μl of acetonitrile, e.g. about 1 mg of entrectinib:about 1000 μl of acetonitrile. These w/v ratios have been calculated using the mass of entrectinib initially dissolved in the first solvent i.e. the quantity of entrectinib inputted into the process.

After the addition of the acetonitrile, the reaction mixture may be treated for a period of time at ambient temperature or less as described above in connection with first solvent.

Alternatively, the reaction mixture may be treated in the solvent mixture for a period of time at a temperature greater than ambient i.e. greater than 30° C. and below the boiling point of the reaction mixture as described above in connection with the first solvent.

The reaction mixture may then be treated for a further period of time e.g. about 1 minute to about 24 hours, such as 1 hour.

The solution or suspension may then be cooled such that the resulting solution or suspension has a temperature below that of the solution or suspension of step (b). The rate of cooling may be from about 0.05° C./minute to about 2° C./minute, such as about 0.1° C./minute to about 1.5° C./minute, for example about 0.1° C./minute. When a solution of entrectinib is cooled, a suspension may eventually be observed.

The solution may be cooled to ambient temperature or a temperature of less than ambient temperature. In one embodiment, the solution or suspension may be cooled to one or more temperatures in the range of ≥about 0° C. to about ≤20° C. In some embodiments, the solution is cooled to one or more temperatures ≥about 1° C. In some embodiments, the solution is cooled to one or more temperatures ≥about 2° C. In some embodiments, the solution is cooled to one or more temperatures ≥about 3° C. In some embodiments, the solution is cooled to one or more temperatures ≥about 4° C. In some embodiments, the solution is cooled to one or more temperatures ≥about 5° C. In some embodiments, the solution is cooled to one or more temperatures ≤about 15° C. In some embodiments, the solution is cooled to one or more temperatures ≤about 14° C. In some embodiments, the solution is cooled to one or more temperatures ≤about 13° C. In some embodiments, the solution is cooled to one or more temperatures ≤about 12° C. In some embodiments, the solution may be cooled to one or more temperatures ≤about 11° C. In some embodiments, the solution is cooled to one or more temperatures ≤about 10° C. In one embodiment, the solution is cooled to one or more temperatures in the range of about 5° C. to about 10° C.

In step (c), the entrectinib anhydrate is recovered as a crystalline solid. The crystalline anhydrate may be recovered by directly by filtering, decanting or centrifuging. If desired, the suspension may be mobilised with additional portions of the solvent prior to recovery of the crystalline solid. Alternatively, a proportion or substantially all of the solvent may be evaporated prior to recovery of the crystalline solid.

Howsoever the crystalline anhydrate is recovered, the separated anhydrate may be washed with solvent (e.g. one or more of the solvents described above) and dried. Drying may be performed using known methods, for example, at temperatures in the range of about 10° C. to about 60° C., such as about 20° C. to about 40° C., for example, ambient temperature under vacuum (for example about 1 mbar to about 30 mbar) for about 1 hour to about 24 hours. Alternatively, the crystalline anhydrate may be left to dry under ambient temperature naturally i.e. without the active application of vacuum. It is preferred that the drying conditions are maintained below the point at which the anhydrate degrades and so when the anhydrate is known to degrade within the temperature or pressure ranges given above, the drying conditions should be maintained below the degradation temperature or vacuum.

Steps (a) to (c) may be carried out one or more times (e.g. 1, 2, 3, 4 or 5 times). When steps (a) to (c) are carried out more than once (e.g. 2, 3, 4 or 5 times), step (a) may be optionally seeded with crystalline entrectinib anhydrate Form A which was previously prepared and isolated by a method described herein).

Alternatively or in addition, when steps (a) to (c) are carried out more than once (e.g. 2, 3, 4 or 5 times), the solution or suspension formed in step (b) may be optionally seeded with crystalline entrectinib anhydrate Form A (which was previously prepared and isolated by a method described herein).

The crystalline entrectinib anhydrate Form A described above may be prepared by a process comprising the step of exposing a crystalline entrectinib solvate to water vapour, wherein the crystalline entrectinib solvate is selected from the group consisting of entrectinib acetonitrile solvate Form E, entrectinib THF solvate Form B, and entrectinib nitromethane solvate Form D.

The entrectinib solvate may be placed within an enclosed chamber under vacuum and water vapour (for instance, in the form of moist air or moist nitrogen with a relative humidity (RH) of e.g. about 75% or about 97%) may be bled into the enclosed chamber at a temperature from about ambient temperature to about 50° C., for example, about 25° C., or about 40° C. Alternatively, the entrectinib solvate may be exposed to a moist atmosphere at approximately atmospheric pressure within an enclosed chamber containing a source of liquid water at a temperature from about ambient temperature to about 50° C., for example, about 25° C., or about 40° C.

Crystalline entrectinib anhydrate Form A is produced by the described process even though water is present. Water is not incorporated into the entrectinib crystals being formed.

The period of time for which the crystalline entrectinib solvate is exposing to water vapour at the desired temperature is not particularly limiting. In one embodiment, the period of time may be from about 1 minute to about 10 days, for example, about 7 days.

In another aspect, crystalline entrectinib anhydrate Form A may be prepared by a process comprising the step of treating a crystalline entrectinib solvate under vacuum,

wherein the crystalline entrectinib solvate is selected from the group consisting of entrectinib acetonitrile solvate Form E, entrectinib THF solvate Form B, and entrectinib nitromethane solvate Form D.

The entrectinib solvate may be placed within an enclosed chamber under vacuum at a temperature from about ambient temperature to about 50° C., for example, about 25° C., or about 40° C.

The period of time for which the crystalline entrectinib solvate is treated under vacuum at the desired temperature is not particularly limiting. In one embodiment, the period of time may be from about 1 minute to about 7 days, for example, about 3 days.

The crystalline entrectinib anhydrate Form A formed by the processes described above may be free or substantially free of other polymorphic forms of entrectinib. In certain embodiments, the polymorphic purity of the anhydrate is ≥90%, ≥91%, ≥92%, ≥93%, ≥94%, ≥95% or higher. In certain embodiments, the polymorphic purity of the anhydrate is ≥95%. In certain embodiments, the polymorphic purity of the anhydrate is ≥96%. In certain embodiments, the polymorphic purity of the anhydrate is ≥97%. In certain embodiments, the polymorphic purity of the anhydrate is ≥98%. In certain embodiments, the polymorphic purity of the anhydrate is ≥99%.

In another aspect, the present invention relates to a pharmaceutical composition comprising crystalline entrectinib anhydrate Form A as described herein and a pharmaceutically acceptable excipient.

In another aspect, the present invention relates to a method for treating cancer in a patient comprising administering a therapeutically effective amount of crystalline entrectinib anhydrate Form A as described herein to the patient. The method of treatment includes the treatment of neuroblastoma.

In another aspect, the present invention relates to crystalline entrectinib anhydrate Form A as described herein for use in treating cancer, such as the treatment of neuroblastoma.

### Entrectinib Anhydrate Form C

In one aspect, the present invention provides a crystalline form of entrectinib which is crystalline entrectinib anhydrate (designated herein as Form C).

Crystalline entrectinib anhydrate Form C may be free or substantially free of other polymorphic forms of entrectinib. In certain embodiments, the polymorphic purity of the anhydrate is ≥90%, ≥91%, ≥92%, ≥93%, ≥94%, ≥95% or higher. In certain embodiments, the polymorphic purity of the anhydrate is ≥95%. In certain embodiments, the polymorphic purity of the anhydrate is ≥96%. In certain embodiments, the polymorphic purity of the anhydrate is ≥97%. In certain embodiments, the polymorphic purity of the anhydrate is ≥98%. In certain embodiments, the polymorphic purity of the anhydrate is ≥99%.

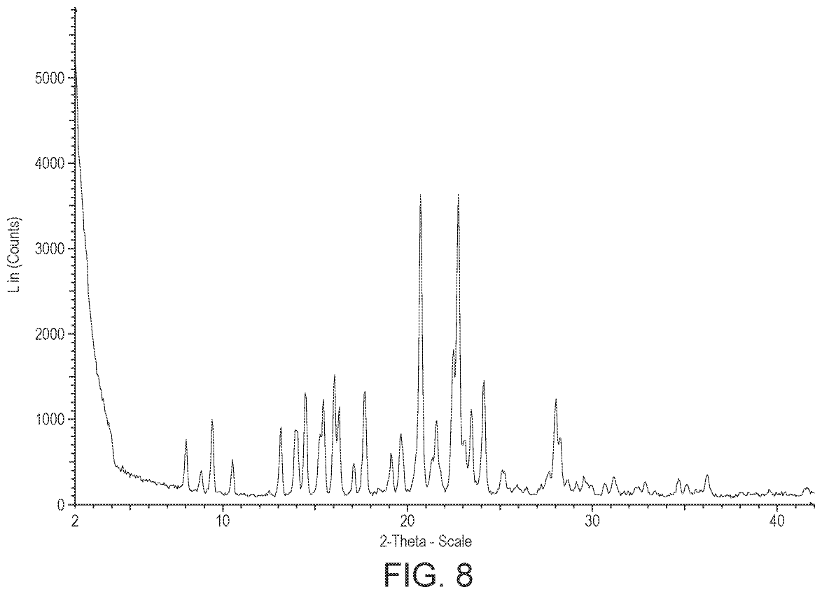

The anhydrate may have an X-ray powder diffraction pattern comprising one or more peaks (for example 1, 2, 3, 4, 5, 6, 7, 8, 9, or 10 peaks) selected from the group consisting of about 8.0, 8.8, 9.4, 10.5, 13.1, 14.0, 14.5, 15.2, 15.4, 16.0, 16.2, 17.1, 17.7, 19.1, 19.6, 20.7, 21.3, 21.6, 22.4, 22.7, 23.1, 23.5, 24.1, 25.2, 25.9, 26.4, 27.3, 27.7, 28.0, 28.3, 28.6, 29.2, 29.5, and 29.9 degrees two-theta ±0.2 degrees two-theta. In one embodiment, the anhydrate may have the X-ray powder diffraction pattern substantially as shown in FIG. 8.

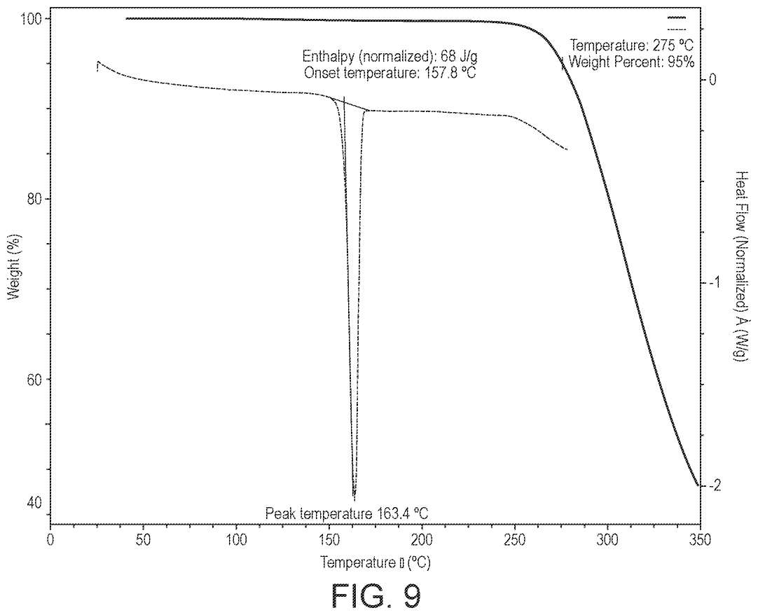

The anhydrate may have a DSC thermogram comprising an endothermic event with an onset at about 157.8° C. In one embodiment, the anhydrate may have a DSC thermogram substantially as shown in FIG. 9.

The anhydrate may have a TGA thermogram comprising no substantially mass loss when heated from about ambient temperature to about 200° C. In one embodiment, the anhydrate may have a TGA thermogram substantially as shown in FIG. 9.

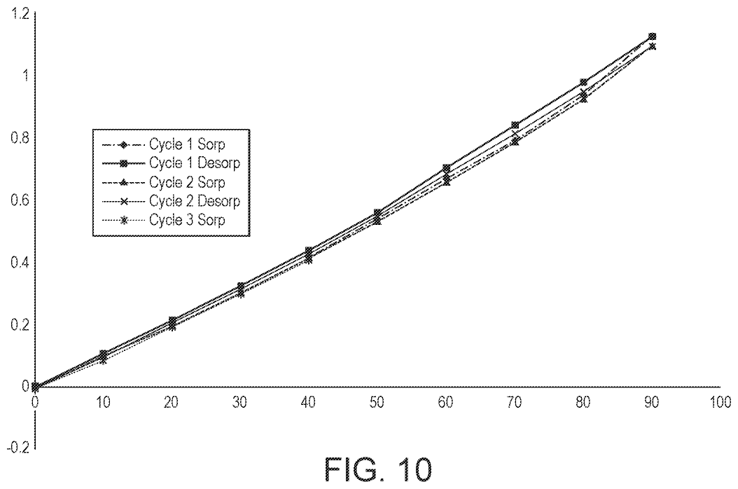

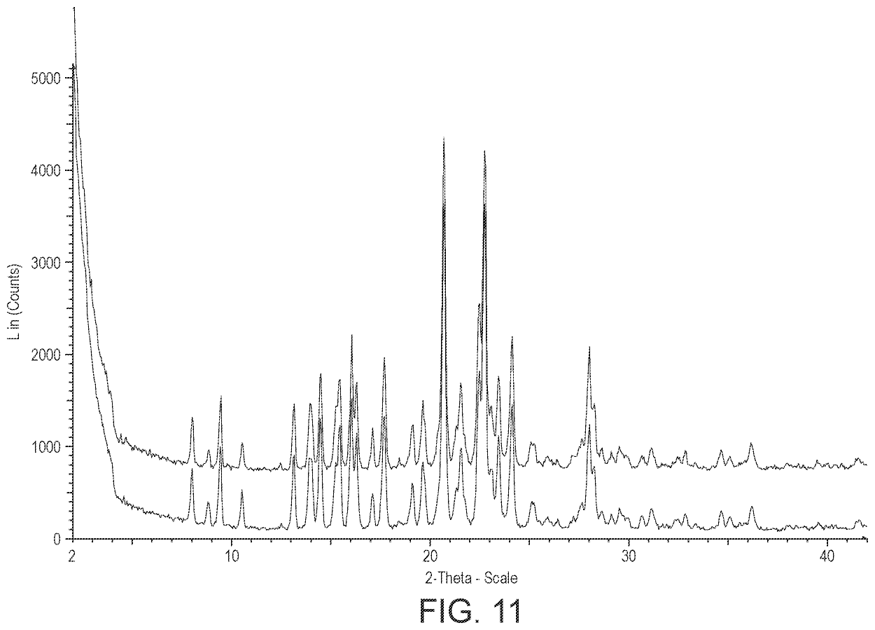

The anhydrate may have a GVS isotherm plot substantially as shown in FIG. 10. The XRPD after analysis showed that the crystalline entrectinib anhydrate was unchanged i.e. there was no change in form after GVS (see FIG. 11).

The crystalline entrectinib anhydrate Form C described above may be prepared by a process comprising the steps of:
(a) contacting entrectinib with a solvent selected from the group consisting of acetone, methyl ethyl ketone, methyl isobutyl ketone, dichloromethane, 1,2-dimethoxyethane, water, ethyl acetate, 1,2-dimethoxyethanol, and combinations thereof;
(b) forming a solution or suspension of entrectinib in the solvent; and
(c) recovering entrectinib anhydrate Form C as a crystalline solid.

In one embodiment, the solvent is a combination of acetone and water. The v/v ratio of acetone:water may be about 90:about 10 or about 95:about 5.

The quantity of solvent is not particularly limiting provided there is enough solvent to dissolve the entrectinib and form a solution, or suspend the entrectinib. The w/v ratio of entrectinib to solvent may be in the range of about 1 mg of entrectinib:about 1 to about 1000 μl of solvent, such as about 1 mg of entrectinib:about 1 to about 500 μl of solvent, for example about 1 mg of entrectinib:about 1 to about 150 μl of solvent, e.g. about 1 mg of entrectinib:about 5 to about 110 μl of solvent.

The entrectinib may be contacted with the solvent at ambient temperature or less. In one embodiment, the contacting step may be carried out at one or more temperatures in the range of ≥about 0° C. to about ≤25° C. In some embodiments, the contacting step is carried out at one or more temperatures ≥about 1° C. In some embodiments, the contacting step is carried out at one or more temperatures ≥about 2° C. In some embodiments, the contacting step is carried out at one or more temperatures ≥about 3° C. In some embodiments, the contacting step is carried out at one or more temperatures ≥about 4° C. In some embodiments, the contacting step is carried out at one or more temperatures ≥about 5° C. In some embodiments, the contacting step is carried out at one or more temperatures ≤about 20° C. In some embodiments, the contacting step is carried out at one or more temperatures ≤about 15° C. In some embodiments, the contacting step is carried out at one or more temperatures ≤about 10° C. In one embodiment, the contacting step is carried out at one or more temperatures in the range of ≥about 0° C. to ≤about 10° C., for example, about 5° C. In one embodiment, the contacting step may be carried out at ambient temperature e.g. about 25° C.

Alternatively, the entrectinib may be contacted with the solvent at a temperature greater than ambient i.e. greater than 30° C. and below the boiling point of the reaction mixture. The boiling point of the reaction mixture may vary depending on the pressure under which the contacting step is conducted. In one embodiment, the contacting step is carried out at atmospheric pressure (i.e. 1.0135×10⁵ Pa). In one embodiment, the contacting step may be carried out at one or more temperatures in the range of about 40° C. to about ≤60° C. In some embodiments, the contacting step is carried out at one or more temperatures ≥about 41° C. In some embodiments, the contacting step is carried out at one or more temperatures ≥about 42° C. In some embodiments, the contacting step is carried out at one or more temperatures ≥about 43° C. In some embodiments, the contacting step is carried out at one or more temperatures ≥about 44° C. In some embodiments, the contacting step is carried out at one or more temperatures ≥about 45° C. In some embodiments, the contacting step is carried out at one or more temperatures ≥about 46° C. In some embodiments, the contacting step is carried out at one or more temperatures ≥about 47° C. In some embodiments, the contacting step is carried out at one or more temperatures ≥about 48° C. In some embodiments, the contacting step is carried out at one or more temperatures ≥about 49° C. In some embodiments, the contacting step is carried out at one or more temperatures about 50° C. In some embodiments, the contacting step is carried out at one or more temperatures ≤about 59° C. In some embodiments, the contacting step is carried out at one or more temperatures ≤about 58° C. In some embodiments, the contacting step is carried out at one or more temperatures ≤about 57° C. In some embodiments, the contacting step is carried out at one or more temperatures ≤about 56° C. In some embodiments, the contacting step is carried out at one or more temperatures ≤about 55° C. In some embodiments, the contacting step is carried out at one or more temperatures ≤about 54° C. In some embodiments, the contacting step is carried out at one or more temperatures ≤about 53° C. In some embodiments, the contacting step is carried out at one or more temperatures ≤about 52° C. In some embodiments, the contacting step is carried out at one or more temperatures ≤about 51° C. In one embodiment, the contacting step is carried out at one or more temperatures in the range of ≥about 45° C. to ≤about 55° C. In one embodiment, the contacting step is carried out at a temperature of ≥about 50° C.

The dissolution or suspension of entrectinib may be encouraged through the use of an aid such as stirring, shaking and/or sonication. Additional solvent may be added to aid the dissolution or suspension of the entrectinib.

The period of time for which the mixture of entrectinib and solvent is treated at the desired temperature is not particularly limiting. In one embodiment, the period of time may be from about 1 minute to about 7 days, for example, about 5 minutes to about 3 days.

The solution or suspension may then be cooled such that the resulting solution or suspension has a temperature below that of the solution or suspension of step (b). The rate of cooling may be from about 0.05° C./minute to about 2° C./minute, such as about 0.1° C./minute to about 1.5° C./minute, for example about 0.1° C./minute. When a solution of entrectinib is cooled, a suspension may eventually be observed. When a suspension of entrectinib is cooled, no perceptible change in the appearance of the suspension may occur.

The solution or suspension may be cooled to ambient temperature or a temperature of less than ambient temperature. In one embodiment, the solution or suspension may be cooled to one or more temperatures in the range of ≥about 0° C. to about ≤20° C. In some embodiments, the solution or suspension is cooled to one or more temperatures ≥about 1° C. In some embodiments, the solution or suspension is cooled to one or more temperatures ≥about 2° C. In some embodiments, the solution or suspension is cooled to one or more temperatures ≥about 3° C. In some embodiments, the solution or suspension is cooled to one or more temperatures ≥about 4° C. In some embodiments, the solution or suspension is cooled to one or more temperatures ≥about 5° C. In some embodiments, the solution or suspension is cooled to one or more temperatures ≤about 15° C. In some embodiments, the solution or suspension is cooled to one or more temperatures ≤about 14° C. In some embodiments, the solution or suspension is cooled to one or more temperatures ≤about 13° C. In some embodiments, the solution or suspension is cooled to one or more temperatures ≤about 12° C. In some embodiments, the solution or suspension may be cooled to one or more temperatures ≤about 11° C. In some embodiments, the solution or suspension is cooled to one or more temperatures ≤about 10° C. In one embodiment, the solution or suspension is cooled to one or more temperatures in the range of ≤about 5° C. to about 10° C.

The reaction mixture may then be treated for a further period of time.

In step (c), the entrectinib anhydrate is recovered as a crystalline solid. The crystalline anhydrate may be recovered by directly by filtering, decanting or centrifuging. If desired, the suspension may be mobilised with additional portions of the solvent prior to recovery of the crystalline solid. Alternatively, a proportion or substantially all of the solvent may be evaporated prior to recovery of the crystalline solid.

Howsoever the crystalline anhydrate is recovered, the separated anhydrate may be washed with solvent (e.g. one or more of the solvents described above) and dried. Drying may be performed using known methods, for example, at temperatures in the range of about 10° C. to about 60° C., such as about 20° C. to about 40° C., for example, ambient temperature under vacuum (for example about 1 mbar to about 30 mbar) for about 1 hour to about 24 hours. Alternatively, the crystalline anhydrate may be left to dry under ambient temperature naturally i.e. without the active application of vacuum. It is preferred that the drying conditions are maintained below the point at which the anhydrate degrades and so when the anhydrate is known to degrade within the temperature or pressure ranges given above, the drying conditions should be maintained below the degradation temperature or vacuum.

Steps (a) to (c) may be carried out one or more times (e.g. 1, 2, 3, 4 or 5 times). When steps (a) to (c) are carried out more than once (e.g. 2, 3, 4 or 5 times), step (a) may be optionally seeded with crystalline entrectinib anhydrate Form C which was previously prepared and isolated by the first iteration of steps (a) to (c).

Alternatively or in addition, when steps (a) to (c) are carried out more than once (e.g. 2, 3, 4 or 5 times), the solution or suspension formed in step (b) may be optionally seeded with crystalline entrectinib anhydrate Form C (which was previously prepared and isolated by a method described herein).

The crystalline entrectinib anhydrate Form C described above may be prepared by a process comprising the steps of:
(a) contacting entrectinib in a first solvent selected from the group consisting of methyl ethyl ketone (MEK), THF, and combinations thereof;
(b) adding isopropyl acetate to the solution or suspension of entrectinib; and
(c) recovering entrectinib anhydrate Form C as a crystalline solid.

The quantity of the first solvent is not particularly limiting provided there is enough solvent to dissolve the entrectinib and form a solution, or suspend the entrectinib. The w/v ratio of entrectinib to the first solvent may be in the range of about 1 mg of entrectinib:about 1 to about 100 μl of solvent, such as about 1 mg of entrectinib:about 1 to about 50 μl of solvent, for example about 1 mg of entrectinib about 1 to about 10 μl of solvent.

The entrectinib may be dissolved in the first solvent at ambient temperature or less. In one embodiment, the contacting step may be carried out at one or more temperatures in the range of ≥about 0° C. to about ≤25° C. In some embodiments, the contacting step is carried out at one or more temperatures ≥about 1° C. In some embodiments, the contacting step is carried out at one or more temperatures ≥about 2° C. In some embodiments, the contacting step is carried out at one or more temperatures ≥about 3° C. In some embodiments, the contacting step is carried out at one or more temperatures ≥about 4° C. In some embodiments, the contacting step is carried out at one or more temperatures ≥about 5° C. In some embodiments, the contacting step is carried out at one or more temperatures ≤about 20° C. In some embodiments, the contacting step is carried out at one or more temperatures ≤about 15° C. In some embodiments, the contacting step is carried out at one or more temperatures ≤about 10° C. In one embodiment, the contacting step is carried out at one or more temperatures in the range of ≥about 0° C. to ≤about 10° C., for example, about 5° C. In one embodiment, the contacting step may be carried out at about ambient temperature e.g. about 25° C.

Alternatively, the entrectinib may be contacted in the solvent at a temperature greater than ambient i.e. greater than 30° C. and below the boiling point of the reaction mixture. The boiling point of the reaction mixture may vary depending on the pressure under which the contacting step is conducted. In one embodiment, the contacting step is carried out at atmospheric pressure (i.e. 1.0135×10⁵ Pa). In one embodiment, the contacting step may be carried out at one or more temperatures in the range of ≥about 40° C. to about ≤60° C. In some embodiments, the contacting step is carried out at one or more temperatures ≥about 41° C. In some embodiments, the contacting step is carried out at one or more temperatures ≥about 42° C. In some embodiments, the contacting step is carried out at one or more temperatures ≥about 43° C. In some embodiments, the contacting step is carried out at one or more temperatures ≥about 44° C. In some embodiments, the contacting step is carried out at one or more temperatures ≥about 45° C. In some embodiments, the contacting step is carried out at one or more temperatures ≥about 46° C. In some embodiments, the contacting step is carried out at one or more temperatures ≥about 47° C. In some embodiments, the contacting step is carried out at one or more temperatures ≥about 48° C. In some embodiments, the contacting step is carried out at one or more temperatures ≥about 49° C. In some embodiments, the contacting step is carried out at one or more temperatures ≥about 50° C. In some embodiments, the contacting step is carried out at one or more temperatures ≤about 59° C. In some embodiments, the contacting step is carried out at one or more temperatures ≤about 58° C. In some embodiments, the contacting step is carried out at one or more temperatures ≤about 57° C. In some embodiments, the contacting step is carried out at one or more temperatures ≤about 56° C. In some embodiments, the contacting step is carried out at one or more temperatures ≤about 55° C. In some embodiments, the contacting step is carried out at one or more temperatures ≤about 54° C. In some embodiments, the contacting step is carried out at one or more temperatures ≤about 53° C. In some embodiments, the contacting step is carried out at one or more temperatures ≤about 52° C. In some embodiments, the contacting step is carried out at one or more temperatures ≤about 51° C. In one embodiment, the contacting step is carried out at one or more temperatures in the range of ≥about 45° C. to ≤about 55° C. In one embodiment, the contacting step is carried out at a temperature of about 50° C.

The dissolution or suspension of entrectinib may be encouraged through the use of an aid such as stirring, shaking and/or sonication. Additional solvent may be added to aid the dissolution or suspension of the entrectinib.

The period of time for which the mixture of entrectinib and solvent is treated at the desired temperature is not particularly limiting. In one embodiment, the period of time may be from about 1 minute to about 24 minutes, for example, about 5 minutes.

In step (b), isopropyl acetate is added to the reaction mixture. The quantity of isopropyl acetate is not particularly limiting. The w/v ratio of entrectinib to isopropyl acetate may be in the range of about 1 mg of entrectinib:about 1 to about 1000 μl of isopropyl acetate, such as about 1 mg of entrectinib:about 1 to about 500 μl of isopropyl acetate, for example about 1 mg of entrectinib:about 1 to about 100 μl of isopropyl acetate, e.g. about 1 mg of entrectinib:about 25.9 μl of isopropyl acetate. These w/v ratios have been calculated using the mass of entrectinib initially dissolved in the first solvent i.e. the quantity of entrectinib inputted into the process.

After the addition of the isopropyl acetate, the reaction mixture may be treated for a period of time at ambient temperature or less as described above in connection with first solvent.

Alternatively, the entrectinib may be treated for a period of time with the isopropyl acetate at a temperature greater than ambient i.e. greater than 30° C. and below the boiling point of the reaction mixture as described above in connection with the first solvent.

The reaction mixture may then be stirred, shaken and/or sonicated for a further period of time.

The solution or suspension may then be cooled such that the resulting solution or suspension has a temperature below that of the solution or suspension of step (b). The rate of cooling may be from about 0.05° C./minute to about 2° C./minute, such as about 0.1° C./minute to about 1.5° C./minute, for example about 0.1° C./minute. When a solution of entrectinib is cooled, a suspension may eventually be observed.

The solution may be cooled to ambient temperature or a temperature of less than ambient temperature. In one embodiment, the solution or suspension may be cooled to one or more temperatures in the range of ≥about 0° C. to about ≤20° C. In some embodiments, the solution is cooled to one or more temperatures ≥about 1° C. In some embodiments, the solution is cooled to one or more temperatures ≥about 2° C. In some embodiments, the solution is cooled to one or more temperatures ≥about 3° C. In some embodiments, the solution is cooled to one or more temperatures ≥about 4° C. In some embodiments, the solution is cooled to one or more temperatures ≤about 5° C. In some embodiments, the solution is cooled to one or more temperatures ≤about 15° C. In some embodiments, the solution is cooled to one or more temperatures ≤about 14° C. In some embodiments, the solution is cooled to one or more temperatures ≤about 13° C. In some embodiments, the solution is cooled to one or more temperatures ≤about 12° C. In some embodiments, the solution may be cooled to one or more temperatures ≤about 11° C. In some embodiments, the solution is cooled to one or more temperatures ≤about 10° C. In one embodiment, the solution is cooled to one or more temperatures in the range of about 5° C. to about 10° C.

In step (c), the entrectinib anhydrate Form C is recovered as a crystalline solid. The crystalline anhydrate may be recovered by directly by filtering, decanting or centrifuging. If desired, the suspension may be mobilised with additional portions of the solvent prior to recovery of the crystalline solid. Alternatively, a proportion or substantially all of the solvent may be evaporated prior to recovery of the crystalline solid.

Howsoever the crystalline anhydrate is recovered, the separated anhydrate may be washed with solvent (e.g. one or more of the solvents described above) and dried. Drying may be performed using known methods, for example, at temperatures in the range of about 10° C. to about 60° C., such as about 20° C. to about 40° C., for example, ambient temperature under vacuum (for example about 1 mbar to about 30 mbar) for about 1 hour to about 24 hours. Alternatively, the crystalline anhydrate may be left to dry under ambient temperature naturally i.e. without the active application of vacuum. It is preferred that the drying conditions are maintained below the point at which the anhydrate degrades and so when the anhydrate is known to degrade within the temperature or pressure ranges given above, the drying conditions should be maintained below the degradation temperature or vacuum.

Steps (a) to (c) may be carried out one or more times (e.g. 1, 2, 3, 4 or 5 times). When steps (a) to (c) are carried out more than once (e.g. 2, 3, 4 or 5 times), step (a) may be optionally seeded with crystalline entrectinib anhydrate Form C which was previously prepared and isolated by a method described herein).

Alternatively or in addition, when steps (a) to (c) are carried out more than once (e.g. 2, 3, 4 or 5 times), the solution or suspension formed in step (b) may be optionally seeded with crystalline entrectinib anhydrate Form C (which was previously prepared and isolated by a method described herein).

The crystalline entrectinib anhydrate Form C described above may be prepared by a process comprising the step of exposing crystalline entrectinib isopropyl acetate solvate to water vapour.

The entrectinib solvate may be placed within an enclosed chamber under vacuum and water vapour (for instance, in the form of moist air or moist nitrogen with a relative humidity (RH) of e.g. about 75% or about 97%) may be bled into the enclosed chamber at a temperature from about ambient temperature to about 50° C., for example, about 25° C., or about 40° C. Alternatively, the entrectinib solvate may be exposed to a moist atmosphere at approximately atmospheric pressure within an enclosed chamber containing a source of liquid water at a temperature from about ambient temperature to about 50° C., for example, about 25° C., or about 40° C.

Crystalline entrectinib anhydrate Form C is produced by the described process even though water is present. Water is not incorporated into the entrectinib crystals being formed.

The period of time for which the crystalline entrectinib solvate is exposing to water vapour at the desired temperature is not particularly limiting. In one embodiment, the period of time may be from about 1 minute to about 10 days, for example, about 7 days.

In another aspect, crystalline entrectinib anhydrate Form C may be prepared by a process comprising the step of treating crystalline entrectinib isopropyl acetate solvate under vacuum.

The entrectinib solvate may be placed within an enclosed chamber under vacuum at a temperature from about ambient temperature to about 50° C., for example, about 25° C., or about 40° C.

The period of time for which the crystalline entrectinib solvate is treated under vacuum at the desired temperature is not particularly limiting. In one embodiment, the period of time may be from about 1 minute to about 7 days, for example, about 3 days.

The crystalline entrectinib anhydrate Form C formed by the processes described above may be free or substantially free of other polymorphic forms of entrectinib. In certain embodiments, the polymorphic purity of the anhydrate is ≥90%, ≥91%, ≥92%, ≥93%, ≥94%, ≥95% or higher. In certain embodiments, the polymorphic purity of the anhydrate is ≥95%. In certain embodiments, the polymorphic purity of the anhydrate is ≥96%. In certain embodiments, the polymorphic purity of the anhydrate is ≥97%. In certain embodiments, the polymorphic purity of the anhydrate is ≥98%. In certain embodiments, the polymorphic purity of the anhydrate is ≥99%.

In another aspect, the present invention relates to a pharmaceutical composition comprising crystalline entrectinib anhydrate Form C as described herein and a pharmaceutically acceptable excipient.

In another aspect, the present invention relates to a method for treating cancer in a patient comprising administering a therapeutically effective amount of crystalline entrectinib anhydrate Form C as described herein to the patient. The method of treatment includes the treatment of neuroblastoma.

In another aspect, the present invention relates to crystalline entrectinib anhydrate Form C as described herein for use in treating cancer, such as the treatment of neuroblastoma.

### Entrectinib THF Solvate

It has been discovered that entrectinib can be prepared in a well-defined and consistently reproducible tetrahydrofuran (THF) solvate form. Moreover, a reliable and scalable method for producing this solvate form has been developed. The entrectinib polymorph provided by the present invention is useful as an active ingredient in pharmaceutical formulations. In certain embodiments, the crystalline solvate form is purifiable. In certain embodiments and depending on time, temperature and humidity, the crystalline solvate form is stable. In certain embodiments, the crystalline solvate form is easy to isolate and handle. In certain embodiments, the process for preparing the crystalline solvate form is scalable.

The crystalline form described herein may be characterised using a number of methods known to the skilled person in the art, including single crystal X-ray diffraction, X-ray powder diffraction (XRPD), differential scanning calorimetry (DSC), thermal gravimetric analysis (TGA), infrared spectroscopy, Raman spectroscopy, nuclear magnetic resonance (NMR) spectroscopy (including solution and solid-state NMR). The chemical purity may be determined by standard analytical methods, such as thin layer chromatography (TLC), gas chromatography, high performance liquid chromatography (HPLC), and mass spectrometry (MS).

In one aspect, the present invention provides a crystalline form of entrectinib which is crystalline entrectinib THF solvate (designated herein as Form B).

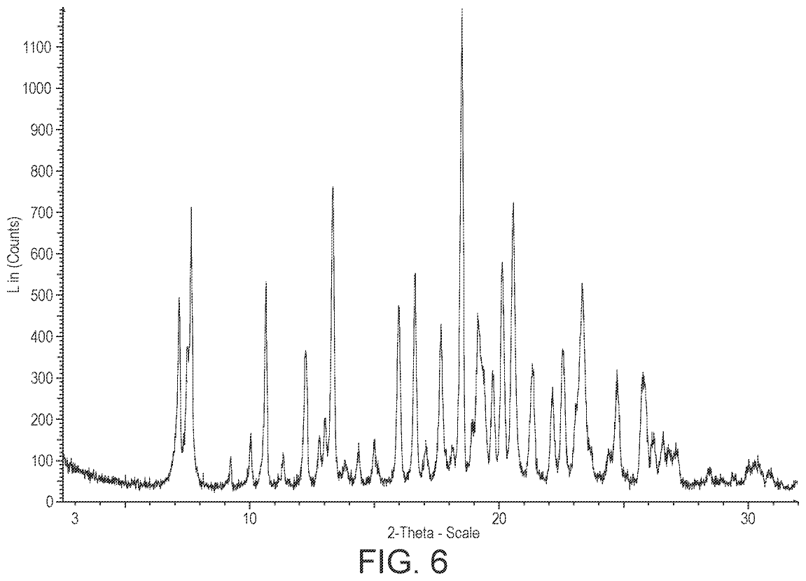

The THF solvate may have an X-ray powder diffraction pattern comprising one or more peaks (for example 1, 2, 3, 4, 5, 6, 7, 8, 9, or 10 peaks) selected from the group consisting of about 7.1, 7.6, 9.2, 10.0, 10.6, 11.3, 12.2, 12.8, 13.0, 13.3, 13.8, 14.4, 15.0, 16.0, 16.6, 17.1, 17.7, 18.1, 18.5, 18.9, 19.2, 19.4, 19.8, 20.1, 20.6, 21.3, 22.1, 22.6, 23.1, 23.4, 24.4, 24.8, 25.8, 26.2, 26.6, 27.1, 28.5, 29.4, 30.0, 30.1, 30.4, and 30.9 degrees two-theta ±0.2 degrees two-theta. In one embodiment, the solvate may have the X-ray powder diffraction pattern substantially as shown in FIG. 6.

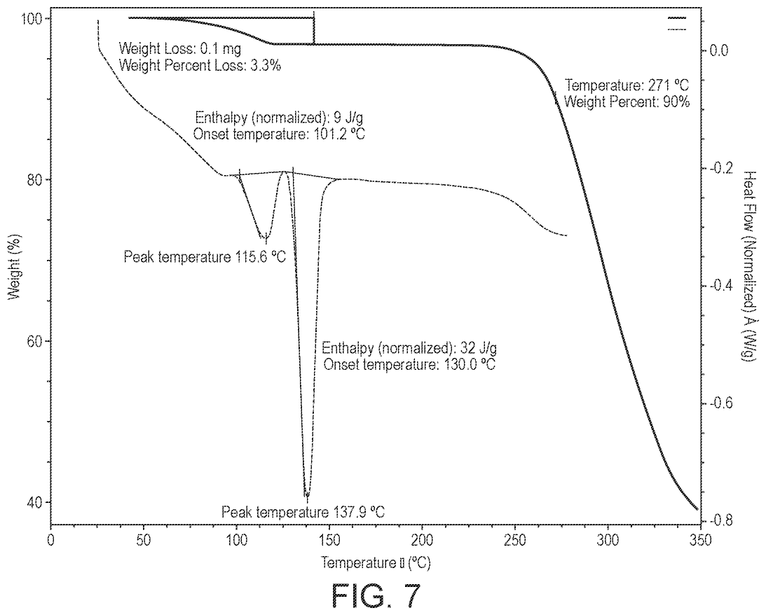

The THF solvate may have a DSC thermogram comprising two endothermic events with onset temperatures of about 101.2° C. and about 130.0° C. In one embodiment, the solvate may have a DSC thermogram substantially as shown in FIG. 7.

The THF solvate may have a TGA thermogram comprising about 3.3% mass loss when heated from about ambient temperature to about 200° C. In one embodiment, the solvate may have a TGA thermogram substantially as shown in FIG. 7.

The crystalline entrectinib THF solvate formed may be free or substantially free of other polymorphic forms of entrectinib. In certain embodiments, the polymorphic purity of the solvate is ≥90%, ≥91%, ≥92%, ≥93%, ≥94%, ≥95% or higher. In certain embodiments, the polymorphic purity of the solvate is ≥95%. In certain embodiments, the polymorphic purity of the solvate is ≥96%. In certain embodiments, the polymorphic purity of the solvate is ≥97%. In certain embodiments, the polymorphic purity of the solvate is ≥98%. In certain embodiments, the polymorphic purity of the solvate is ≥99%.

The crystalline entrectinib THF solvate Form B described above may be prepared by a process comprising the steps of:
(a) contacting entrectinib with THF; and
(b) forming a suspension of entrectinib in THF.

The quantity of THF is not particularly limiting provided there is enough THF to substantially dissolve the entrectinib. The w/v ratio of entrectinib to THF solvent may be in the range of about 1 mg of entrectinib:about 1 to about 100 μl of THF, such as about 1 mg of entrectinib:about 1 to about 50 μl of THF, for example about 1 mg of entrectinib:about 1 to about 25 μl of THF, e.g. about 1 mg of entrectinib:about 1 to about 10 μl of THF.

The entrectinib may be contacted with THF at ambient temperature or less. In one embodiment, the contacting step may be carried out at one or more temperatures in the range of ≥about 0° C. to about ≤25° C. In some embodiments, the contacting step is carried out at one or more temperatures ≥about 1° C. In some embodiments, the contacting step is carried out at one or more temperatures ≥about 2° C. In some embodiments, the contacting step is carried out at one or more temperatures ≥about 3° C. In some embodiments, the contacting step is carried out at one or more temperatures ≥about 4° C. In some embodiments, the contacting step is carried out at one or more temperatures ≥about 5° C. In some embodiments, the contacting step is carried out at one or more temperatures ≤about 20° C. In some embodiments, the contacting step is carried out at one or more temperatures ≤about 15° C. In some embodiments, the contacting step is carried out at one or more temperatures ≤about 10° C. In one embodiment, the contacting step is carried out at one or more temperatures in the range of ≥about 0° C. to ≤about 10° C., for example, about 5° C. In one embodiment, the contacting step may be carried out at ambient temperature e.g. about 25° C.

Alternatively, the entrectinib may be contacted with THF at a temperature greater than ambient i.e. greater than 30° C. and below the boiling point of the reaction mixture. The boiling point of the reaction mixture may vary depending on the pressure under which the contacting step is conducted. THF has a boiling point of about 65-67° C. at atmospheric pressure (i.e. 1.0135×10⁵ Pa). In one embodiment, the contacting step may be carried out at one or more temperatures in the range of about 30° C. to about ≤65° C. In some embodiments, the contacting step is carried out at one or more temperatures ≥about 41° C. In some embodiments, the contacting step is carried out at one or more temperatures ≥about 42° C. In some embodiments, the contacting step is carried out at one or more temperatures ≥about 43° C. In some embodiments, the contacting step is carried out at one or more temperatures ≥about 44° C. In some embodiments, the contacting step is carried out at one or more temperatures ≥about 45° C. In some embodiments, the contacting step is carried out at one or more temperatures ≥about 46° C. In some embodiments, the contacting step is carried out at one or more temperatures ≥about 47° C. In some embodiments, the contacting step is carried out at one or more temperatures ≥about 48° C. In some embodiments, the contacting step is carried out at one or more temperatures ≥about 49° C. In some embodiments, the contacting step is carried out at one or more temperatures ≥about 50° C. In some embodiments, the contacting step is carried out at one or more temperatures ≤about 60° C. In some embodiments, the contacting step is carried out at one or more temperatures ≤about 59° C. In some embodiments, the contacting step is carried out at one or more temperatures ≤about 58° C. In some embodiments, the contacting step is carried out at one or more temperatures ≤about 57° C. In some embodiments, the contacting step is carried out at one or more temperatures ≤about 56° C. In some embodiments, the contacting step is carried out at one or more temperatures ≤about 55° C. In some embodiments, the contacting step is carried out at one or more temperatures ≤about 54° C. In some embodiments, the contacting step is carried out at one or more temperatures ≤about 53° C. In some embodiments, the contacting step is carried out at one or more temperatures ≤about 52° C. In some embodiments, the contacting step is carried out at one or more temperatures ≤about 51° C. In one embodiment, the contacting step is carried out at one or more temperatures in the range of ≥about 45° C. to ≤about 55° C. In one embodiment, the contacting step is carried out at a temperature of about 50° C.

The solution or suspension of entrectinib may be agitated through the use of an aid such as stirring, shaking and/or sonication.

The period of time for which the mixture of entrectinib and solvent is treated at the desired temperature is not particularly limiting. In one embodiment, the period of time may be from about 1 minute to about 24 minutes, for example, about 1 hour.

The process may further comprise the step of adding water to the reaction mixture, optionally when the reaction mixture is at a temperature greater than ambient temperature as described above. The quantity of water is not particularly limiting. The w/v ratio of entrectinib to water may be in the range of about 1 mg of entrectinib:about 1 to about 1000 μl of water, such as about 1 mg of entrectinib:about 1 to about 100 μl of water, for example about 1 mg of entrectinib:about 1 to about 15 μl of water, e.g. about 1 mg of entrectinib:about 10 μl of water. These w/v ratios have been calculated using the mass of entrectinib initially dissolved in THF i.e. the quantity of entrectinib inputted into the process.

The reaction mixture may then be treated for a further period of time e.g. about 1 minute to about 24 hours, such as about 1 hour.

The solution or suspension may then be cooled such that the resulting solution or suspension has a temperature below that of the solution or suspension of step (b). The rate of cooling may be from about 0.05° C./minute to about 2° C./minute, such as about 0.1° C./minute to about 1.5° C./minute, for example about 0.1° C./minute. When a solution of entrectinib is cooled, a suspension may eventually be observed.

The solution or suspension may be cooled to ambient temperature or a temperature of less than ambient temperature. In one embodiment, the solution or suspension may be cooled to one or more temperatures in the range of ≥about 0° C. to about ≤20° C. In some embodiments, the solution is cooled to one or more temperatures ≥about 1° C. In some embodiments, the solution is cooled to one or more temperatures ≥about 2° C. In some embodiments, the solution is cooled to one or more temperatures ≥about 3° C. In some embodiments, the solution is cooled to one or more temperatures ≥about 4° C. In some embodiments, the solution is cooled to one or more temperatures ≥about 5° C. In some embodiments, the solution is cooled to one or more temperatures ≤about 15° C. In some embodiments, the solution is cooled to one or more temperatures ≤about 14° C. In some embodiments, the solution is cooled to one or more temperatures ≤about 13° C. In some embodiments, the solution is cooled to one or more temperatures ≤about 12° C. In some embodiments, the solution may be cooled to one or more temperatures ≤about 11° C. In some embodiments, the solution is cooled to one or more temperatures ≤about 10° C. In one embodiment, the solution is cooled to one or more temperatures in the range of about 5° C. to about 10° C.

The process may further comprise the step of recovering entrectinib THF solvate as a crystalline solid. The crystalline solvate may be recovered by directly by filtering, decanting or centrifuging. If desired, the suspension may be mobilised with additional portions of THF and/or water prior to recovery of the crystalline solid. Alternatively, a proportion or substantially all of the THF solvent or THF/water solvent mixture may be evaporated prior to recovery of the crystalline solid.

Howsoever the crystalline THF solvate is recovered, the separated solvate may be washed with solvent (e.g. THF and/or water) and dried. Drying may be performed using known methods, for example, at temperatures in the range of about 10° C. to about 60° C., such as about 20° C. to about 40° C., for example, ambient temperature under vacuum (for example about 1 mbar to about 30 mbar) for about 1 hour to about 24 hours. It is preferred that the drying conditions are maintained below the point at which the THF solvate desolvates and so when the solvate is known to desolvate within the temperature or pressure ranges given above, the drying conditions should be maintained below the desolvation temperature or vacuum.

The crystalline entrectinib THF solvate Form B formed by the process described above may be free or substantially free of other polymorphic forms of entrectinib. In certain embodiments, the polymorphic purity of the solvate is ≥90%, ≥91%, ≥92%, ≥93%, ≥94%, ≥95% or higher. In certain embodiments, the polymorphic purity of the solvate is ≥95%. In certain embodiments, the polymorphic purity of the solvate is ≥96%. In certain embodiments, the polymorphic purity of the solvate is ≥97%. In certain embodiments, the polymorphic purity of the solvate is ≥98%. In certain embodiments, the polymorphic purity of the solvate is ≥99%.

### Entrectinib Nitromethane Solvate

It has been discovered that entrectinib can be prepared in a well-defined and consistently reproducible nitromethane solvate form. Moreover, a reliable and scalable method for producing this solvate form has been developed. The entrectinib polymorph provided by the present invention is useful as an active ingredient in pharmaceutical formulations. In certain embodiments, the crystalline solvate form is purifiable. In certain embodiments and depending on time, temperature and humidity, the crystalline solvate form is stable. In certain embodiments, the crystalline solvate form is easy to isolate and handle.

In certain embodiments, the process for preparing the crystalline solvate form is scalable. The crystalline form described herein may be characterised using a number of methods known to the skilled person in the art, including single crystal X-ray diffraction, X-ray powder diffraction (XRPD), differential scanning calorimetry (DSC), thermal gravimetric analysis (TGA), infrared spectroscopy, Raman spectroscopy, nuclear magnetic resonance (NMR) spectroscopy (including solution and solid-state NMR). The chemical purity may be determined by standard analytical methods, such as thin layer chromatography (TLC), gas chromatography, high performance liquid chromatography (HPLC), and mass spectrometry (MS).

In one aspect, the present invention provides a crystalline form of entrectinib which is crystalline entrectinib nitromethane solvate (designated herein as Form D).

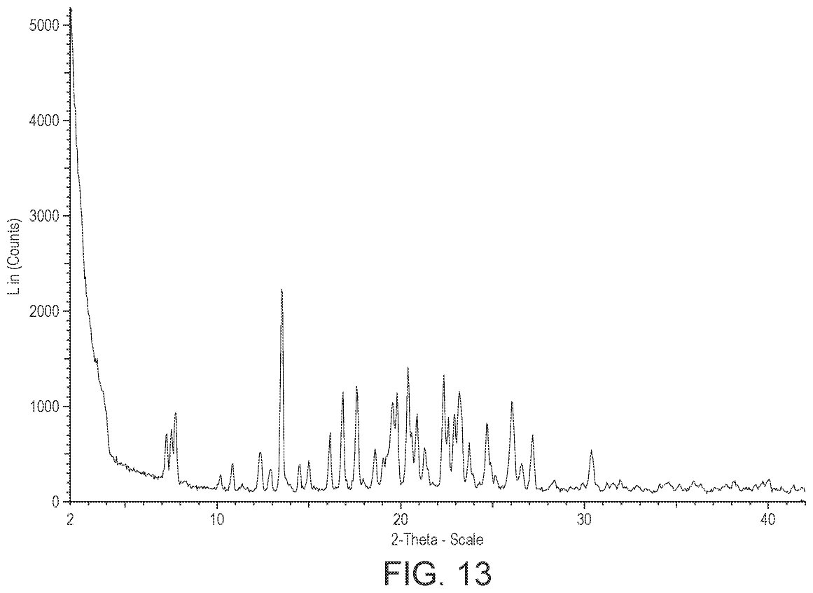

The nitromethane solvate may have an X-ray powder diffraction pattern comprising one or more peaks (for example 1, 2, 3, 4, 5, 6, 7, 8, 9, or 10 peaks) selected from the group consisting of about 7.2, 7.5, 7.7, 10.2, 10.8, 12.3, 12.9, 13.5, 14.0, 14.5, 15.0, 16.1, 16.8, 17.6, 18.0, 18.6, 19.0, 19.6, 19.8, 20.4, 20.6, 20.9, 21.3, 21.5, 22.3, 22.6, 22.9, 23.2, 23.7, 23.9, 24.7, 24.9, 25.1, 26.0, 26.6, 27.2, 28.4, and 30.4 degrees two-theta ±0.2 degrees two-theta. In one embodiment, the solvate may have the X-ray powder diffraction pattern substantially as shown in FIG. 13.

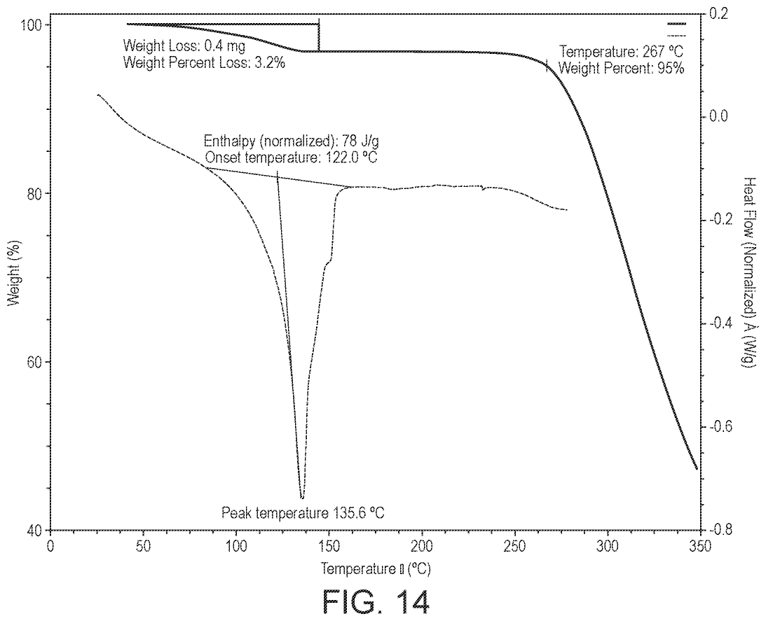

The nitromethane solvate may have a DSC thermogram comprising an endothermic event with an onset temperature of about 122.0° C. In one embodiment, the solvate may have a DSC thermogram substantially as shown in FIG. 14.

The nitromethane solvate may have a TGA thermogram comprising about 3.2% mass loss when heated from about ambient temperature to about 200° C. In one embodiment, the solvate may have a TGA thermogram substantially as shown in FIG. 14.

The crystalline entrectinib nitromethane solvate formed may be free or substantially free of other polymorphic forms of entrectinib. In certain embodiments, the polymorphic purity of the solvate is ≥90%, ≥91%, ≥92%, ≥93%, ≥94%, ≥95% or higher. In certain embodiments, the polymorphic purity of the solvate is ≥95%. In certain embodiments, the polymorphic purity of the solvate is 96≥%. In certain embodiments, the polymorphic purity of the solvate is ≥97%. In certain embodiments, the polymorphic purity of the solvate is ≥98%. In certain embodiments, the polymorphic purity of the solvate is ≥99%.

The crystalline entrectinib nitromethane solvate described above may be prepared by a process comprising the steps of:
(a) contacting entrectinib with nitromethane; and
(b) forming a suspension of entrectinib in nitromethane.

The quantity of nitromethane is not particularly limiting provided there is enough nitromethane to substantially suspend the entrectinib. The w/v ratio of entrectinib to nitromethane solvent may be in the range of about 1 mg of entrectinib:about 1 to about 1000 μl of nitromethane, such as about 1 mg of entrectinib:about 1 to about 500 μl of nitromethane, for example about 1 mg of entrectinib:about 1 to about 250 μl of nitromethane, e.g. about 1 mg of entrectinib:about 5 to about 110 μl of nitromethane.

The entrectinib may be contacted with nitromethane at ambient temperature or less. In one embodiment, the contacting step may be carried out at one or more temperatures in the range of about 0° C. to about ≤25° C. In some embodiments, the contacting step is carried out at one or more temperatures ≥about 1° C. In some embodiments, the contacting step is carried out at one or more temperatures ≥about 2° C. In some embodiments, the contacting step is carried out at one or more temperatures ≥about 3° C. In some embodiments, the contacting step is carried out at one or more temperatures ≥about 4° C. In some embodiments, the contacting step is carried out at one or more temperatures ≥about 5° C. In some embodiments, the contacting step is carried out at one or more temperatures ≤about 20° C. In some embodiments, the contacting step is carried out at one or more temperatures ≤about 15° C. In some embodiments, the contacting step is carried out at one or more temperatures ≤about 10° C. In one embodiment, the contacting step is carried out at one or more temperatures in the range of ≥about 0° C. to ≤about 10° C., for example, about 5° C. In one embodiment, the contacting step may be carried out at ambient temperature e.g. about 25° C.

Alternatively, the entrectinib may be contacted with nitromethane at a temperature greater than ambient i.e. greater than 30° C. and below the boiling point of the reaction mixture. The boiling point of the reaction mixture may vary depending on the pressure under which the contacting step is conducted. Nitromethane has a boiling point of about 101.2° C. at atmospheric pressure (i.e. 1.0135×10⁵ Pa). In one embodiment, the contacting step may be carried out at one or more temperatures in the range of ≥about 30° C. to about ≤70° C. In some embodiments, the contacting step is carried out at one or more temperatures ≥about 41° C. In some embodiments, the contacting step is carried out at one or more temperatures ≥about 42° C. In some embodiments, the contacting step is carried out at one or more temperatures ≥about 43° C. In some embodiments, the contacting step is carried out at one or more temperatures ≥about 44° C. In some embodiments, the contacting step is carried out at one or more temperatures ≥about 45° C. In some embodiments, the contacting step is carried out at one or more temperatures ≥about 46° C. In some embodiments, the contacting step is carried out at one or more temperatures ≥about 47° C. In some embodiments, the contacting step is carried out at one or more temperatures ≥about 48° C. In some embodiments, the contacting step is carried out at one or more temperatures ≥about 49° C. In some embodiments, the contacting step is carried out at one or more temperatures ≥about 50° C. In some embodiments, the contacting step is carried out at one or more temperatures ≤about 70° C. In some embodiments, the contacting step is carried out at one or more temperatures ≤about 69° C. In some embodiments, the contacting step is carried out at one or more temperatures ≤about 68° C. In some embodiments, the contacting step is carried out at one or more temperatures ≤about 67° C. In some embodiments, the contacting step is carried out at one or more temperatures ≤about 66° C. In some embodiments, the contacting step is carried out at one or more temperatures ≤about 65° C. In some embodiments, the contacting step is carried out at one or more temperatures ≤about 64° C. In some embodiments, the contacting step is carried out at one or more temperatures ≤about 63° C. In some embodiments, the contacting step is carried out at one or more temperatures ≤about 62° C. In some embodiments, the contacting step is carried out at one or more temperatures ≤about 61° C. In one embodiment, the contacting step is carried out at one or more temperatures in the range of ≥about 45° C. to ≤about 65° C. In one embodiment, the contacting step is carried out at a temperature of about 50° C. In another embodiment, the contacting step is carried out at a temperature of about 60° C.

The period of time for which the mixture of entrectinib and solvent is treated at the desired temperature is not particularly limiting. In one embodiment, the period of time may be from about 1 minute to about 7 days, for example, about 3 days.

Alternatively, entrectinib may be contacted with nitromethane at ambient temperature or less and then matured between this temperature and one or more temperatures greater than ambient temperature. Ambient temperature, temperatures less than ambient, and temperatures greater than ambient are as described above. The maturation step may comprise oscillating the temperature for a period of time (e.g. about 4 hours) at ambient, a period of time at the temperature greater than ambient (e.g. about 4 hours), followed by another period of time (e.g. about 4 hours) at ambient, and so on, for an extended period of time (e.g. about 6 days).

The suspension of entrectinib may be agitated through the use of an aid such as stirring, shaking and/or sonication.

The process may further comprise the step of recovering entrectinib nitromethane solvate as a crystalline solid. The crystalline solvate may be recovered by directly by filtering, decanting or centrifuging. If desired, the suspension may be mobilised with additional portions of nitromethane prior to recovery of the crystalline solid. Alternatively, a proportion or substantially all of the nitromethane solvent may be evaporated prior to recovery of the crystalline solid.

Howsoever the crystalline nitromethane solvate is recovered, the separated solvate may be washed with solvent (e.g. nitromethane, methyl ethyl ketone, THF, toluene, DMF, propanol (e.g. 1- or 2-propanol), or a mixture thereof) and dried. Drying may be performed using known methods, for example, at temperatures in the range of about 10° C. to about 60° C., such as about 20° C. to about 40° C., for example, ambient temperature under vacuum (for example about 1 mbar to about 30 mbar) for about 1 hour to about 24 hours. It is preferred that the drying conditions are maintained below the point at which the nitromethane solvate desolvates and so when the solvate is known to desolvate within the temperature or pressure ranges given above, the drying conditions should be maintained below the desolvation temperature or vacuum.

The crystalline entrectinib nitromethane solvate Form D described above may be prepared by a process comprising the steps of:
(a) contacting entrectinib in a first solvent selected from the group consisting of methyl ethyl ketone (MEK), THF, toluene, DMF, propanol (such as a 1-propanol or 2-propanol) and combinations thereof;
(b) adding nitromethane to the solution or suspension of entrectinib; and
(c) recovering entrectinib nitromethane solvate Form D as a crystalline solid.

The quantity of the first solvent is not particularly limiting provided there is enough solvent to dissolve the entrectinib and form a solution, or suspend the entrectinib. The w/v ratio of entrectinib to the first solvent may be in the range of about 1 mg of entrectinib:about 1 to about 1000 μl of solvent, such as about 1 mg of entrectinib:about 1 to about 500 μl of solvent, for example about 1 mg of entrectinib about 5 to about 150 μl of solvent.

The entrectinib may be dissolved in the first solvent at ambient temperature or less. In one embodiment, the contacting step may be carried out at one or more temperatures in the range of ≥about 0° C. to about ≤25° C. In some embodiments, the contacting step is carried out at one or more temperatures ≥about 1° C. In some embodiments, the contacting step is carried out at one or more temperatures ≥about 2° C. In some embodiments, the contacting step is carried out at one or more temperatures ≥about 3° C. In some embodiments, the contacting step is carried out at one or more temperatures ≥about 4° C. In some embodiments, the contacting step is carried out at one or more temperatures ≥about 5° C. In some embodiments, the contacting step is carried out at one or more temperatures ≤about 20° C. In some embodiments, the contacting step is carried out at one or more temperatures ≤about 15° C. In some embodiments, the contacting step is carried out at one or more temperatures ≤about 10° C. In one embodiment, the contacting step is carried out at one or more temperatures in the range of ≥about 0° C. to ≤about 10° C., for example, about 5° C. In one embodiment, the contacting step may be carried out at about ambient temperature e.g. about 25° C.

Alternatively, the entrectinib may be contacted in the solvent at a temperature greater than ambient i.e. greater than 30° C. and below the boiling point of the reaction mixture. The boiling point of the reaction mixture may vary depending on the pressure under which the contacting step is conducted. In one embodiment, the contacting step is carried out at atmospheric pressure (i.e. 1.0135×10⁵ Pa). In one embodiment, the contacting step may be carried out at one or more temperatures in the range of about 40° C. to about ≤60° C. In some embodiments, the contacting step is carried out at one or more temperatures ≥about 41° C. In some embodiments, the contacting step is carried out at one or more temperatures ≥about 42° C. In some embodiments, the contacting step is carried out at one or more temperatures ≥about 43° C. In some embodiments, the contacting step is carried out at one or more temperatures ≥about 44° C. In some embodiments, the contacting step is carried out at one or more temperatures ≥about 45° C. In some embodiments, the contacting step is carried out at one or more temperatures ≥about 46° C. In some embodiments, the contacting step is carried out at one or more temperatures ≥about 47° C. In some embodiments, the contacting step is carried out at one or more temperatures ≥about 48° C. In some embodiments, the contacting step is carried out at one or more temperatures ≥about 49° C. In some embodiments, the contacting step is carried out at one or more temperatures ≥about 50° C. In some embodiments, the contacting step is carried out at one or more temperatures ≤about 59° C. In some embodiments, the contacting step is carried out at one or more temperatures ≤about 58° C. In some embodiments, the contacting step is carried out at one or more temperatures ≤about 57° C. In some embodiments, the contacting step is carried out at one or more temperatures ≤about 56° C. In some embodiments, the contacting step is carried out at one or more temperatures ≤about 55° C. In some embodiments, the contacting step is carried out at one or more temperatures ≤about 54° C. In some embodiments, the contacting step is carried out at one or more temperatures ≤about 53° C. In some embodiments, the contacting step is carried out at one or more temperatures ≤about 52° C. In some embodiments, the contacting step is carried out at one or more temperatures ≤about 51° C. In one embodiment, the contacting step is carried out at one or more temperatures in the range of ≥about 45° C. to ≤about 55° C. In one embodiment, the contacting step is carried out at a temperature of about 50° C.

The dissolution or suspension of entrectinib may be encouraged through the use of an aid such as stirring, shaking and/or sonication. Additional solvent may be added to aid the dissolution or suspension of the entrectinib.

The period of time for which the mixture of entrectinib and solvent is treated at the desired temperature is not particularly limiting. In one embodiment, the period of time may be from about 1 minute to about 24 hours, for example, about 5 minutes.

In step (b), nitromethane is added to the reaction mixture. The quantity of nitromethane is not particularly limiting. The w/v ratio of entrectinib to nitromethane may be in the range of about 1 mg of entrectinib:about 1 to about 1000 μl of nitromethane, such as about 1 mg of entrectinib:about 1 to about 500 μl of nitromethane, for example about 1 mg of entrectinib:about 1 to about 100 μl of nitromethane, e.g. about 5 mg of entrectinib:about 30 μl of nitromethane. These w/v ratios have been calculated using the mass of entrectinib initially dissolved in the first solvent i.e. the quantity of entrectinib inputted into the process.

The period of time for which the mixture of entrectinib and solvent mixture is treated at the desired temperature is not particularly limiting. In one embodiment, the period of time may be from about 1 minute to about 24 hours, for example, about 1 hour.

After the addition of the nitromethane, the reaction mixture may be treated for a period of time at ambient temperature or less as described above in connection with first solvent.

Alternatively, the reaction mixture may be treated for a period of time at a temperature greater than ambient i.e. greater than 30° C. and below the boiling point of the reaction mixture as described above in connection with the first solvent.

The reaction mixture may be left for a further period of time, e.g. about 1 minute to about 24 hours, such as about 1 hour.

The solution or suspension may then be cooled such that the resulting solution or suspension has a temperature below that of the solution or suspension of step (b). The rate of cooling may be from about 0.05° C./minute to about 2° C./minute, such as about 0.1° C./minute to about 1.5° C./minute, for example about 0.1° C./minute. When a solution of entrectinib is cooled, a suspension may eventually be observed.

The solution or suspension may be cooled to ambient temperature or a temperature of less than ambient temperature. In one embodiment, the solution or suspension may be cooled to one or more temperatures in the range of ≥about 0° C. to about ≤20° C. In some embodiments, the solution or suspension is cooled to one or more temperatures ≥about 1° C. In some embodiments, the solution or suspension is cooled to one or more temperatures ≥about 2° C. In some embodiments, the solution or suspension is cooled to one or more temperatures ≥about 3° C. In some embodiments, the solution or suspension is cooled to one or more temperatures ≥about 4° C. In some embodiments, the solution or suspension is cooled to one or more temperatures ≥about 5° C. In some embodiments, the solution or suspension is cooled to one or more temperatures ≤about 15° C. In some embodiments, the solution or suspension is cooled to one or more temperatures ≤about 14° C. In some embodiments, the solution or suspension is cooled to one or more temperatures ≤about 13° C. In some embodiments, the solution or suspension is cooled to one or more temperatures ≤about 12° C. In some embodiments, the solution or suspension may be cooled to one or more temperatures ≤about 11° C. In some embodiments, the solution or suspension is cooled to one or more temperatures ≤about 10° C. In one embodiment, the solution or suspension is cooled to one or more temperatures in the range of about 5° C. to about 10° C.

In step (c), the entrectinib nitromethane solvate Form D is recovered as a crystalline solid. The crystalline solvate may be recovered by directly by filtering, decanting or centrifuging. If desired, the suspension may be mobilised with additional portions of the solvent prior to recovery of the crystalline solid. Alternatively, a proportion or substantially all of the solvent may be evaporated prior to recovery of the crystalline solid.

Howsoever the crystalline solvate is recovered, the separated solvate may be washed with solvent (e.g. one or more of the solvents described above) and dried. Drying may be performed using known methods, for example, at temperatures in the range of about 10° C. to about 60° C., such as about 20° C. to about 40° C., for example, ambient temperature under vacuum (for example about 1 mbar to about 30 mbar) for about 1 hour to about 24 hours. Alternatively, the crystalline solvate may be left to dry under ambient temperature naturally i.e. without the active application of vacuum. It is preferred that the drying conditions are maintained below the point at which the solvate degrades and so when the solvate is known to degrade within the temperature or pressure ranges given above, the drying conditions should be maintained below the degradation temperature or vacuum.

Steps (a) to (c) may be carried out one or more times (e.g. 1, 2, 3, 4 or 5 times). When steps (a) to (c) are carried out more than once (e.g. 2, 3, 4 or 5 times), step (a) may be optionally seeded with crystalline entrectinib nitromethane solvate Form D which was previously prepared and isolated by a method described herein).

Alternatively or in addition, when steps (a) to (c) are carried out more than once (e.g. 2, 3, 4 or 5 times), the solution or suspension formed in step (b) may be optionally seeded with crystalline entrectinib nitromethane solvate Form D (which was previously prepared and isolated by a method described herein).

The crystalline entrectinib nitromethane solvate Form D formed by the processes described above may be free or substantially free of other polymorphic forms of entrectinib. In certain embodiments, the polymorphic purity of the solvate is ≥90%, ≥91%, ≥92%, ≥93%, ≥94%, ≥95% or higher. In certain embodiments, the polymorphic purity of the solvate is ≥95%. In certain embodiments, the polymorphic purity of the solvate is ≥96%. In certain embodiments, the polymorphic purity of the solvate is ≥97%. In certain embodiments, the polymorphic purity of the solvate is ≥98%. In certain embodiments, the polymorphic purity of the solvate is ≥99%.

### Entrectinib Acetonitrile Solvate

It has been discovered that entrectinib can be prepared in a well-defined and consistently reproducible acetonitrile solvate form. Moreover, a reliable and scalable method for producing this solvate form has been developed. The entrectinib polymorph provided by the present invention is useful as an active ingredient in pharmaceutical formulations. In certain embodiments, the crystalline solvate form is purifiable. In certain embodiments and depending on time, temperature and humidity, the crystalline solvate form is stable. In certain embodiments, the crystalline solvate form is easy to isolate and handle.

In certain embodiments, the process for preparing the crystalline solvate form is scalable. The crystalline form described herein may be characterised using a number of methods known to the skilled person in the art, including single crystal X-ray diffraction, X-ray powder diffraction (XRPD), differential scanning calorimetry (DSC), thermal gravimetric analysis (TGA), infrared spectroscopy, Raman spectroscopy, nuclear magnetic resonance (NMR) spectroscopy (including solution and solid-state NMR). The chemical purity may be determined by standard analytical methods, such as thin layer chromatography (TLC), gas chromatography, high performance liquid chromatography (HPLC), and mass spectrometry (MS).

In one aspect, the present invention provides a crystalline form of entrectinib which is crystalline entrectinib acetonitrile solvate (designated herein as Form E).

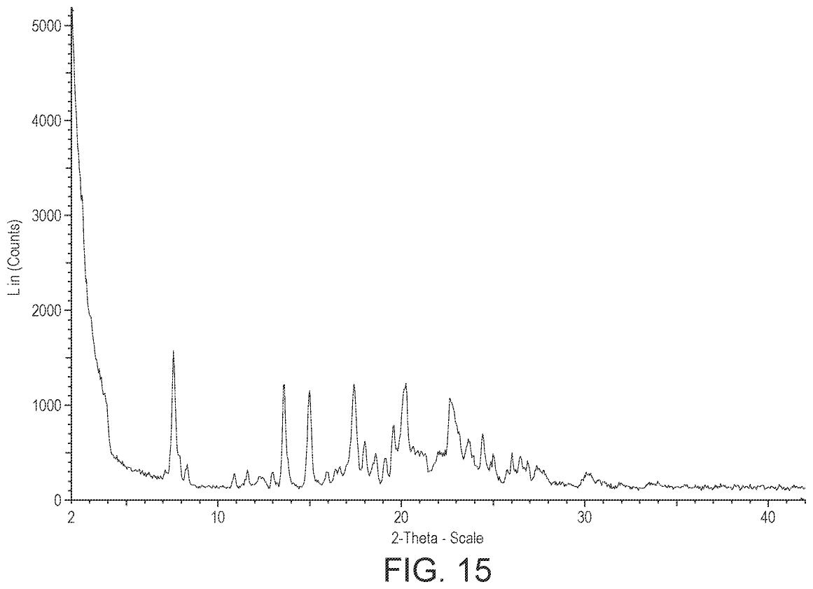

The acetonitrile solvate may have an X-ray powder diffraction pattern comprising one or more peaks (for example 1, 2, 3, 4, 5, 6, 7, 8, 9, or 10 peaks) selected from the group consisting of about 7.1, 7.5, 7.8, 8.3, 10.8, 11.6, 12.2, 12.4, 13.0, 13.6, 15.0, 15.9, 16.4, 16.6, 17.0, 17.4, 18.0, 18.4, 18.6, 19.1, 19.6, 20.2, 20.6, 20.9, 21.1, 21.3, 21.8, 22.0, 22.7, 23.7, 24.4, 25.0, 25.7, 26.0, 26.5, 26.9, 27.4, and 27.8 degrees two-theta ±0.2 degrees two-theta. In one embodiment, the solvate may have the X-ray powder diffraction pattern substantially as shown in FIG. 15.

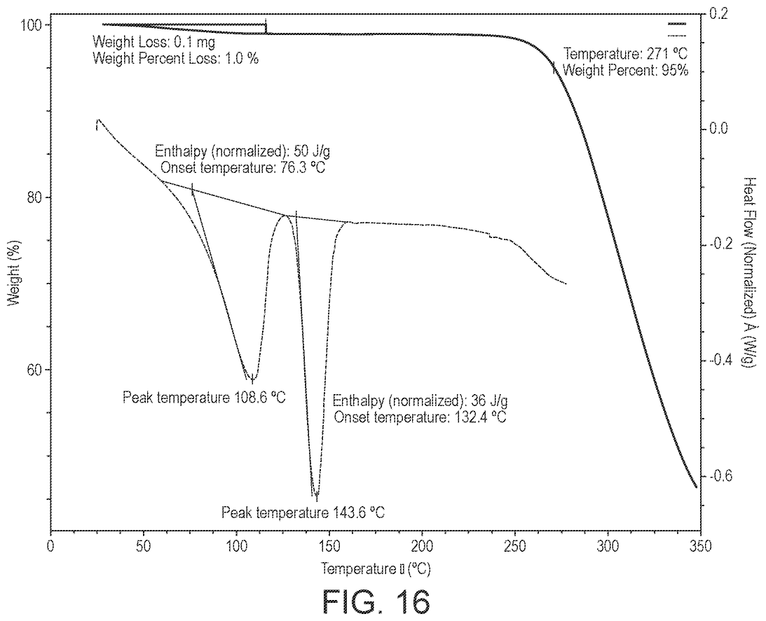

The acetonitrile solvate may have a DSC thermogram comprising two endothermic events with onset temperatures of about 76.3° C. and about 132.4° C. In one embodiment, the solvate may have a DSC thermogram substantially as shown in FIG. 16.

The acetonitrile solvate may have a TGA thermogram comprising about 1.0% mass loss when heated from about ambient temperature to about 200° C. In one embodiment, the solvate may have a TGA thermogram substantially as shown in FIG. 16.

The crystalline entrectinib acetonitrile solvate formed may be free or substantially free of other polymorphic forms of entrectinib. In certain embodiments, the polymorphic purity of the solvate is ≥90%, ≥91%, ≥92%, ≥93%, ≥94%, ≥95% or higher. In certain embodiments, the polymorphic purity of the solvate is ≥95%. In certain embodiments, the polymorphic purity of the solvate is 96≥%. In certain embodiments, the polymorphic purity of the solvate is ≥97%. In certain embodiments, the polymorphic purity of the solvate is ≥98%. In certain embodiments, the polymorphic purity of the solvate is ≥99%.

The crystalline entrectinib acetonitrile solvate Form E described above may be prepared by a process comprising the steps of:
(a) contacting entrectinib with acetonitrile; and
(b) forming a suspension of entrectinib in acetonitrile.

The quantity of acetonitrile is not particularly limiting provided there is enough acetonitrile to substantially suspend the entrectinib. The w/v ratio of entrectinib to acetonitrile solvent may be in the range of about 1 mg of entrectinib:about 1 to about 1000 μl of acetonitrile, such as about 1 mg of entrectinib:about 1 to about 500 μl of acetonitrile, for example about 1 mg of entrectinib:about 1 to about 250 μl of acetonitrile, e.g. about 1 mg of entrectinib:about 5 to about 100 μl of acetonitrile.

The entrectinib may be contacted with acetonitrile at ambient temperature or less. In one embodiment, the contacting step may be carried out at one or more temperatures in the range of ≥about 0° C. to about ≤25° C. In some embodiments, the contacting step is carried out at one or more temperatures ≥about 1° C. In some embodiments, the contacting step is carried out at one or more temperatures ≥about 2° C. In some embodiments, the contacting step is carried out at one or more temperatures ≥about 3° C. In some embodiments, the contacting step is carried out at one or more temperatures ≥about 4° C. In some embodiments, the contacting step is carried out at one or more temperatures ≥about 5° C. In some embodiments, the contacting step is carried out at one or more temperatures ≤about 20° C. In some embodiments, the contacting step is carried out at one or more temperatures ≤about 15° C. In some embodiments, the contacting step is carried out at one or more temperatures ≤about 10° C. In one embodiment, the contacting step is carried out at one or more temperatures in the range of ≥about 0° C. to ≤about 10° C., for example, about 5° C. In one embodiment, the contacting step may be carried out at ambient temperature e.g. about 25° C.

Alternatively, the entrectinib may be contacted with acetonitrile at a temperature greater than ambient i.e. greater than 30° C. and below the boiling point of the reaction mixture. The boiling point of the reaction mixture may vary depending on the pressure under which the contacting step is conducted. Acetonitrile has a boiling point of about 81-82° C. at atmospheric pressure (i.e. 1.0135×10⁵ Pa). In one embodiment, the contacting step may be carried out at one or more temperatures in the range of about 30° C. to about ≤70° C. In some embodiments, the contacting step is carried out at one or more temperatures ≥about 41° C. In some embodiments, the contacting step is carried out at one or more temperatures ≥about 42° C. In some embodiments, the contacting step is carried out at one or more temperatures ≥about 43° C. In some embodiments, the contacting step is carried out at one or more temperatures ≥about 44° C. In some embodiments, the contacting step is carried out at one or more temperatures ≥about 45° C. In some embodiments, the contacting step is carried out at one or more temperatures ≥about 46° C. In some embodiments, the contacting step is carried out at one or more temperatures ≥about 47° C. In some embodiments, the contacting step is carried out at one or more temperatures ≥about 48° C. In some embodiments, the contacting step is carried out at one or more temperatures ≥about 49° C. In some embodiments, the contacting step is carried out at one or more temperatures ≥about 50° C. In some embodiments, the contacting step is carried out at one or more temperatures ≤about 70° C. In some embodiments, the contacting step is carried out at one or more temperatures ≤about 69° C. In some embodiments, the contacting step is carried out at one or more temperatures ≤about 68° C. In some embodiments, the contacting step is carried out at one or more temperatures ≤about 67° C. In some embodiments, the contacting step is carried out at one or more temperatures ≤about 66° C. In some embodiments, the contacting step is carried out at one or more temperatures ≤about 65° C. In some embodiments, the contacting step is carried out at one or more temperatures ≤about 64° C. In some embodiments, the contacting step is carried out at one or more temperatures ≤about 63° C. In some embodiments, the contacting step is carried out at one or more temperatures ≤about 62° C. In some embodiments, the contacting step is carried out at one or more temperatures ≤about 61° C. In one embodiment, the contacting step is carried out at one or more temperatures in the range of ≥about 45° C. to ≤about 65° C. In one embodiment, the contacting step is carried out at a temperature of about 50° C. In another embodiment, the contacting step is carried out at a temperature of about 60° C.

The period of time for which the mixture of entrectinib and solvent is treated at the desired temperature is not particularly limiting. In one embodiment, the period of time may be from about 1 minute to about 7 days, for example, about 3 days.

Alternatively, entrectinib may be contacted with acetonitrile at ambient temperature or less and then matured between this temperature and one or more temperatures greater than ambient temperature. Ambient temperature, temperatures less than ambient, and temperatures greater than ambient are as described above. The maturation step may comprise oscillating the temperature for a period of time (e.g. about 4 hours) at ambient, a period of time at the temperature greater than ambient (e.g. about 4 hours), followed by another period of time (e.g. about 4 hours) at ambient, and so on, for an extended period of time (e.g. about 6 days).

The suspension of entrectinib may be agitated through the use of an aid such as stirring, shaking and/or sonication.

The process may further comprise the step of recovering entrectinib acetonitrile solvate as a crystalline solid. The crystalline solvate may be recovered by directly by filtering, decanting or centrifuging. If desired, the suspension may be mobilised with additional portions of acetonitrile prior to recovery of the crystalline solid. Alternatively, a proportion or substantially all of the acetonitrile solvent may be evaporated prior to recovery of the crystalline solid.

Howsoever the crystalline acetonitrile solvate is recovered, the separated solvate may be washed with solvent (e.g. acetonitrile) and dried. Drying may be performed using known methods, for example, at temperatures in the range of about 10° C. to about 60° C., such as about 20° C. to about 40° C., for example, ambient temperature under vacuum (for example about 1 mbar to about 30 mbar) for about 1 hour to about 24 hours. It is preferred that the drying conditions are maintained below the point at which the acetonitrile solvate desolvates and so when the solvate is known to desolvate within the temperature or pressure ranges given above, the drying conditions should be maintained below the desolvation temperature or vacuum.

The crystalline entrectinib acetonitrile solvate Form E described above may be prepared by a process comprising the steps of:
(a) contacting entrectinib in a first solvent selected from the group consisting of methyl ethyl ketone (MEK), THF, DMF, propanol (such as a 1-propanol or 2-propanol), and combinations thereof;
(b) adding acetonitrile to the solution or suspension of entrectinib; and
(c) recovering entrectinib acetonitrile solvate Form E as a crystalline solid.

The quantity of the first solvent is not particularly limiting provided there is enough solvent to dissolve the entrectinib and form a solution, or suspend the entrectinib. The w/v ratio of entrectinib to the first solvent may be in the range of about 1 mg of entrectinib:about 1 to about 1000 μl of solvent, such as about 1 mg of entrectinib:about 1 to about 500 μl of solvent, for example about 1 mg of entrectinib about 5 to about 150 μl of solvent.

The entrectinib may be dissolved in the first solvent at ambient temperature or less. In one embodiment, the contacting step may be carried out at one or more temperatures in the range of ≥about 0° C. to about ≤25° C. In some embodiments, the contacting step is carried out at one or more temperatures ≥about 1° C. In some embodiments, the contacting step is carried out at one or more temperatures ≥about 2° C. In some embodiments, the contacting step is carried out at one or more temperatures ≥about 3° C. In some embodiments, the contacting step is carried out at one or more temperatures ≥about 4° C. In some embodiments, the contacting step is carried out at one or more temperatures ≥about 5° C. In some embodiments, the contacting step is carried out at one or more temperatures ≤about 20° C. In some embodiments, the contacting step is carried out at one or more temperatures ≤about 15° C. In some embodiments, the contacting step is carried out at one or more temperatures ≤about 10° C. In one embodiment, the contacting step is carried out at one or more temperatures in the range of ≥about 0° C. to ≤about 10° C., for example, about 5° C. In one embodiment, the contacting step may be carried out at about ambient temperature e.g. about 25° C.

Alternatively, the entrectinib may be contacted in the solvent at a temperature greater than ambient i.e. greater than 30° C. and below the boiling point of the reaction mixture. The boiling point of the reaction mixture may vary depending on the pressure under which the contacting step is conducted. In one embodiment, the contacting step is carried out at atmospheric pressure (i.e. 1.0135×10⁵ Pa). In one embodiment, the contacting step may be carried out at one or more temperatures in the range of about 40° C. to about ≤95° C. In some embodiments, the contacting step is carried out at one or more temperatures ≥about 41° C. In some embodiments, the contacting step is carried out at one or more temperatures ≥about 42° C. In some embodiments, the contacting step is carried out at one or more temperatures ≥about 43° C. In some embodiments, the contacting step is carried out at one or more temperatures ≥about 44° C. In some embodiments, the contacting step is carried out at one or more temperatures ≥about 45° C. In some embodiments, the contacting step is carried out at one or more temperatures ≥about 46° C. In some embodiments, the contacting step is carried out at one or more temperatures ≥about 47° C. In some embodiments, the contacting step is carried out at one or more temperatures ≥about 48° C. In some embodiments, the contacting step is carried out at one or more temperatures ≥about 49° C. In some embodiments, the contacting step is carried out at one or more temperatures ≥about 50° C. In some embodiments, the contacting step is carried out at one or more temperatures ≤about 90° C. In some embodiments, the contacting step is carried out at one or more temperatures ≤about 80° C. In some embodiments, the contacting step is carried out at one or more temperatures ≤about 70° C. In some embodiments, the contacting step is carried out at one or more temperatures ≤about 60° C. In some embodiments, the contacting step is carried out at one or more temperatures ≤about 59° C. In some embodiments, the contacting step is carried out at one or more temperatures ≤about 58° C. In some embodiments, the contacting step is carried out at one or more temperatures ≤about 57° C. In some embodiments, the contacting step is carried out at one or more temperatures ≤about 56° C. In some embodiments, the contacting step is carried out at one or more temperatures ≤about 55° C. In some embodiments, the contacting step is carried out at one or more temperatures ≤about 54° C. In some embodiments, the contacting step is carried out at one or more temperatures ≤about 53° C. In some embodiments, the contacting step is carried out at one or more temperatures ≤about 52° C. In some embodiments, the contacting step is carried out at one or more temperatures ≤about 51° C. In one embodiment, the contacting step is carried out at one or more temperatures in the range of ≥about 45° C. to ≤about 95° C. In one embodiment, the contacting step is carried out at one or more temperatures in the range of ≥about 45° C. to ≤about 55° C. In one embodiment, the contacting step is carried out at a temperature of about 50° C.

The dissolution or suspension of entrectinib may be encouraged through the use of an aid such as stirring, shaking and/or sonication. Additional solvent may be added to aid the dissolution or suspension of the entrectinib.

The period of time for which the mixture of entrectinib and solvent is treated at the desired temperature is not particularly limiting. In one embodiment, the period of time may be from about 1 minute to about 24 hours, for example, about 5 minutes.

In step (b), acetonitrile is added to the reaction mixture. The quantity of acetonitrile is not particularly limiting. The w/v ratio of entrectinib to acetonitrile may be in the range of about 1 mg of entrectinib:about 1 to about 1000 μl of acetonitrile, such as about 1 mg of entrectinib:about 1 to about 500 μl of acetonitrile, for example about 1 mg of entrectinib:about 1 to about 100 μl of acetonitrile, e.g. about 5 mg of entrectinib:about 30 μl of acetonitrile. These w/v ratios have been calculated using the mass of entrectinib initially dissolved in the first solvent i.e. the quantity of entrectinib inputted into the process.

After the addition of the acetonitrile, the reaction mixture may be treated at ambient temperature or less as described above in connection with first solvent.

Alternatively, the reaction mixture may be treated at a temperature greater than ambient i.e. greater than 30° C. and below the boiling point of the reaction mixture as described above in connection with the first solvent.

The reaction mixture may be left for a further period of time, e.g. about 1 minute to about 24 hours, such as about 1 hour.

The solution or suspension may then be cooled such that the resulting solution or suspension has a temperature below that of the solution or suspension of step (b). The rate of cooling may be from about 0.05° C./minute to about 2° C./minute, such as about 0.1° C./minute to about 1.5° C./minute, for example about 0.1° C./minute or about 0.3° C./minute. When a solution of entrectinib is cooled, a suspension may eventually be observed.

The solution or suspension may be cooled to ambient temperature or a temperature of less than ambient temperature. In one embodiment, the solution or suspension may be cooled to one or more temperatures in the range of ≥about 0° C. to about ≤20° C. In some embodiments, the solution or suspension is cooled to one or more temperatures ≥about 1° C. In some embodiments, the solution or suspension is cooled to one or more temperatures ≥about 2° C. In some embodiments, the solution or suspension is cooled to one or more temperatures ≥about 3° C. In some embodiments, the solution or suspension is cooled to one or more temperatures ≥about 4° C. In some embodiments, the solution or suspension is cooled to one or more temperatures ≥about 5° C. In some embodiments, the solution or suspension is cooled to one or more temperatures ≤about 15° C. In some embodiments, the solution or suspension is cooled to one or more temperatures ≤about 14° C. In some embodiments, the solution or suspension is cooled to one or more temperatures ≤about 13° C. In some embodiments, the solution or suspension is cooled to one or more temperatures ≤about 12° C. In some embodiments, the solution or suspension may be cooled to one or more temperatures ≤about 11° C. In some embodiments, the solution or suspension is cooled to one or more temperatures ≤about 10° C. In one embodiment, the solution or suspension is cooled to one or more temperatures in the range of about 5° C. to about 10° C.

In step (c), the entrectinib acetonitrile solvate Form E is recovered as a crystalline solid. The crystalline solvate may be recovered by directly by filtering, decanting or centrifuging. If desired, the suspension may be mobilised with additional portions of the solvent prior to recovery of the crystalline solid. Alternatively, a proportion or substantially all of the solvent may be evaporated prior to recovery of the crystalline solid.

Howsoever the crystalline solvate is recovered, the separated solvate may be washed with solvent (e.g. one or more of the solvents described above) and dried. Drying may be performed using known methods, for example, at temperatures in the range of about 10° C. to about 60° C., such as about 20° C. to about 40° C., for example, ambient temperature under vacuum (for example about 1 mbar to about 30 mbar) for about 1 hour to about 24 hours. Alternatively, the crystalline solvate may be left to dry under ambient temperature naturally i.e. without the active application of vacuum. It is preferred that the drying conditions are maintained below the point at which the solvate degrades and so when the solvate is known to degrade within the temperature or pressure ranges given above, the drying conditions should be maintained below the degradation temperature or vacuum.

Steps (a) to (c) may be carried out one or more times (e.g. 1, 2, 3, 4 or 5 times). When steps (a) to (c) are carried out more than once (e.g. 2, 3, 4 or 5 times), step (a) may be optionally seeded with crystalline entrectinib anhydrate Form A or crystalline entrectinib acetonitrile solvate Form E which were previously prepared and isolated by methods described herein.

Alternatively or in addition, when steps (a) to (c) are carried out more than once (e.g. 2, 3, 4 or 5 times), the solution or suspension formed in step (b) may be optionally seeded with crystalline entrectinib anhydrate Form A or crystalline entrectinib acetonitrile solvate Form E (which were previously prepared and isolated by methods described herein).

The crystalline entrectinib acetonitrile solvate Form E formed by the processes described above may be free or substantially free of other polymorphic forms of entrectinib. In certain embodiments, the polymorphic purity of the solvate is ≥90%, ≥91%, ≥92%, ≥93%, ≥94%, ≥95% or higher. In certain embodiments, the polymorphic purity of the solvate is ≥95%. In certain embodiments, the polymorphic purity of the solvate is ≥96%. In certain embodiments, the polymorphic purity of the solvate is ≥97%. In certain embodiments, the polymorphic purity of the solvate is ≥98%. In certain embodiments, the polymorphic purity of the solvate is ≥99%.

### Entrectinib Isopropyl Acetate Solvate

It has been discovered that entrectinib can be prepared in a well-defined and consistently reproducible isopropyl acetate solvate form. Moreover, a reliable and scalable method for producing this solvate form has been developed. The entrectinib polymorph provided by the present invention is useful as an active ingredient in pharmaceutical formulations. In certain embodiments, the crystalline solvate form is purifiable. In certain embodiments and depending on time, temperature and humidity, the crystalline solvate form is stable. In certain embodiments, the crystalline solvate form is easy to isolate and handle. In certain embodiments, the process for preparing the crystalline solvate form is scalable.

The crystalline form described herein may be characterised using a number of methods known to the skilled person in the art, including single crystal X-ray diffraction, X-ray powder diffraction (XRPD), differential scanning calorimetry (DSC), thermal gravimetric analysis (TGA), infrared spectroscopy, Raman spectroscopy, nuclear magnetic resonance (NMR) spectroscopy (including solution and solid-state NMR). The chemical purity may be determined by standard analytical methods, such as thin layer chromatography (TLC), gas chromatography, high performance liquid chromatography (HPLC), and mass spectrometry (MS).

In one aspect, the present invention provides a crystalline form of entrectinib which is crystalline entrectinib isopropyl acetate solvate (designated herein as Form F).

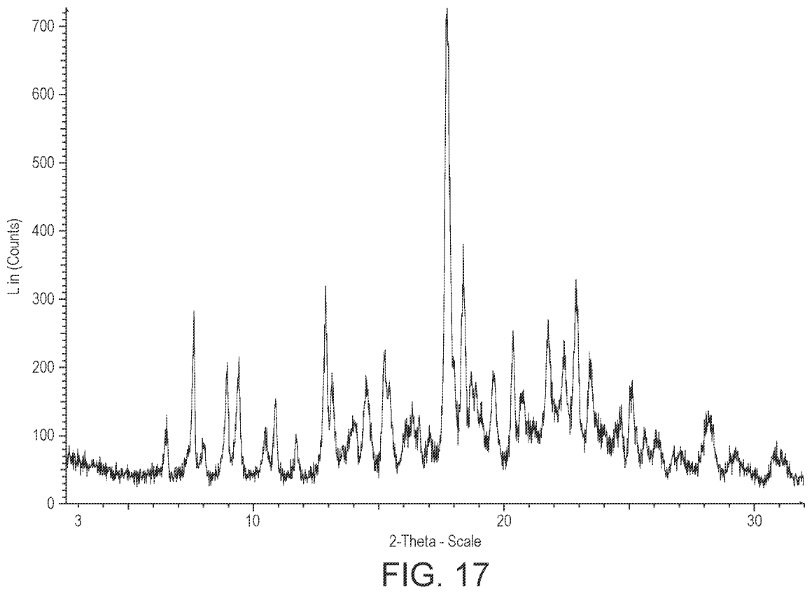

The isopropyl acetate solvate may have an X-ray powder diffraction pattern comprising one or more peaks (for example 1, 2, 3, 4, 5, 6, 7, 8, 9, or 10 peaks) selected from the group consisting of about 6.5, 7.6, 8.0, 8.9, 9.4, 10.5, 10.8, 11.7, 12.9, 13.1, 14.0, 14.5, 15.2, 15.4, 16.1, 16.3, 16.6, 17.0, 17.7, 18.4, 18.7, 18.9, 19.1, 19.6, 20.4, 20.7, 21.8, 22.4, 22.9, 23.5, 24.7, 25.1, 25.7, 26.1, 28.2, and 30.8 degrees two-theta ±0.2 degrees two-theta. In one embodiment, the solvate may have the X-ray powder diffraction pattern substantially as shown in FIG. 17.

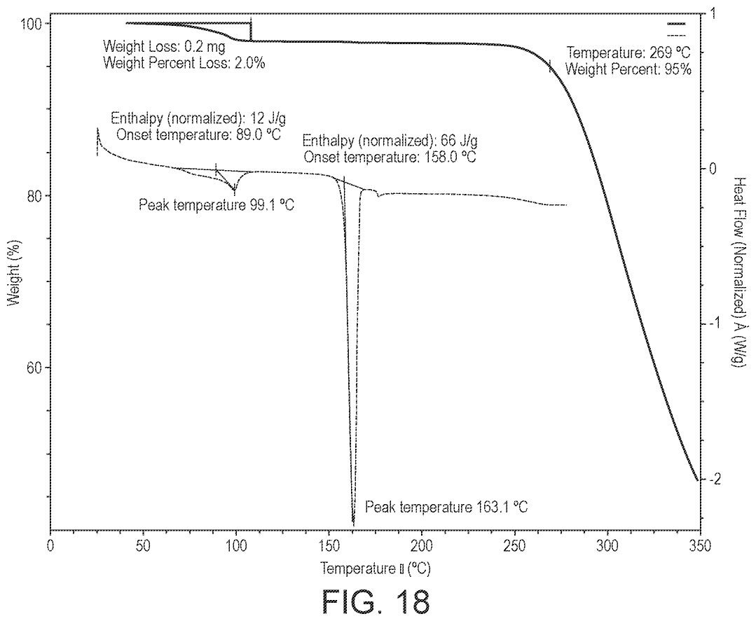

The isopropyl acetate solvate may have a DSC thermogram comprising two endothermic events with onset temperatures of about 89.0° C. and about 158.0° C. In one embodiment, the solvate may have a DSC thermogram substantially as shown in FIG. 18.

The isopropyl acetate solvate may have a TGA thermogram comprising about 2.0% mass loss when heated from about ambient temperature to about 200° C. In one embodiment, the solvate may have a TGA thermogram substantially as shown in FIG. 18.

The crystalline entrectinib isopropyl acetate solvate formed may be free or substantially free of other polymorphic forms of entrectinib. In certain embodiments, the polymorphic purity of the solvate is ≥90%, ≥91%, ≥92%, ≥93%, ≥94%, ≥95% or higher. In certain embodiments, the polymorphic purity of the solvate is ≥95%. In certain embodiments, the polymorphic purity of the solvate is ≥96%. In certain embodiments, the polymorphic purity of the solvate is ≥97%. In certain embodiments, the polymorphic purity of the solvate is ≥98%. In certain embodiments, the polymorphic purity of the solvate is ≥99%.

The crystalline entrectinib isopropyl acetate solvate Form F described above may be prepared by a process comprising the steps of:
(a) contacting entrectinib in a first solvent selected from the group consisting of 1-propanol, 2-propanol, and combinations thereof;
(b) adding isopropyl acetate to the solution of entrectinib; and
(c) recovering entrectinib isopropyl acetate solvate Form F as a crystalline solid.

The quantity of the first solvent is not particularly limiting provided there is enough solvent to dissolve the entrectinib and form a solution. The w/v ratio of entrectinib to the first solvent may be in the range of about 1 mg of entrectinib:about 1 to about 1000 μl of solvent, such as about 1 mg of entrectinib:about 1 to about 500 μl of solvent, for example about 1 mg of entrectinib:about 5 to about 150 μl of solvent.

The entrectinib may be dissolved in the first solvent at ambient temperature or less. In one embodiment, the contacting step may be carried out at one or more temperatures in the range of ≥about 0° C. to about ≤25° C. In some embodiments, the contacting step is carried out at one or more temperatures ≥about 1° C. In some embodiments, the contacting step is carried out at one or more temperatures ≥about 2° C. In some embodiments, the contacting step is carried out at one or more temperatures ≥about 3° C. In some embodiments, the contacting step is carried out at one or more temperatures ≥about 4° C. In some embodiments, the contacting step is carried out at one or more temperatures ≥about 5° C. In some embodiments, the contacting step is carried out at one or more temperatures ≤about 20° C. In some embodiments, the contacting step is carried out at one or more temperatures ≤about 15° C. In some embodiments, the contacting step is carried out at one or more temperatures ≤about 10° C. In one embodiment, the contacting step is carried out at one or more temperatures in the range of ≥about 0° C. to ≤about 10° C., for example, about 5° C. In one embodiment, the contacting step may be carried out at about ambient temperature e.g. about 25° C.

Alternatively, the entrectinib may be contacted in the solvent at a temperature greater than ambient i.e. greater than 30° C. and below the boiling point of the reaction mixture. The boiling point of the reaction mixture may vary depending on the pressure under which the contacting step is conducted. In one embodiment, the contacting step is carried out at atmospheric pressure (i.e. 1.0135×10¹ Pa). In one embodiment, the contacting step may be carried out at one or more temperatures in the range of about 40° C. to about ≤60° C. In some embodiments, the contacting step is carried out at one or more temperatures ≥about 41° C. In some embodiments, the contacting step is carried out at one or more temperatures ≥about 42° C. In some embodiments, the contacting step is carried out at one or more temperatures ≥about 43° C. In some embodiments, the contacting step is carried out at one or more temperatures ≥about 44° C. In some embodiments, the contacting step is carried out at one or more temperatures ≥about 45° C. In some embodiments, the contacting step is carried out at one or more temperatures ≥about 46° C. In some embodiments, the contacting step is carried out at one or more temperatures ≥about 47° C. In some embodiments, the contacting step is carried out at one or more temperatures ≥about 48° C. In some embodiments, the contacting step is carried out at one or more temperatures ≥about 49° C. In some embodiments, the contacting step is carried out at one or more temperatures ≥about 50° C. In some embodiments, the contacting step is carried out at one or more temperatures ≤about 59° C. In some embodiments, the contacting step is carried out at one or more temperatures ≤about 58° C. In some embodiments, the contacting step is carried out at one or more temperatures ≤about 57° C. In some embodiments, the contacting step is carried out at one or more temperatures ≤about 56° C. In some embodiments, the contacting step is carried out at one or more temperatures ≤about 55° C. In some embodiments, the contacting step is carried out at one or more temperatures ≤about 54° C. In some embodiments, the contacting step is carried out at one or more temperatures ≤about 53° C. In some embodiments, the contacting step is carried out at one or more temperatures ≤about 52° C. In some embodiments, the contacting step is carried out at one or more temperatures ≤about 51° C. In one embodiment, the contacting step is carried out at one or more temperatures in the range of ≥about 45° C. to ≤about 55° C. In one embodiment, the contacting step is carried out at a temperature of about 50° C.

The dissolution or suspension of entrectinib may be encouraged through the use of an aid such as stirring, shaking and/or sonication. Additional solvent may be added to aid the dissolution or suspension of the entrectinib.

The period of time for which the mixture of entrectinib and solvent is treated at the desired temperature is not particularly limiting. In one embodiment, the period of time may be from about 1 minute to about 24 minutes, for example, about 5 minutes.

In step (b), isopropyl acetate is added to the reaction mixture. The quantity of isopropyl acetate is not particularly limiting. The w/v ratio of entrectinib to isopropyl acetate may be in the range of about 1 mg of entrectinib:about 1 to about 1000 μl of isopropyl acetate, such as about 1 mg of entrectinib:about 1 to about 500 μl of isopropyl acetate, for example about 1 mg of entrectinib:about 1 to about 100 μl of isopropyl acetate, e.g. about 5 mg of entrectinib:about 30 μl of isopropyl acetate. These w/v ratios have been calculated using the mass of entrectinib initially dissolved in the first solvent i.e. the quantity of entrectinib inputted into the process.

After the addition of the isopropyl acetate, the reaction mixture may be treated at ambient temperature or less as described above in connection with first solvent.

Alternatively, the reaction mixture may be treated at a temperature greater than ambient i.e. greater than 30° C. and below the boiling point of the reaction mixture as described above in connection with the first solvent.

The reaction mixture may be left for a further period of time, e.g. about 1 minute to about 24 hours, such as 1 hour.

The solution or suspension may then be cooled such that the resulting solution or suspension has a temperature below that of the solution or suspension of step (b). The rate of cooling may be from about 0.05° C./minute to about 2° C./minute, such as about 0.1° C./minute to about 1.5° C./minute, for example about 0.1° C./minute. When a solution of entrectinib is cooled, a suspension may eventually be observed.

The solution or suspension may be cooled to ambient temperature or a temperature of less than ambient temperature. In one embodiment, the solution or suspension may be cooled to one or more temperatures in the range of ≥about 0° C. to about ≤20° C. In some embodiments, the solution or suspension is cooled to one or more temperatures ≥about 1° C. In some embodiments, the solution or suspension is cooled to one or more temperatures ≥about 2° C. In some embodiments, the solution or suspension is cooled to one or more temperatures ≥about 3° C. In some embodiments, the solution or suspension is cooled to one or more temperatures ≥about 4° C. In some embodiments, the solution or suspension is cooled to one or more temperatures ≥about 5° C. In some embodiments, the solution or suspension is cooled to one or more temperatures ≤about 15° C. In some embodiments, the solution or suspension is cooled to one or more temperatures ≤about 14° C. In some embodiments, the solution or suspension is cooled to one or more temperatures ≤about 13° C. In some embodiments, the solution or suspension is cooled to one or more temperatures ≤about 12° C. In some embodiments, the solution or suspension may be cooled to one or more temperatures ≤about 11° C. In some embodiments, the solution or suspension is cooled to one or more temperatures ≤about 10° C. In one embodiment, the solution or suspension is cooled to one or more temperatures in the range of about 5° C. to about 10° C.

In step (c), the entrectinib isopropyl acetate solvate Form F is recovered as a crystalline solid. The crystalline solvate may be recovered by directly by filtering, decanting or centrifuging. If desired, the suspension may be mobilised with additional portions of the solvent prior to recovery of the crystalline solid. Alternatively, a proportion or substantially all of the solvent may be evaporated prior to recovery of the crystalline solid.

Howsoever the crystalline solvate is recovered, the separated solvate may be washed with solvent (e.g. one or more of the solvents described above) and dried. Drying may be performed using known methods, for example, at temperatures in the range of about 10° C. to about 60° C., such as about 20° C. to about 40° C., for example, ambient temperature under vacuum (for example about 1 mbar to about 30 mbar) for about 1 hour to about 24 hours. Alternatively, the crystalline solvate may be left to dry under ambient temperature naturally i.e. without the active application of vacuum. It is preferred that the drying conditions are maintained below the point at which the solvate degrades and so when the solvate is known to degrade within the temperature or pressure ranges given above, the drying conditions should be maintained below the degradation temperature or vacuum.

Steps (a) to (c) may be carried out one or more times (e.g. 1, 2, 3, 4 or 5 times). When steps (a) to (c) are carried out more than once (e.g. 2, 3, 4 or 5 times), step (a) may be optionally seeded with crystalline entrectinib isopropyl acetate solvate Form F which were previously prepared and isolated by methods described herein.

Alternatively or in addition, when steps (a) to (c) are carried out more than once (e.g. 2, 3, 4 or 5 times), the solution or suspension formed in step (b) may be optionally seeded with crystalline entrectinib isopropyl acetate solvate Form F (which were previously prepared and isolated by methods described herein).

The crystalline entrectinib isopropyl acetate solvate Form F formed by the processes described above may be free or substantially free of other polymorphic forms of entrectinib. In certain embodiments, the polymorphic purity of the solvate is ≥90%, ≥91%, ≥92%, ≥93%, ≥94%, ≥95% or higher. In certain embodiments, the polymorphic purity of the solvate is ≥95%. In certain embodiments, the polymorphic purity of the solvate is ≥96%. In certain embodiments, the polymorphic purity of the solvate is ≥97%. In certain embodiments, the polymorphic purity of the solvate is ≥98%. In certain embodiments, the polymorphic purity of the solvate is ≥99%.

### Entrectinib Monohydrate

It has been discovered that entrectinib can be prepared in a well-defined and consistently reproducible monohydrate form. Moreover, a reliable and scalable method for producing this monohydrate form has been developed. The entrectinib polymorph provided by the present invention is useful as an active ingredient in pharmaceutical formulations. In certain embodiments, the crystalline monohydrate form is purifiable. In certain embodiments and depending on time, temperature and humidity, the crystalline monohydrate form is stable. In certain embodiments, the crystalline monohydrate form is easy to isolate and handle. In certain embodiments, the process for preparing the crystalline monohydrate form is scalable.

The crystalline form described herein may be characterised using a number of methods known to the skilled person in the art, including single crystal X-ray diffraction, X-ray powder diffraction (XRPD), differential scanning calorimetry (DSC), thermal gravimetric analysis (TGA), infrared spectroscopy, Raman spectroscopy, nuclear magnetic resonance (NMR) spectroscopy (including solution and solid-state NMR). The chemical purity may be determined by standard analytical methods, such as thin layer chromatography (TLC), gas chromatography, high performance liquid chromatography (HPLC), and mass spectrometry (MS).

In one aspect, the present invention provides a crystalline form of entrectinib which is crystalline entrectinib monohydrate (designated herein as Form G).

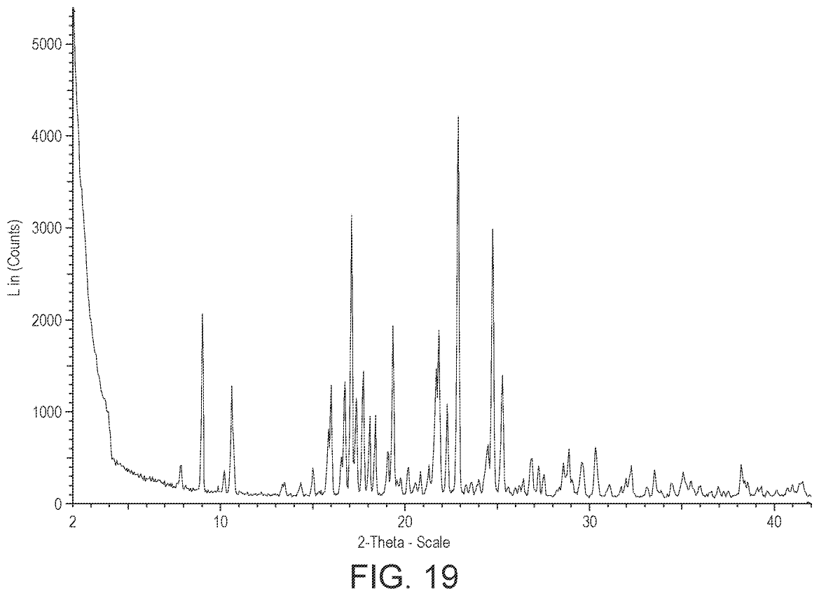

The monohydrate may have an X-ray powder diffraction pattern comprising one or more peaks (for example 1, 2, 3, 4, 5, 6, 7, 8, 9, or 10 peaks) selected from the group consisting of about 7.6, 7.8, 9.0, 9.8, 10.2, 10.6, 13.4, 14.3, 15.0, 15.4, 15.8, 16.0, 16.5, 16.7, 17.1, 17.4, 17.7, 18.0, 18.4, 19.1, 19.3, 19.5, 19.8, 20.2, 20.6, 20.8, 21.3, 21.7, 21.8, 22.3, 22.8, 23.3, 23.6, 24.0, 24.5, 24.8, 25.3, 25.6, 26.0, 26.2, 26.4, 26.9, 27.3, 27.5, 28.2, 28.4, 28.6, 28.9, 29.0, 29.6, and 30.3 degrees two-theta ±0.2 degrees two-theta. In one embodiment, the monohydrate may have the X-ray powder diffraction pattern substantially as shown in FIG. 19.

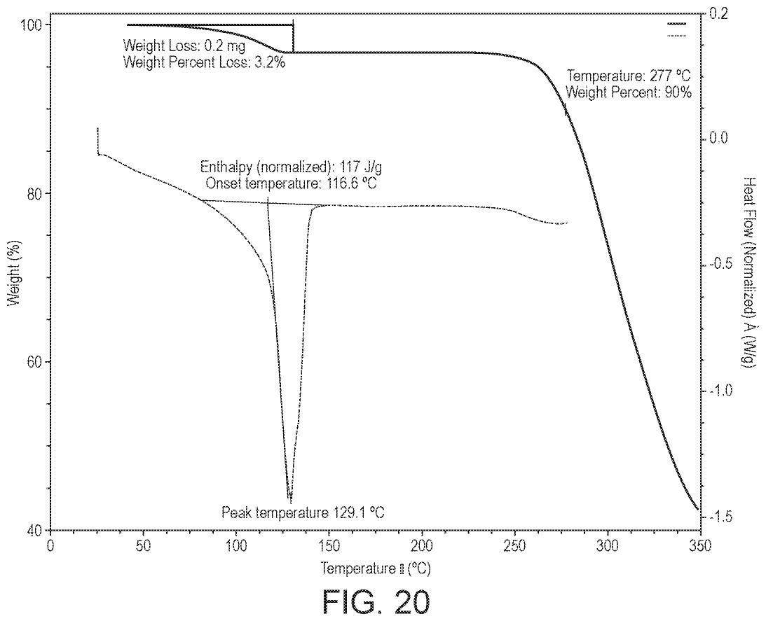

The monohydrate may have a DSC thermogram comprising an endothermic event with an onset temperature of about 116.6° C. In one embodiment, the monohydrate may have a DSC thermogram substantially as shown in FIG. 20.

The monohydrate may have a TGA thermogram comprising about 3.2% mass loss when heated from about ambient temperature to about 200° C. In one embodiment, the monohydrate may have a TGA thermogram substantially as shown in FIG. 20.

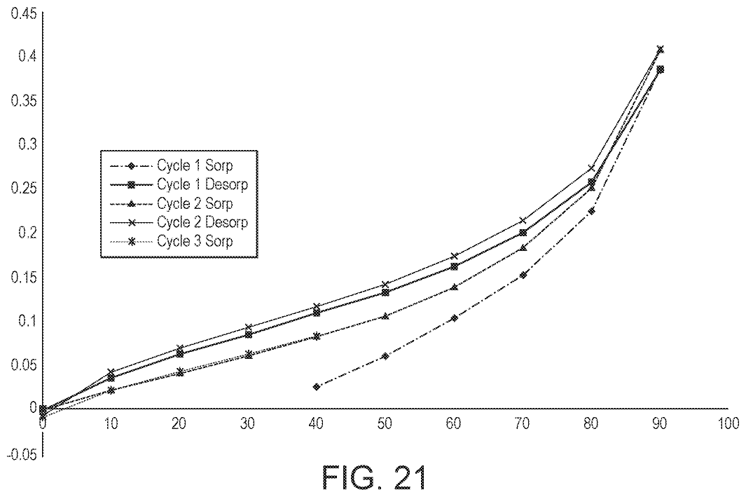

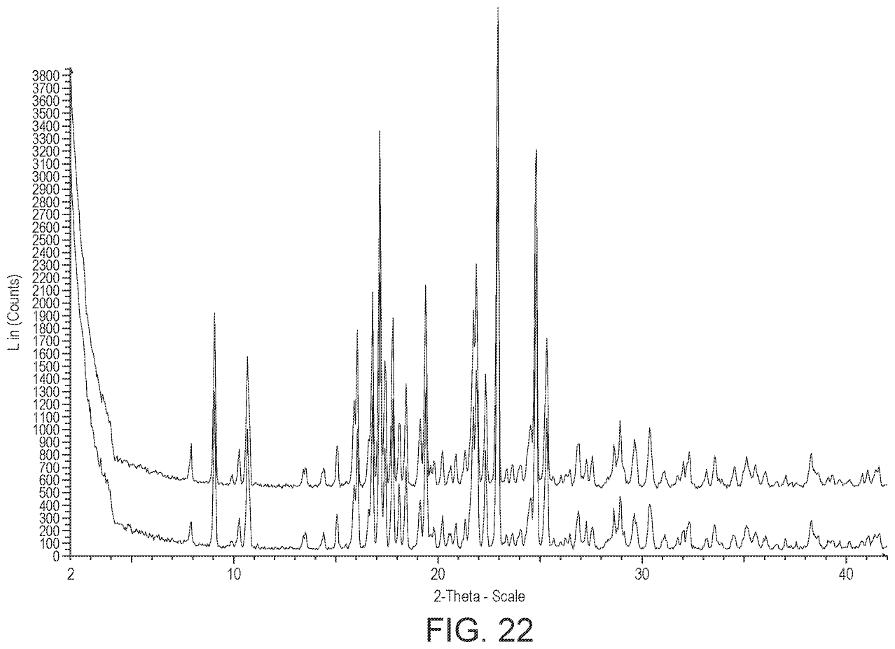

The monohydrate may have a GVS isotherm plot substantially as shown in FIG. 21. The XRPD after analysis showed that the crystalline entrectinib monohydrate was substantially unchanged i.e. there was substantially no change in form after GVS (see FIG. 22).

The crystalline entrectinib monohydrate formed may be free or substantially free of other polymorphic forms of entrectinib. In certain embodiments, the polymorphic purity of the monohydrate is ≥90%, ≥91%, ≥92%, ≥93%, ≥94%, ≥95% or higher. In certain embodiments, the polymorphic purity of the monohydrate is ≥95%. In certain embodiments, the polymorphic purity of the monohydrate is ≥96%. In certain embodiments, the polymorphic purity of the monohydrate is ≥97%. In certain embodiments, the polymorphic purity of the monohydrate is ≥98%. In certain embodiments, the polymorphic purity of the monohydrate is ≥99%.

The crystalline entrectinib monohydrate Form G described above may be prepared by a process comprising the steps of:
(a) contacting entrectinib with propanol (e.g. 1-propanol);
(b) adding water to the solution or suspension of entrectinib; and
(c) recovering entrectinib monohydrate Form G as a crystalline solid.

When the entrectinib is contacted with the propanol, a solution or suspension may form. The quantity of the propanol is not particularly limiting provided there is enough solvent to dissolve the entrectinib and form a solution, or form a suspension of entrectinib in propanol. The w/v ratio of entrectinib to the first solvent may be in the range of about 1 mg of entrectinib:about 1 to about 100 μl of solvent, such as about 1 mg of entrectinib:about 1 to about 50 μl of solvent, for example about 1 mg of entrectinib:about 1 to about 20 μl of solvent, e.g. about 1 mg of entrectinib:about 5 μl of solvent or about 1 mg of entrectinib:about 7.5 μl of solvent.

The entrectinib may be contacted with the propanol at ambient temperature or less. In one embodiment, the contacting step may be carried out at one or more temperatures in the range of ≥about 0° C. to about ≤25° C. In some embodiments, the contacting step is carried out at one or more temperatures ≥about 1° C. In some embodiments, the contacting step is carried out at one or more temperatures ≥about 2° C. In some embodiments, the contacting step is carried out at one or more temperatures ≥about 3° C. In some embodiments, the contacting step is carried out at one or more temperatures ≥about 4° C. In some embodiments, the contacting step is carried out at one or more temperatures ≥about 5° C. In some embodiments, the contacting step is carried out at one or more temperatures ≤about 20° C. In some embodiments, the contacting step is carried out at one or more temperatures ≤about 15° C. In some embodiments, the contacting step is carried out at one or more temperatures ≤about 10° C. In one embodiment, the contacting step is carried out at one or more temperatures in the range of ≥about 0° C. to ≤about 10° C., for example, about 5° C. In one embodiment, the contacting step may be carried out at about ambient temperature e.g. about 25° C.

Alternatively, the entrectinib may be contacted with the propanol at a temperature greater than ambient i.e. greater than 30° C. and below the boiling point of the reaction mixture. The boiling point of the reaction mixture may vary depending on the pressure under which the contacting step is conducted. In one embodiment, the contacting step is carried out at atmospheric pressure (i.e. 1.0135×10¹ Pa). In one embodiment, the contacting step may be carried out at one or more temperatures in the range of about 40° C. to about ≤97° C. In some embodiments, the contacting step is carried out at one or more temperatures ≥about 41° C. In some embodiments, the contacting step is carried out at one or more temperatures ≥about 42° C. In some embodiments, the contacting step is carried out at one or more temperatures ≥about 43° C. In some embodiments, the contacting step is carried out at one or more temperatures ≥about 44° C. In some embodiments, the contacting step is carried out at one or more temperatures ≥about 45° C. In some embodiments, the contacting step is carried out at one or more temperatures ≥about 46° C. In some embodiments, the contacting step is carried out at one or more temperatures ≥about 47° C. In some embodiments, the contacting step is carried out at one or more temperatures ≥about 48° C. In some embodiments, the contacting step is carried out at one or more temperatures ≥about 49° C. In some embodiments, the contacting step is carried out at one or more temperatures ≥about 50° C. In some embodiments, the contacting step is carried out at one or more temperatures ≤about 96° C. In some embodiments, the contacting step is carried out at one or more temperatures ≤about 95° C. In some embodiments, the contacting step is carried out at one or more temperatures ≤about 90° C. In some embodiments, the contacting step is carried out at one or more temperatures ≤about 85° C. In some embodiments, the contacting step is carried out at one or more temperatures ≤about 82° C. In some embodiments, the contacting step is carried out at one or more temperatures ≤about 80° C. In some embodiments, the contacting step is carried out at one or more temperatures ≤about 75° C. In some embodiments, the contacting step is carried out at one or more temperatures ≤about 70° C. In one embodiment, the contacting step is carried out at one or more temperatures in the range of ≥about 45° C. to ≤about 95° C. In one embodiment, the contacting step is carried out at a temperature of about 50° C. In one embodiment, the contacting step is carried out at a temperature of about 70° C.

The temperature of the mixture may change at a rate of heating or cooling from about 0.05° C./minute to about 2° C./minute, such as about 0.1° C./minute to about 1.5° C./minute, for example about 0.3° C./minute.

The dissolution or suspension of entrectinib may be encouraged through the use of an aid such as stirring, shaking and/or sonication. Additional solvent may be added to aid the dissolution or suspension of the entrectinib.

The period of time for which the mixture of entrectinib and solvent is treated at the desired temperature is not particularly limiting. In one embodiment, the period of time may be from about 1 minute to about 24 minutes, for example, about 5 minutes or about 10 minutes.

In step (b), water is added to the reaction mixture. The w/v ratio of entrectinib to water may be in the range of about 1 mg of entrectinib:about 1 to about 1000 μl of water, such as about 1 mg of entrectinib:about 1 to about 100 μl of water, for example about 1 mg of entrectinib:about 1 to about 20 μl of water, e.g. about 1 mg of entrectinib:about 1 to about 10 μl of water. These w/v ratios have been calculated using the mass of entrectinib initially dissolved in the propanol i.e. the quantity of entrectinib inputted into the process.

The water may be added to the reaction in one portion. Alternatively, the water may be added portionwise e.g. dropwise, over a period of time e.g. from about 30 seconds to about 10 minutes, such as about 5 minutes.

The reaction mixture may then be left for a further period of time, e.g. about 1 minute to about 7 days, such as about 1 hour to about 1 day.

The suspension may then be cooled such that the resulting suspension has a temperature below that of the solution or suspension of step (b). The rate of cooling may be from about 0.05° C./minute to about 2° C./minute, such as about 0.1° C./minute to about 1.5° C./minute, for example about 0.1° C./minute or about 0.3° C./minute.

The suspension may be cooled to ambient temperature or a temperature of less than ambient temperature. In one embodiment, the suspension may be cooled to one or more temperatures in the range of ≥about 0° C. to about ≤20° C. In some embodiments, the suspension is cooled to one or more temperatures ≥about 1° C. In some embodiments, the suspension is cooled to one or more temperatures ≥about 2° C. In some embodiments, the suspension is cooled to one or more temperatures ≥about 3° C. In some embodiments, the suspension is cooled to one or more temperatures ≥about 4° C. In some embodiments, the suspension is cooled to one or more temperatures about 5° C. In some embodiments, the suspension is cooled to one or more temperatures ≤about 15° C. In some embodiments, the suspension is cooled to one or more temperatures ≤about 14° C. In some embodiments, the suspension is cooled to one or more temperatures ≤about 13° C. In some embodiments, the suspension is cooled to one or more temperatures ≤about 12° C. In some embodiments, the suspension may be cooled to one or more temperatures ≤about 11° C. In some embodiments, the suspension is cooled to one or more temperatures ≤about 10° C. In one embodiment, the suspension is cooled to one or more temperatures in the range of about 5° C. to about 10° C.

The suspension may be held at this temperature for a period of time, e.g. about 1 minute to about 7 days, such as about 5 minutes to about 5 days. The suspension may be optionally stirred during this time.

In step (c), the entrectinib monohydrate Form G is recovered as a crystalline solid. The crystalline monohydrate may be recovered by directly by filtering, decanting or centrifuging. If desired, the suspension may be mobilised with additional portions of the solvent prior to recovery of the crystalline solid. Alternatively, a proportion or substantially all of the solvent may be evaporated prior to recovery of the crystalline solid.

Howsoever the crystalline monohydrate is recovered, the separated monohydrate may be washed with solvent (e.g. one or more of the solvents described above) and dried. Drying may be performed using known methods, for example, at temperatures in the range of about 10° C. to about 60° C., such as about 20° C. to about 40° C., for example, ambient temperature under vacuum (for example about 1 mbar to about 30 mbar) for about 1 hour to about 24 hours. Alternatively, the crystalline monohydrate may be left to dry under ambient temperature naturally i.e. without the active application of vacuum. It is preferred that the drying conditions are maintained below the point at which the monohydrate degrades and so when the monohydrate is known to degrade within the temperature or pressure ranges given above, the drying conditions should be maintained below the degradation temperature or vacuum.

Steps (a) to (c) may be carried out one or more times (e.g. 1, 2, 3, 4 or 5 times). When steps (a) to (c) are carried out more than once (e.g. 2, 3, 4 or 5 times), step (a) may be optionally seeded with crystalline entrectinib monohydrate Form G which was previously prepared and isolated by a method described herein).

Alternatively or in addition, when steps (a) to (c) are carried out more than once (e.g. 2, 3, 4 or 5 times), the solution or suspension formed in step (b) may be optionally seeded with crystalline entrectinib monohydrate Form G (which was previously prepared and isolated by a method described herein). If seeds are found to persist after addition to the reaction mixture, additional water may be added as described above. The volume of water is not particularly limiting when it is added to the reaction mixture in sufficient quantities to act as an anti-solvent i.e. precipitate more Form G from the reaction mixture.

The crystalline entrectinib monohydrate Form G described above may be prepared by a process comprising the steps of:
(a) contacting entrectinib with a solvent mixture of propanol (e.g. 1-propanol or 2-propanol) and water;
(b) optionally adding water to the solution or suspension of entrectinib; and
(c) recovering entrectinib monohydrate Form G as a crystalline solid.

When the entrectinib is contacted with the solvent mixture, a solution or suspension may form. The entrectinib dissolved or suspended in the solvent mixture may be any solid state form of entrectinib, such as amorphous entrectinib or other polymorphic form.

The volume/volume (v/v) ratio of water to propanol in step (a) may be in the range of about 1 volume of water:about 1 to 100 volumes of propanol, such as about 1 volume of water:about 5 to about 50 volumes of propanol, for example, about 1 volume of water:about 7 to about 40 volumes of propanol.

The quantity of the solvent mixture is not particularly limiting provided there is enough solvent to dissolve the entrectinib and form a solution, or suspend the entrectinib in the solvent mixture. The w/v ratio of entrectinib to the solvent mixture may be in the range of about 1 mg of entrectinib:about 1 to about 100 μl of solvent mixture, such as about 1 mg of entrectinib:about 1 to about 50 μl of solvent mixture, for example about 1 mg of entrectinib:about 1 to about 20 μl of solvent mixture, e.g. about 1 mg of entrectinib:about 2.5 μl, about 5 μl, or about 6 μl of solvent mixture, or about 1 mg of entrectinib:about 7.5 μl of solvent mixture.

The entrectinib may be contacted with the solvent mixture at ambient temperature or less. In one embodiment, the contacting step may be carried out at one or more temperatures in the range of about 0° C. to about ≤25° C. In some embodiments, the contacting step is carried out at one or more temperatures ≥about 1° C. In some embodiments, the contacting step is carried out at one or more temperatures ≥about 2° C. In some embodiments, the contacting step is carried out at one or more temperatures ≥about 3° C. In some embodiments, the contacting step is carried out at one or more temperatures ≥about 4° C. In some embodiments, the contacting step is carried out at one or more temperatures ≥about 5° C. In some embodiments, the contacting step is carried out at one or more temperatures ≤about 20° C. In some embodiments, the contacting step is carried out at one or more temperatures ≤about 15° C. In some embodiments, the contacting step is carried out at one or more temperatures ≤about 10° C. In one embodiment, the contacting step is carried out at one or more temperatures in the range of ≥about 0° C. to ≤about 10° C., for example, about 5° C. In one embodiment, the contacting step may be carried out at about ambient temperature e.g. about 25° C.

Alternatively, the entrectinib may be contacted with the solvent mixture at a temperature greater than ambient i.e. greater than 30° C. and below the boiling point of the reaction mixture. The boiling point of the reaction mixture may vary depending on the pressure under which the contacting step is conducted. In one embodiment, the contacting step is carried out at atmospheric pressure (i.e. 1.0135×10⁵ Pa). In one embodiment, the contacting step may be carried out at one or more temperatures in the range of ≥about 40° C. to about ≤97° C. In some embodiments, the contacting step is carried out at one or more temperatures ≥about 41° C. In some embodiments, the contacting step is carried out at one or more temperatures ≥about 42° C. In some embodiments, the contacting step is carried out at one or more temperatures ≥about 43° C. In some embodiments, the contacting step is carried out at one or more temperatures ≥about 44° C. In some embodiments, the contacting step is carried out at one or more temperatures ≥about 45° C. In some embodiments, the contacting step is carried out at one or more temperatures ≥about 46° C. In some embodiments, the contacting step is carried out at one or more temperatures ≥about 47° C. In some embodiments, the contacting step is carried out at one or more temperatures ≥about 48° C. In some embodiments, the contacting step is carried out at one or more temperatures ≥about 49° C. In some embodiments, the contacting step is carried out at one or more temperatures ≥about 50° C. In some embodiments, the contacting step is carried out at one or more temperatures ≤about 96° C. In some embodiments, the contacting step is carried out at one or more temperatures ≤about 95° C. In some embodiments, the contacting step is carried out at one or more temperatures ≤about 90° C. In some embodiments, the contacting step is carried out at one or more temperatures ≤about 85° C. In some embodiments, the contacting step is carried out at one or more temperatures ≤about 82° C. In some embodiments, the contacting step is carried out at one or more temperatures ≤about 80° C. In some embodiments, the contacting step is carried out at one or more temperatures ≤about 75° C. In some embodiments, the contacting step is carried out at one or more temperatures ≤about 70° C. In one embodiment, the contacting step is carried out at one or more temperatures in the range of ≥about 45° C. to ≤about 95° C. In one embodiment, the contacting step is carried out at a temperature of about 50° C. In one embodiment, the contacting step is carried out at a temperature of about 70° C.

The temperature of the reaction mixture may change at a rate of heating or cooling from about 0.05° C./minute to about 2° C./minute, such as about 0.1° C./minute to about 1.5° C./minute, for example about 0.3° C./minute.

The dissolution or suspension of entrectinib may be encouraged through the use of an aid such as stirring, shaking and/or sonication. Additional solvent mixture may be added to aid the dissolution or suspension of the entrectinib. If the solvent mixture refluxes at the desired temperature selected for the dissolving step (e.g. about 70° C.) and solid collects at or above the solvent mixture line in the reaction vessel, the solid may be added back into reaction mixture.

The period of time for which the mixture of entrectinib and solvent is treated at the desired temperature is not particularly limiting. In one embodiment, the period of time may be from about 1 minute to about 5 days, for example, from about 20 minutes or about 3 days.

Step (b) is optional. In step (b), water may be added to the reaction mixture in order to form a environment which is capable of sustaining seeds (for example, if the seeds are later added). In this respect, a supersaturated solution may form once the water has been added to the reaction mixture. The w/v ratio of entrectinib to water may be in the range of about 1 mg of entrectinib:about 1 to about 1000 μl of water, such as about 1 mg of entrectinib:about 1 to about 100 μl of water, for example about 1 mg of entrectinib:about 1 to about 20 μl of water, e.g. about 1 mg of entrectinib:about 1 to about 10 μl of water. These w/v ratios have been calculated using the mass of entrectinib initially dissolved in the solvent mixture i.e. the quantity of entrectinib inputted into the process.

The water may be added to the reaction in one portion. Alternatively, the water may be added portionwise, for example dropwise, over a period of time e.g. from about 30 seconds to about 30 minutes, such as about 10 minutes.

The reaction mixture may then be left for a further period of time, e.g. about 1 minute to about 7 days, such as about 5 minutes to about 1 day.

The reaction mixture may then be heated at one or more temperatures in the range of ≥about 40° C. to about ≤97° C. In some embodiments, the reaction mixture may be heated at one or more temperatures ≥about 41° C. In some embodiments, the reaction mixture may be heated at one or more temperatures ≥about 42° C. In some embodiments, the reaction mixture may be heated at one or more temperatures ≥about 43° C. In some embodiments, the reaction mixture may be heated at one or more temperatures ≥about 44° C. In some embodiments, the reaction mixture may be heated at one or more temperatures ≥about 45° C. In some embodiments, the dissolving step is carried out at one or more temperatures ≥about 46° C. In some embodiments, the reaction mixture may be heated at one or more temperatures ≥about 47° C. In some embodiments, the reaction mixture may be heated at one or more temperatures ≥about 48° C. In some embodiments, the reaction mixture may be heated at one or more temperatures ≥about 49° C. In some embodiments, the reaction mixture may be heated at one or more temperatures ≥about 50° C. In some embodiments, the reaction mixture may be heated at one or more temperatures ≤about 96° C. In some embodiments, the reaction mixture may be heated at one or more temperatures ≤about 95° C. In some embodiments, the reaction mixture may be heated at one or more temperatures ≤about 90° C. In some embodiments, the reaction mixture may be heated at one or more temperatures ≤about 85° C. In some embodiments, the reaction mixture may be heated at one or more temperatures ≤about 82° C. In some embodiments, the reaction mixture may be heated at one or more temperatures ≤about 80° C. In some embodiments, the reaction mixture may be heated at one or more temperatures ≤about 75° C. In some embodiments, the reaction mixture may be heated at one or more temperatures ≤about 70° C. In one embodiment, the reaction mixture may be heated one or more temperatures in the range of ≥about 45° C. to ≤about 95° C. In one embodiment, the reaction mixture may be heated at a temperature of about 50° C. In one embodiment, the reaction mixture may be heated at a temperature of about 70° C.

The temperature of the reaction mixture may change at a rate of heating or cooling from about 0.05° C./minute to about 2° C./minute, such as about 0.1° C./minute to about 1.5° C./minute, for example about 0.3° C./minute.

Additional water may be added to the reaction mixture. Water acts as an anti-solvent when it is added at this stage of the process. The w/v ratio of entrectinib to water may be in the range of about 1 mg of entrectinib:about 1 to about 1000 μl of water, such as about 1 mg of entrectinib:about 1 to about 100 μl of water, for example about 1 mg of entrectinib:about 1 to about 20 μl of water, e.g. about 1 mg of entrectinib:about 1 to about 10 μl of water. These w/v ratios have been calculated using the mass of entrectinib initially dissolved in the solvent mixture i.e. the quantity of entrectinib inputted into the process.

The water may be added to the reaction in one portion. Alternatively, the water may be added portionwise, for example dropwise, over a period of time e.g. from about 30 seconds to about 30 minutes, such as about 10 minutes.

The reaction mixture may then be left for a further period of time, e.g. about 1 minute to about 7 days, such as about 5 minutes to about 1 day.

The suspension may then be cooled such that the resulting suspension has a temperature below that of the solution or suspension of step (b). The rate of cooling may be from about 0.05° C./minute to about 2° C./minute, such as about 0.1° C./minute to about 1.5° C./minute, for example about 0.1° C./minute or about 0.3° C./minute.

The suspension may be cooled to ambient temperature or a temperature of less than ambient temperature. In one embodiment, the suspension may be cooled to one or more temperatures in the range of ≥about 0° C. to about ≤20° C. In some embodiments, the suspension is cooled to one or more temperatures ≥about 1° C. In some embodiments, the suspension is cooled to one or more temperatures ≥about 2° C. In some embodiments, the suspension is cooled to one or more temperatures ≥about 3° C. In some embodiments, the suspension is cooled to one or more temperatures ≥about 4° C. In some embodiments, the suspension is cooled to one or more temperatures ≥about 5° C. In some embodiments, the suspension is cooled to one or more temperatures ≤about 15° C. In some embodiments, the suspension is cooled to one or more temperatures ≤about 14° C. In some embodiments, the suspension is cooled to one or more temperatures ≤about 13° C. In some embodiments, the suspension is cooled to one or more temperatures ≤about 12° C. In some embodiments, the suspension may be cooled to one or more temperatures ≤about 11° C. In some embodiments, the suspension is cooled to one or more temperatures ≤about 10° C. In one embodiment, the suspension is cooled to one or more temperatures in the range of about 5° C. to about 10° C.

The suspension may be held at this temperature for a period of time, e.g. about 1 minute to about 7 days, such as about 5 minutes to about 5 days. The suspension may be optionally stirred during this time.

In step (c), the entrectinib monohydrate Form G is recovered as a crystalline solid. The crystalline monohydrate may be recovered by directly by filtering, decanting or centrifuging. If desired, the suspension may be mobilised with additional portions of the solvent (e.g. 1-propanol, 2-propanol, water or a mixture thereof) prior to recovery of the crystalline solid. Alternatively, a proportion or substantially all of the solvent may be evaporated prior to recovery of the crystalline solid.

Howsoever the crystalline monohydrate is recovered, the separated monohydrate may be washed with solvent (e.g. one or more of the solvents described above) and dried. Drying may be performed using known methods, for example, at temperatures in the range of about 10° C. to about 60° C., such as about 20° C. to about 40° C., for example, ambient temperature under vacuum (for example about 1 mbar to about 30 mbar) for about 1 hour to about 24 hours. Alternatively, the crystalline monohydrate may be left to dry under ambient temperature naturally i.e. without the active application of vacuum. It is preferred that the drying conditions are maintained below the point at which the monohydrate degrades and so when the monohydrate is known to degrade within the temperature or pressure ranges given above, the drying conditions should be maintained below the degradation temperature or vacuum.

Steps (a) to (c) may be carried out one or more times (e.g. 1, 2, 3, 4 or 5 times). When steps (a) to (c) are carried out more than once (e.g. 2, 3, 4 or 5 times), step (a) may be optionally seeded with crystalline entrectinib monohydrate Form G which was previously prepared and isolated by a method described herein).

Alternatively or in addition, when steps (a) to (c) are carried out more than once (e.g. 2, 3, 4 or 5 times), the solution or suspension formed in step (b) may be optionally seeded with crystalline entrectinib monohydrate Form G (which was previously prepared and isolated by a method described herein). If two portions of water are added to the reaction mixture in step (b), the seeds may be added before the first portion of water, between the first and second portions of water, or after the second portion of water.

The crystalline entrectinib monohydrate Form G formed by the processes described above may be free or substantially free of other polymorphic forms of entrectinib. In certain embodiments, the polymorphic purity of the monohydrate is ≥90%, ≥91%, ≥92%, ≥93%, ≥94%, ≥95% or higher. In certain embodiments, the polymorphic purity of the monohydrate is ≥95%. In certain embodiments, the polymorphic purity of the monohydrate is ≥96%. In certain embodiments, the polymorphic purity of the monohydrate is ≥97%. In certain embodiments, the polymorphic purity of the monohydrate is ≥98%. In certain embodiments, the polymorphic purity of the monohydrate is ≥99%.

In another aspect, the present invention relates to a pharmaceutical composition comprising crystalline entrectinib monohydrate Form G as described herein and a pharmaceutically acceptable excipient.

In another aspect, the present invention relates to a method for treating cancer in a patient comprising administering a therapeutically effective amount of crystalline entrectinib monohydrate Form G as described herein to the patient. The method of treatment includes the treatment of neuroblastoma.

In another aspect, the present invention relates to crystalline entrectinib anhydrate Form G as described herein for use in treating cancer, such as the treatment of neuroblastoma.

Entrectinib 2-methyl THF Solvate

It has been discovered that entrectinib can be prepared in a well-defined and consistently reproducible 2-methyl tetrahydrofuran (2-methyl THF) solvate form. Moreover, a reliable and scalable method for producing this solvate form has been developed. The entrectinib polymorph provided by the present invention is useful as an intermediate to form an entrectinib active ingredient in pharmaceutical formulations. In certain embodiments, the crystalline solvate form is purifiable. In certain embodiments and depending on time, temperature and humidity, the crystalline solvate form is stable. In certain embodiments, the crystalline solvate form is easy to isolate and handle. In certain embodiments, the process for preparing the crystalline solvate form is scalable.

The crystalline form described herein may be characterised using a number of methods known to the skilled person in the art, including single crystal X-ray diffraction, X-ray powder diffraction (XRPD), differential scanning calorimetry (DSC), thermal gravimetric analysis (TGA), infrared spectroscopy, Raman spectroscopy, nuclear magnetic resonance (NMR) spectroscopy (including solution and solid-state NMR). The chemical purity may be determined by standard analytical methods, such as thin layer chromatography (TLC), gas chromatography, high performance liquid chromatography (HPLC), and mass spectrometry (MS).

In one aspect, the present invention provides a crystalline form of entrectinib which is crystalline entrectinib 2-methyl THF solvate (designated herein as Form H). The molar equivalent of entrectinib to 2-methyl THF may be in the range of about 1 molar equivalent of entrectinib:about 0.4 to about 1.2 molar equivalents of 2-methyl THF, for example, about 1 molar equivalent of entrectinib:about 0.5 to about 1 molar equivalents of 2-methyl THF. In one embodiment, the molar equivalent of entrectinib to 2-methyl THF may be about 1 molar equivalent of entrectinib:about 0.5 molar equivalent of 2-methyl THF.

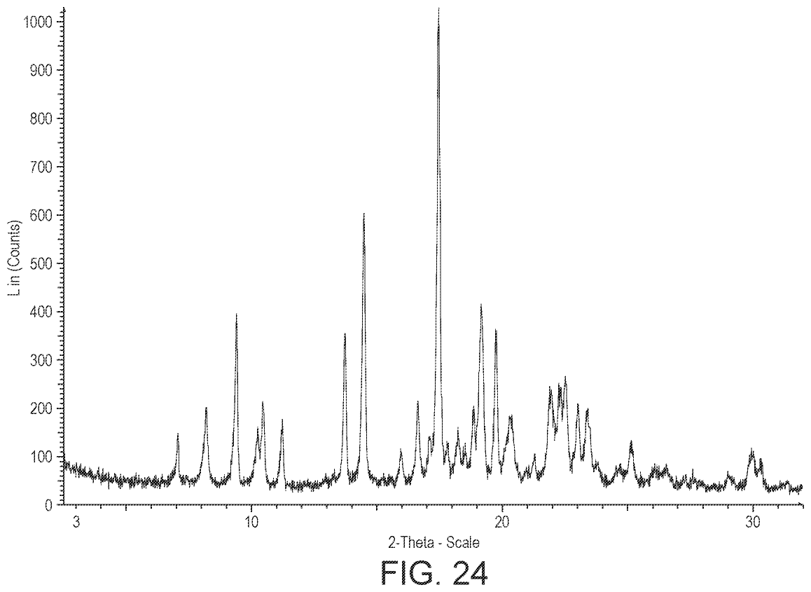

The 2-methyl THF solvate may have an X-ray powder diffraction pattern comprising one or more peaks (for example 1, 2, 3, 4, 5, 6, 7, 8, 9, or 10 peaks) selected from the group consisting of about 7.0, 8.2, 9.4, 10.2, 10.4, 11.2, 13.7, 14.5, 16.0, 16.6, 17.1, 17.5, 17.8, 18.2, 18.5, 18.9, 19.2, 19.8, 20.3, 21.3, 21.9, 22.3, 22.5, 23.0, 23.4, 23.8, 24.7, 25.2, 26.5, 27.3, 29.0, 30.0, and 30.3 degrees two-theta ±0.2 degrees two-theta. In one embodiment, the solvate may have the X-ray powder diffraction pattern substantially as shown in FIG. 24.

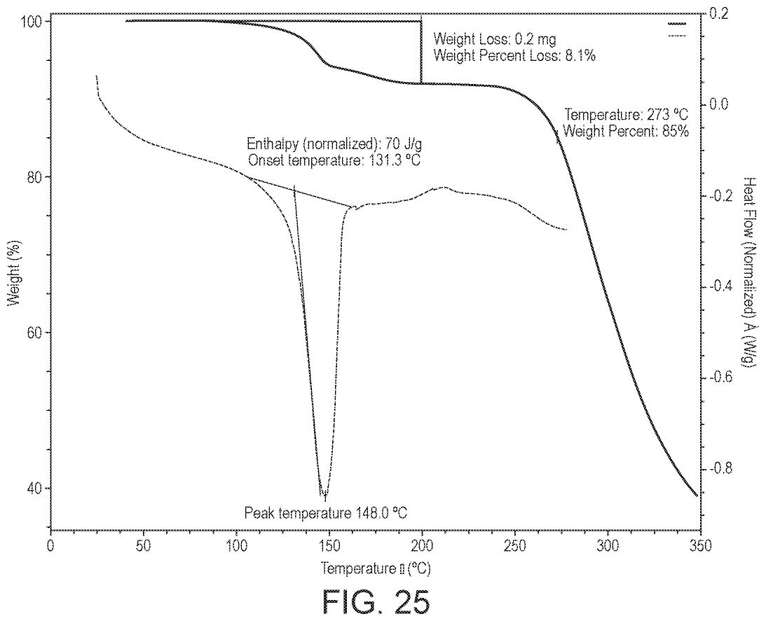

The 2-methyl THF solvate may have a DSC thermogram comprising an endothermic event with an onset temperature of about 131.3° C. In one embodiment, the solvate may have a DSC thermogram substantially as shown in FIG. 25.

The 2-methyl THF solvate may have a TGA thermogram comprising about 8.1% mass loss when heated from about ambient temperature to about 200° C. In one embodiment, the solvate may have a TGA thermogram substantially as shown in FIG. 25.

The crystalline entrectinib 2-methyl THF solvate formed may be free or substantially free of other polymorphic forms of entrectinib. In certain embodiments, the polymorphic purity of the solvate is ≥90%, ≥91%, ≥92%, ≥93%, ≥94%, ≥95% or higher. In certain embodiments, the polymorphic purity of the solvate is ≥95%. In certain embodiments, the polymorphic purity of the solvate is ≥96%. In certain embodiments, the polymorphic purity of the solvate is ≥97%. In certain embodiments, the polymorphic purity of the solvate is ≥98%. In certain embodiments, the polymorphic purity of the solvate is ≥99%.

The crystalline entrectinib 2-methyl THF solvate described above may be prepared by a process comprising the steps of:
(a) contacting entrectinib with a solvent which is 2-methyl tetrahydrofuran;
(b) forming a solution of entrectinib in the solvent; and
(c) recovering entrectinib 2-methyl THF solvate as a crystalline solid.

The quantity of 2-methyl THF solvent is not particularly limiting provided there is enough solvent to dissolve the entrectinib and form a solution. The w/v ratio of entrectinib to solvent may be in the range of about 1 mg of entrectinib:about 1 to about 1000 μl of solvent, such as about 1 mg of entrectinib:about 1 to about 500 μl of solvent, for example about 1 mg of entrectinib:about 1 to about 150 μl of solvent, e.g. about 1 mg of entrectinib:about 1 to about 100 μl of solvent. In one embodiment, the w/v ratio of entrectinib to solvent may be in the range of about 1 mg of entrectinib:about 1 to about 10 μl of solvent.

The entrectinib may be contacted with the solvent at ambient temperature or less. In one embodiment, the contacting step may be carried out at one or more temperatures in the range of ≥about 0° C. to about ≤25° C. In some embodiments, the contacting step is carried out at one or more temperatures ≥about 1° C. In some embodiments, the contacting step is carried out at one or more temperatures ≥about 2° C. In some embodiments, the contacting step is carried out at one or more temperatures ≥about 3° C. In some embodiments, the contacting step is carried out at one or more temperatures ≥about 4° C. In some embodiments, the contacting step is carried out at one or more temperatures ≥about 5° C. In some embodiments, the contacting step is carried out at one or more temperatures ≤about 20° C. In some embodiments, the contacting step is carried out at one or more temperatures ≤about 15° C. In some embodiments, the contacting step is carried out at one or more temperatures ≤about 10° C. In one embodiment, the contacting step is carried out at one or more temperatures in the range of ≥about 0° C. to ≤about 10° C., for example, about 5° C. In one embodiment, the contacting step may be carried out at ambient temperature e.g. about 25° C.

Alternatively, the entrectinib may be contacted with the solvent at a temperature greater than ambient i.e. greater than 30° C. and below the boiling point of the reaction mixture. The boiling point of the reaction mixture may vary depending on the pressure under which the contacting step is conducted. In one embodiment, the contacting step is carried out at atmospheric pressure (i.e. 1.0135×10⁵ Pa). In one embodiment, the contacting step may be carried out at one or more temperatures in the range of ≥about 40° C. to about ≤60° C. In some embodiments, the contacting step is carried out at one or more temperatures ≥about 41° C. In some embodiments, the contacting step is carried out at one or more temperatures ≥about 42° C. In some embodiments, the contacting step is carried out at one or more temperatures ≥about 43° C. In some embodiments, the contacting step is carried out at one or more temperatures ≥about 44° C. In some embodiments, the contacting step is carried out at one or more temperatures ≥about 45° C. In some embodiments, the contacting step is carried out at one or more temperatures ≥about 46° C. In some embodiments, the contacting step is carried out at one or more temperatures ≥about 47° C. In some embodiments, the contacting step is carried out at one or more temperatures ≥about 48° C. In some embodiments, the contacting step is carried out at one or more temperatures ≥about 49° C. In some embodiments, the contacting step is carried out at one or more temperatures ≥about 50° C. In some embodiments, the contacting step is carried out at one or more temperatures ≤about 59° C. In some embodiments, the contacting step is carried out at one or more temperatures ≤about 58° C. In some embodiments, the contacting step is carried out at one or more temperatures ≤about 57° C. In some embodiments, the contacting step is carried out at one or more temperatures ≤about 56° C. In some embodiments, the contacting step is carried out at one or more temperatures ≤about 55° C. In some embodiments, the contacting step is carried out at one or more temperatures ≤about 54° C. In some embodiments, the contacting step is carried out at one or more temperatures ≤about 53° C. In some embodiments, the contacting step is carried out at one or more temperatures ≤about 52° C. In some embodiments, the contacting step is carried out at one or more temperatures ≤about 51° C. In one embodiment, the contacting step is carried out at one or more temperatures in the range of ≥about 45° C. to ≤about 55° C. In one embodiment, the contacting step is carried out at a temperature of about 50° C.

The dissolution of entrectinib may be encouraged through the use of an aid such as stirring, shaking and/or sonication. Additional solvent may be added to aid the dissolution of the entrectinib.

The solution may then be cooled such that the resulting solution has a temperature below that of the solution or suspension of step (b). The rate of cooling may be from about 0.05° C./minute to about 2° C./minute, such as about 0.1° C./minute to about 1.5° C./minute, for example about 0.1° C./minute. When a solution of entrectinib is cooled, a suspension may eventually be observed. When a suspension of entrectinib is cooled, no perceptible change in the appearance of the suspension may occur.

The solution or suspension may be cooled to ambient temperature or a temperature of less than ambient temperature. In one embodiment, the solution or suspension may be cooled to one or more temperatures in the range of ≥about 0° C. to about ≤20° C. In some embodiments, the solution or suspension is cooled to one or more temperatures ≥about 1° C. In some embodiments, the solution or suspension is cooled to one or more temperatures ≥about 2° C. In some embodiments, the solution or suspension is cooled to one or more temperatures ≥about 3° C. In some embodiments, the solution or suspension is cooled to one or more temperatures ≥about 4° C. In some embodiments, the solution or suspension is cooled to one or more temperatures ≥about 5° C. In some embodiments, the solution or suspension is cooled to one or more temperatures ≤about 15° C. In some embodiments, the solution or suspension is cooled to one or more temperatures ≤about 14° C. In some embodiments, the solution or suspension is cooled to one or more temperatures ≤about 13° C. In some embodiments, the solution or suspension is cooled to one or more temperatures ≤about 12° C. In some embodiments, the solution or suspension may be cooled to one or more temperatures ≤about 11° C. In some embodiments, the solution or suspension is cooled to one or more temperatures ≤about 10° C. In one embodiment, the solution or suspension is cooled to one or more temperatures in the range of ≥about 5° C. to about 10° C.

The reaction mixture may then be left for a further period of time, e.g. about 1 minute to about 5 days at the desired temperature as described above. In one embodiment, the reaction mixture was left at a temperature less than ambient temperature for about 3 days.

In step (c), the entrectinib 2-methyl THF solvate is recovered as a crystalline solid. The crystalline solvate may be recovered by directly by filtering, decanting or centrifuging. If desired, the suspension may be mobilised with additional portions of the solvent prior to recovery of the crystalline solid. Alternatively, a proportion or substantially all of the solvent may be evaporated prior to recovery of the crystalline solid.

Howsoever the crystalline solvate is recovered, the separated solvate may be washed with solvent and dried. Drying may be performed using known methods, for example, at temperatures in the range of about 10° C. to about 60° C., such as about 20° C. to about 40° C., for example, ambient temperature under vacuum (for example about 1 mbar to about 30 mbar) for about 1 hour to about 24 hours. Alternatively, the crystalline solvate may be left to dry under ambient temperature naturally i.e. without the active application of vacuum. Alternatively, the crystalline solvate may be left to dry under ambient temperature naturally i.e. without the active application of vacuum. It is preferred that the drying conditions are maintained below the point at which the solvate desolvates and so when the solvate is known to desolvate within the temperature or pressure ranges given above, the drying conditions should be maintained below the degradation temperature or vacuum.

Steps (a) to (c) may be carried out one or more times (e.g. 1, 2, 3, 4 or 5 times). When steps (a) to (c) are carried out more than once (e.g. 2, 3, 4 or 5 times), step (a) may be optionally seeded with crystalline entrectinib 2-methyl THF solvate which was previously prepared and isolated by the first iteration of steps (a) to (c).

Alternatively or in addition, when steps (a) to (c) are carried out more than once (e.g. 2, 3, 4 or 5 times), the solution formed in step (b) may be optionally seeded with crystalline entrectinib 2-methyl THF solvate 0.5 eq. (which was previously prepared and isolated by a method described herein).

### Entrectinib Methanol Solvate

It has been discovered that entrectinib can be prepared in a well-defined and consistently reproducible methanol solvate form. Moreover, a reliable and scalable method for producing this solvate form has been developed. The entrectinib polymorph provided by the present invention is useful as an intermediate to form an entrectinib active ingredient in pharmaceutical formulations. In certain embodiments, the crystalline solvate form is purifiable. In certain embodiments and depending on time, temperature and humidity, the crystalline solvate form is stable. In certain embodiments, the crystalline solvate form is easy to isolate and handle. In certain embodiments, the process for preparing the crystalline solvate form is scalable.

The crystalline form described herein may be characterised using a number of methods known to the skilled person in the art, including single crystal X-ray diffraction, X-ray powder diffraction (XRPD), differential scanning calorimetry (DSC), thermal gravimetric analysis (TGA), infrared spectroscopy, Raman spectroscopy, nuclear magnetic resonance (NMR) spectroscopy (including solution and solid-state NMR). The chemical purity may be determined by standard analytical methods, such as thin layer chromatography (TLC), gas chromatography, high performance liquid chromatography (HPLC), and mass spectrometry (MS).

In one aspect, the present invention provides a crystalline form of entrectinib which is crystalline entrectinib methanol solvate (designated herein as Form J). The molar equivalent of entrectinib to methanol may be in the range of about 1 molar equivalent of entrectinib:about 0.5 to about 1.5 molar equivalents of methanol, for example, about 1 molar equivalent of entrectinib:about 0.7 to about 1.2 molar equivalents of methanol. In one embodiment, the molar equivalent of entrectinib to methanol may be about 1 molar equivalent of entrectinib:about 1 molar equivalent of methanol.

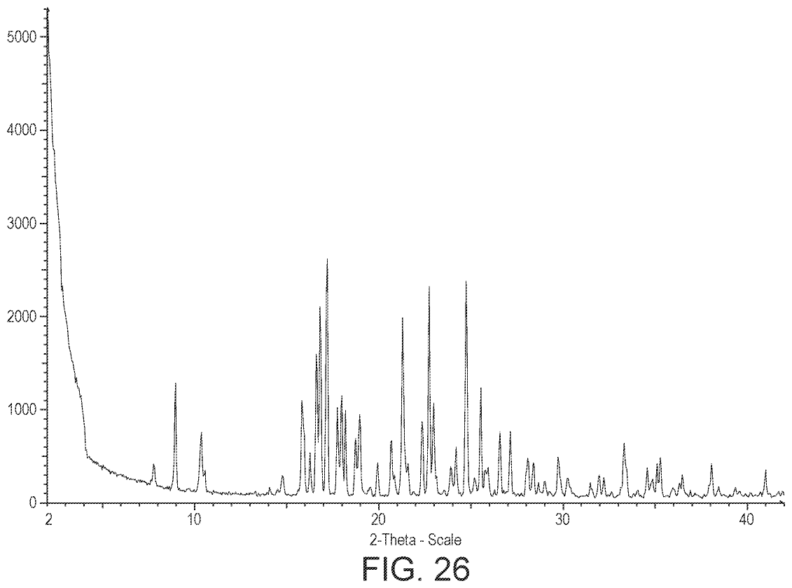

The methanol solvate may have an X-ray powder diffraction pattern comprising one or more peaks (for example 1, 2, 3, 4, 5, 6, 7, 8, 9, or 10 peaks) selected from the group consisting of about 7.8, 8.9, 10.3, 10.5, 14.1, 14.5, 14.8, 15.8, 16.3, 16.6, 16.8, 17.1, 17.7, 17.9, 18.2, 18.7, 19.0, 19.5, 19.9, 20.6, 20.9, 21.3, 21.6, 22.4, 22.7, 22.9, 23.6, 23.9, 24.2, 24.8, 25.2, 25.5, 25.8, 25.9, 26.6, 27.1, 28.1, 28.4, 28.6, 29.0, 29.8, and 30.2 degrees two-theta ±0.2 degrees two-theta. In one embodiment, the solvate may have the X-ray powder diffraction pattern substantially as shown in FIG. 26.

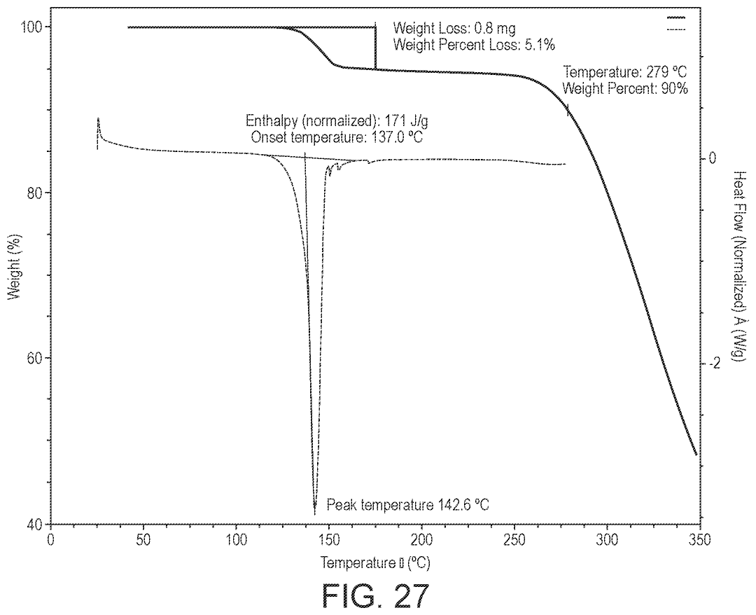

The methanol solvate may have a DSC thermogram comprising an endothermic event with an onset temperature of about 137.0° C. In one embodiment, the solvate may have a DSC thermogram substantially as shown in FIG. 27.

The methanol solvate may have a TGA thermogram comprising about 5.1% mass loss when heated from about ambient temperature to about 200° C. In one embodiment, the solvate may have a TGA thermogram substantially as shown in FIG. 27.

The crystalline entrectinib methanol solvate formed may be free or substantially free of other polymorphic forms of entrectinib. In certain embodiments, the polymorphic purity of the solvate is ≥90%, ≥91%, ≥92%, ≥93%, ≥94%, ≥95% or higher. In certain embodiments, the polymorphic purity of the solvate is ≥95%. In certain embodiments, the polymorphic purity of the solvate is ≥96%. In certain embodiments, the polymorphic purity of the solvate is ≥97%. In certain embodiments, the polymorphic purity of the solvate is ≥98%. In certain embodiments, the polymorphic purity of the solvate is ≥99%.

The crystalline entrectinib methanol solvate described above may be prepared by a process comprising the steps of:
(a) contacting entrectinib with a solvent which is methanol; and
(b) forming a suspension of entrectinib in methanol.

The quantity of methanol is not particularly limiting provided there is enough methanol to substantially suspend the entrectinib. The w/v ratio of entrectinib to methanol solvent may be in the range of about 1 mg of entrectinib:about 1 to about 1000 μl of methanol, such as about 1 mg of entrectinib:about 1 to about 500 μl of methanol, for example about 1 mg of entrectinib:about 1 to about 250 μl of methanol, e.g. about 1 mg of entrectinib:about 5 to about 110 μl of methanol.

The entrectinib may be contacted with methanol at ambient temperature or less. In one embodiment, the contacting step may be carried out at one or more temperatures in the range of ≥about 0° C. to about ≤25° C. In some embodiments, the contacting step is carried out at one or more temperatures ≥about 1° C. In some embodiments, the contacting step is carried out at one or more temperatures ≥about 2° C. In some embodiments, the contacting step is carried out at one or more temperatures ≥about 3° C. In some embodiments, the contacting step is carried out at one or more temperatures ≥about 4° C. In some embodiments, the contacting step is carried out at one or more temperatures ≥about 5° C. In some embodiments, the contacting step is carried out at one or more temperatures ≤about 20° C. In some embodiments, the contacting step is carried out at one or more temperatures ≤about 15° C. In some embodiments, the contacting step is carried out at one or more temperatures ≤about 10° C. In one embodiment, the contacting step is carried out at one or more temperatures in the range of ≥about 0° C. to ≤about 10° C., for example, about 5° C. In one embodiment, the contacting step may be carried out at ambient temperature e.g. about 25° C.

Alternatively, the entrectinib may be contacted with methanol at a temperature greater than ambient i.e. greater than 30° C. and below the boiling point of the reaction mixture. The boiling point of the reaction mixture may vary depending on the pressure under which the contacting step is conducted. Methanol has a boiling point of about 64.7° C. at atmospheric pressure (i.e. 1.0135×10⁵ Pa). In one embodiment, the contacting step may be carried out at one or more temperatures in the range of ≥about 30° C. to about ≤70° C. In some embodiments, the contacting step is carried out at one or more temperatures ≥about 41° C. In some embodiments, the contacting step is carried out at one or more temperatures ≥about 42° C. In some embodiments, the contacting step is carried out at one or more temperatures ≥about 43° C. In some embodiments, the contacting step is carried out at one or more temperatures ≥about 44° C. In some embodiments, the contacting step is carried out at one or more temperatures ≥about 45° C. In some embodiments, the contacting step is carried out at one or more temperatures ≥about 46° C. In some embodiments, the contacting step is carried out at one or more temperatures ≥about 47° C. In some embodiments, the contacting step is carried out at one or more temperatures ≥about 48° C. In some embodiments, the contacting step is carried out at one or more temperatures ≥about 49° C. In some embodiments, the contacting step is carried out at one or more temperatures ≥about 50° C. In some embodiments, the contacting step is carried out at one or more temperatures ≤about 70° C. In some embodiments, the contacting step is carried out at one or more temperatures ≤about 69° C. In some embodiments, the contacting step is carried out at one or more temperatures ≤about 68° C. In some embodiments, the contacting step is carried out at one or more temperatures ≤about 67° C. In some embodiments, the contacting step is carried out at one or more temperatures ≤about 66° C. In some embodiments, the contacting step is carried out at one or more temperatures ≤about 65° C. In some embodiments, the contacting step is carried out at one or more temperatures ≤about 64° C. In some embodiments, the contacting step is carried out at one or more temperatures ≤about 63° C. In some embodiments, the contacting step is carried out at one or more temperatures ≤about 62° C. In some embodiments, the contacting step is carried out at one or more temperatures ≤about 61° C. In one embodiment, the contacting step is carried out at one or more temperatures in the range of ≥about 45° C. to ≤about 65° C. In one embodiment, the contacting step is carried out at a temperature of about 50° C. In another embodiment, the contacting step is carried out at a temperature of about 60° C.

The period of time for which the mixture of entrectinib and solvent is treated at the desired temperature is not particularly limiting. In one embodiment, the period of time may be from about 1 minute to about 7 days, for example, about 3 days.

Alternatively, entrectinib may be contacted with methanol at ambient temperature or less and then matured between this temperature and one or more temperatures greater than ambient temperature. Ambient temperature, temperatures less than ambient, and temperatures greater than ambient are as described above. The maturation step may comprise oscillating the temperature for a period of time (e.g. about 4 hours) at ambient, a period of time at the temperature greater than ambient (e.g. about 4 hours), followed by another period of time (e.g. about 4 hours) at ambient, and so on, for an extended period of time (e.g. about 6 days).

The suspension of entrectinib may be agitated through the use of an aid such as stirring, shaking and/or sonication.

The process may further comprise the step of recovering entrectinib methanol solvate as a crystalline solid. The crystalline solvate may be recovered by directly by filtering, decanting or centrifuging. If desired, the suspension may be mobilised with additional portions of methanol prior to recovery of the crystalline solid. Alternatively, a proportion or substantially all of the methanol solvent may be evaporated prior to recovery of the crystalline solid.

Howsoever the crystalline methanol solvate is recovered, the separated solvate may be washed with solvent and dried. Drying may be performed using known methods, for example, at temperatures in the range of about 10° C. to about 60° C., such as about 20° C. to about 40° C., for example, ambient temperature under vacuum (for example about 1 mbar to about 30 mbar) for about 1 hour to about 24 hours. Alternatively, the crystalline solvate may be left to dry under ambient temperature naturally i.e. without the active application of vacuum. It is preferred that the drying conditions are maintained below the point at which the methanol solvate desolvates and so when the solvate is known to desolvate within the temperature or pressure ranges given above, the drying conditions should be maintained below the desolvation temperature or vacuum.

Embodiments and/or optional features of the invention have been described above. Any aspect of the invention may be combined with any other aspect of the invention, unless the context demands otherwise. Any of the embodiments or optional features of any aspect may be combined, singly or in combination, with any aspect of the invention, unless the context demands otherwise.

The invention will now be described further by reference to the following examples, which are intended to illustrate but not limit, the scope of the invention.

### EXAMPLES

### 1.1 Instrument and Methodology Details

### 1.1 X-Ray Powder Diffraction (XRPD)

### 1.1.1 Bruker AXS D8 Advance

XRPD diffractograms were collected on a Bruker D8 diffractometer using Cu Kα radiation (40 kV, 40 mA) and a θ-2θ goniometer fitted with a Ge monochromator. The incident beam passes through a 2.0 mm divergence slit followed by a 0.2 mm anti-scatter slit and knife edge. The diffracted beam passes through an 8.0 mm receiving slit with 2.5° Soller slits followed by the Lynxeye Detector. The software used for data collection and analysis was Diffrac Plus XRD Commander and Diffrac Plus EVA respectively.

Samples were run under ambient conditions as flat plate specimens using powder as received. The sample was prepared on a polished, zero-background (510) silicon wafer by gently pressing onto the flat surface or packed into a cut cavity. The sample was rotated in its own plane.

The details of the standard data collection method are:
Angular range: 2 to 42° 2θ
Step size: 0.05° 2θ
Collection time: 0.5 s/step (total collection time: 6.40 min)

The instrument is performance checked weekly using NIST1976 corundum to the peak position of 35.149±0.01° 2θ

### 1.1.2 PANalytical Empyrean

XRPD diffractograms were collected on a PANalytical Empyrean diffractometer using Cu Kα radiation (45 kV, 40 mA) in transmission geometry. A 0.5° slit, 4 mm mask and 0.04 rad Soller slits with a focusing mirror were used on the incident beam. A PIXcel^{3D} detector, placed on the diffracted beam, was fitted with a receiving slit and 0.04 rad Soller slits. The software used for data collection was X'Pert Data Collector using X'Pert Operator Interface. The data were analysed and presented using Diffrac Plus EVA or HighScore Plus.

Samples were prepared and analysed on a metal well-plate in transmission mode. X-ray transparent film was used between the metal sheets on the metal well-plate and powders (approximately 1-2 mg) were used as received. The scan mode for the metal plate used the gonio scan axis.

The details of the standard screening data collection method are:
Angular range: 2.5 to 32.0° 2θ
Step size: 0.0130° 2θ
Collection time: 12.75 s/step (total collection time of 2.07 min)

### 1.2 Differential Scanning Calorimetry (DSC)

### 1.2.1 TA Instruments Q2000

DSC data were collected on a TA Instruments Q2000 equipped with a 50 position auto-sampler. Typically, 0.5-3 mg of each sample, in a pin-holed aluminium pan, was heated at 10° C./min from 25° C. to 280° C. A purge of dry nitrogen at 50 ml/min was maintained over the sample.

The instrument control software was Advantage for Q Series and Thermal Advantage and the data were analysed using Universal Analysis or TRIOS.

### 1.2.2 TA Instruments Discovery DSC

DSC data were collected on a TA Instruments Discovery DSC equipped with a 50 position auto-sampler. Typically, 0.5-3 mg of each sample, in a pin-holed aluminium pan, was heated at 10° C./min from 25° C. to 380° C. A purge of dry nitrogen at 50 ml/min was maintained over the sample.

The instrument control software was TRIOS and the data were analysed using TRIOS or Universal Analysis.

### 1.3 Thermo-Gravimetric Analysis (TGA)

TGA data were collected on a TA Instruments Discovery TGA, equipped with a 25 position auto-sampler. Typically, 5-10 mg of each sample was loaded onto a pre-tared aluminium DSC pan and heated at 10° C./min from ambient temperature to 350° C. A nitrogen purge at 25 ml/min was maintained over the sample.

The instrument control software was TRIOS and the data were analysed using TRIOS or Universal Analysis.

### 1.4 Gravimetric Vapour Sorption (GVS)

Hygroscopicity of a solid material may be determined by means of gravimetric vapour sorption (GVS) analysis, sometimes known by dynamic vapour sorption (DVS) analysis. The experiment subjects a sample material which is held in a fine wire basket on a microbalance within a temperature and humidity controlled environment (chamber). Using the software, the collected data can then be processed to determine the isotherm points at the increment ranges specified during the experiment and show the overall water uptake of the material.

Sorption isotherms were obtained using a SMS DVS Intrinsic moisture sorption analyser, controlled by DVS Intrinsic Control software. The sample temperature was maintained at 25° C. by the instrument controls. The humidity was controlled by mixing streams of dry and wet nitrogen, with a total flow rate of 200 ml/min. The relative humidity was measured by a calibrated Rotronic probe (dynamic range of 1.0-100% RH), located near the sample. The weight change, (mass relaxation) of the sample as a function of % RH was constantly monitored by a microbalance (accuracy ±0.005 mg).

Typically, 5-30 mg of sample was placed in a tared mesh stainless steel basket under ambient conditions. The sample was loaded and unloaded at 40% RH and 25° C. (typical room conditions). A moisture sorption isotherm was performed as outlined below (2 scans per complete cycle). The standard isotherm was performed at 25° C. at 10% RH intervals over a 0-90% RH range. Typically, a double cycle (4 scans) was carried out. Data analysis was carried out within Microsoft Excel using the DVS Analysis Suite.

TABLE 1
Method for SMS DVS Intrinsic experiments

| Parameter | Value |
|---|---|
| Adsorption - Scan 1 | 40-90 |
| Desorption, Adsorption - Scan 2 | 90-0, 0-40 |
| Intervals (% RH) | 10 |
| Number of Scans | 4 |
| Flow rate (ml/min) | 200 |
| Temperature (° C.) | 25 |
| Stability (° C./min) | 0.2 |
| Sorption Time (hours) | 6 hour time out |
| Number of cycles | 2 |

### 1.5 Four Hour Solubility

Sufficient sample was suspended in 1.0 ml media for a maximum anticipated concentration of ca. 10 mg/ml of the free form of the compound. The resulting suspensions were then shaken at 25° C./750 rpm for 4 hours. The pH of the sample solutions was checked after 1 hour and adjusted as necessary with 0.2 M NaOH to ensure that the desired pH was maintained throughout (±0.2). After equilibration, the appearance was noted, and the final pH of the saturated solution was measured. Samples were then filtered through a glass C′ fibre filter (Particle retention size 1.2 μm). Samples suspended in pH 1.2 buffer were diluted 10 times with pH 1.2 buffer, all other samples were analysed undiluted.

Quantitation was by HPLC with reference to a standard solution of approximately 0.15 mg/ml. Different volumes of the standard, diluted and undiluted sample solutions were injected. The solubility was calculated using the peak areas determined by integration of the peak found at the same retention time as the principal peak in the standard injection.

TABLE 2
Preparation of solubility media

| | | Final |
|---|---|---|
| Media | Preparation | pH |
| pH 1.2 | 25 ml of a 0.2M KCl solution was mixed with 42.5 | 1.2 |
| Hydrochloric | ml 0.2M HCl in a 100 ml volumetric flask, and | |
| Acid Buffer | then made to volume with deionized water. | |
| pH 2.8 | 0.3 g of Sodium dihydrogen phosphate dissolved in | 2.8 |
| Buffer | deionized water in 50 ml volumetric flask and made | |
| | up to near volume with deionized water. pH was | |
| | adjusted using Phosphoric acid. | |
| pH 4.5 | 0.3 g sodium acetate trihydrate was dissolved in 1.4 | 4.5 |
| Acetate | ml 2M acetic acid in a 500 ml volumetric flask, and | |
| Buffer | then made to volume with deionized water. | |
| pH 6.8 | 25 ml of a 0.2M monobasic potassium phosphate | 6.8 |
| Phosphate | solution was mixed with 11.2 ml 0.2M sodium | |
| Buffer | hydroxide in a 100 ml volumetric flask, and then | |
| | made to volume with deionized water. | |
| pH 7.5 | 25 ml of a 0.2M monobasic potassium phosphate | 7.5 |
| Phosphate | solution was mixed with 19.6 ml 0.2M sodium | |
| Buffer | hydroxide in a 100 ml volumetric flask, and then | |
| | made to volume with deionized water. pH adjusted | |
| | using 0.2M NaOH. | |

TABLE 3
HPLC method for solubility measurements

| Parameter | Value | | |
|---|---|---|---|
| Type of method | Reverse phase with gradient elution | | |
| Assay type | Solubility | | |
| Column | Phenomenex Luna, C18 (2) 5 μm 50 × 4.6 mm | | |
| Column Temperature (° C.) | 25 | Autosampler temperature (° C.) | Ambient |
| Standard Injections (μl) | 1, 2, 3, 4, 5, 7 | | |
| Sample Injections (μl) | 1, 2, 3, 5, 10, 20 | | |
| Detection: | 260, 90 | | |
| Wavelength & Bandwidth (nm) | (Additional suitable wavelengths can be used if desired) | | |
| Flow Rate (ml/min) | 2.0 | | |
| Mobile Phase A | 0.1% TFA in Water | | |
| Mobile Phase B | 0.085% TFA in Acetonitrile | | |
| Timetable | Time (min) | % Phase A | % Phase B |
| | 0.0 | 95.0 | 5.0 |
| | 1.0 | 80.0 | 20.0 |
| | 2.3 | 5.0 | 95.0 |
| | 3.3 | 5.0 | 95.0 |
| | 3.5 | 95.0 | 5.0 |
| | 4.4 | 95.0 | 5.0 |

1.6 Sirius inForm

Data were collected on a Sirius inForm instrument fitted with a dual UV Dip Probe attachment and Ag/AgCl combination pH electrode. The electrode was calibrated using the four plus parameters derived from a blank titration. The base titrant was standardised by titration with TRIS. 0.5 M HCl and NaOH aqueous solutions were used as the acid and base titrants respectively for the testing. Stirring was facilitated by a dual overhead stirrer to allow thorough mixing within the vessel, and media was introduced via a capillary bundle attached to a dispensing bank comprised of six precision dispensing units. A Peltier heating jacket was used to maintain the temperature of the titration vessel. Discs were introduced to the vessel via the tablet picker housed in the probe arm, after the desired temperature of the media had been reached.

Sirius inForm Assay Design, Control and Refine software were used to design, run and refine data respectively.

### 1.6.1 Molar Extinction Coefficient (MEC)

The reference sample was prepared as a 10.1 mM stock solution in DMSO. MEC data was then collected using UV metric method with initial addition of 100 μl of DMSO stock. UV Spectra were then collected from low to high pH (pH 2-12) in two titrations using a 5 mm path length. The MEC data set was then imported into the dissolution data files in order to convert the UV absorbance measured to concentration. The concentration range for UV data collected was 11.58-21.93 μm. MEC data were collected in the dissolution media (36 ml of ISA water+4 ml of GI Tract buffer) and at 37° C. to match the dissolution experiments.

### 1.6.2 Intrinsic Dissolution Rate (IDR)

Ca. 8-20 mg of the sample was compressed in a 3 mm disc recess, under 100 kg for 2 minutes, with greaseproof paper on the compression base, to form non-disintegrating discs. The discs were then plugged with a bung so that only one surface was exposed to the media during analysis and transferred to the Sirius inForm dissolution apparatus. Analysis was performed at 37° C. in 36 ml of ISA water+4 ml of GI Tract buffer for 2 hours in 4 sectors with UV spectra collected every 30 seconds for each sector (Section 1—pH 2.8, Sector 2—pH 5.5, Sector 3—pH 6.5, Sector 4—pH 7.4). A stir speed of 50 rpm was used with a 5 mm path length probe. The IDR was calculated based on the surface area of the 3 mm disc recess used (7.07 mm² surface area).

TABLE 4
Preparation of IDR media

| Media | Preparation | Final pH |
|---|---|---|
| Gastrointestinal | 8.9 g Sodium chloride, 13.6 g sodium acetate trihydrate and | 5.7 |
| Tract Buffer | 13.8 g sodium dihydrogen phosphate monohydrate dissolved in | |
| | 1 L IC grade water. | |

### Abbreviations

DCM dichloromethane
DMF Dimethylformamide
eq. equivalent
IPA isopropanol
MEK methyl ethyl ketone
2-methyl THF 2-methyl tetrahydrofuran
MIBK methyl isobutyl ketone
NMP 1-methyl-2-pyrrolidinone
RT room temperature
% RH percent Relative Humidity
THF tetrahydrofuran

### Form A (Anhydrous)

### Example 1—Form A (Anhydrous)

Entrectinib (38.2 mg) was dissolved in MEK (200 μl, 5 vol) and left to stir for 5 minutes at 50° C. After 5 minutes of stirring, the sample was treated with acetonitrile (1000 μl). The resulting solution was then left at 50° C. for 1 hour before cooling to 5° C. at 0.1° C./min and left overnight. The resulting suspension was filtered and dried under suction before analysis by XRPD.

### Example 2—Form A (Anhydrous)

Entrectinib (38.2 mg) was dissolved in 1-propanol (200 μl, 5 vol) and left to stir for 5 minutes at 50° C. After 5 minutes of stirring, the sample was treated with acetonitrile (1000 μl). The resulting solution was then left at 50° C. for 1 hour before cooling to 5° C. at 0.1° C./min and left overnight. The resulting suspension was filtered and dried under suction before analysis by XRPD.

### Example 3—Form A (Anhydrous)

A sample of Form E (acetonitrile solvate) (Example 43) was dried under vacuum at RT for 1 day.

### Example 4—Form A (Anhydrous)

A sample of Form E (acetonitrile solvate) (Example 44) was dried under vacuum at RT for 1 day.

### Example 5—Form A (Anhydrous)

A sample of Form B (THF solvate) (Example11) was dried under vacuum at RT for 3 days.

### Example 6—Form A (Anhydrous)

A sample of Form B (THF solvate) (Example11) was stored at 40° C./75% RH for 7 days.

### Example 7—Form A (Anhydrous)

A sample of Form D (nitromethane solvate) (Example 37) was dried under vacuum at RT for 3 days.

### Example 8—Form A (Anhydrous)

A sample of Form D (nitromethane solvate) (Example 37) was stored at 40° C./75% RH for 7 days.

### Example 9—Form A (Anhydrous)

A sample of Form E (acetonitrile solvate) (Example 41) was dried under vacuum at RT for 3 days.

### Example 10—Form A (Anhydrous)

A sample of Form E (acetonitrile solvate) (Example 41) was stored at 40° C./75% RH for 7 days.

### Characterisation of Form a (Anhydrous)

FIG. 1 shows a representative XRPD pattern of entrectinib anhydrate (Form A). The following table provides an XRPD peak list for Form A:

| Angle | Intensity % |
|---|---|
| 2-Theta ° | % |
| 7.5 | 66.1 |
| 7.8 | 5.6 |
| 8.3 | 24.4 |
| 11.0 | 2.9 |
| 11.6 | 11.1 |
| 12.2 | 9.8 |
| 13.2 | 6.0 |
| 13.7 | 16.4 |
| 14.9 | 100.0 |
| 15.4 | 4.9 |
| 15.8 | 7.7 |
| 16.5 | 5.2 |
| 17.0 | 11.3 |
| 17.4 | 52.7 |
| 18.1 | 12.8 |
| 18.5 | 7.9 |
| 19.6 | 6.7 |
| 20.1 | 41.7 |
| 21.1 | 15.7 |
| 21.7 | 11.4 |
| 22.1 | 16.5 |
| 22.6 | 23.9 |
| 23.2 | 8.1 |
| 23.7 | 17.5 |
| 24.1 | 12.7 |
| 24.4 | 22.0 |
| 25.0 | 8.6 |
| 26.8 | 7.6 |
| 27.4 | 7.4 |
| 27.8 | 8.0 |
| 28.0 | 7.3 |

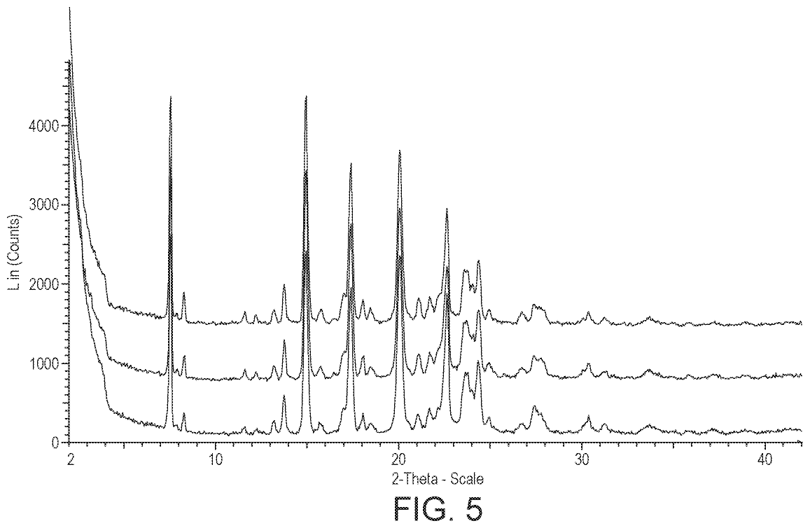

Entrectinib anhydrate (Form A) was also characterised as follows:
TGA and DSC analysis (see FIG. 2).
GVS isotherm analysis (see FIG. 3).
XRPD analysis pre- (bottom) and post- (top) GVS (see FIG. 4).
XRPD analysis after (a) before storage (bottom), (b) after 28 days storage at 40° C./75% RH (relative humidity) (middle); and (c) after 28 days storage 25° C./97% RH (top) (FIG. 5). The XRPD overlays illustrate that entrectinib anhydrate (Form A) is stable under the storage conditions.

The stability, solubility and the IDR of entrectinib anhydrate Form A were determined and the results are shown in the table below:

| | |
|---|---|
| Stability of Form A | Open vial-up to 30 days at 40° C./75% RH |
| | and 25° C./97% RH, no change in XRPD |
| | diffractogram and chemical purity |
| | Closed vial-up to 6 months at 40° C./75% |
| | RH and 25° C./97% RH, no change in |
| | XRPD diffractogram and chemical purity |
| Solubility of Form A | pH 1.2->5.0 mg/ml |
| after 4 hours | pH 2.8->5.0 mg/ml |
| | pH 4.5->5.0 mg/ml |
| | pH 6.8-0.0024 mg/ml |
| | pH 7.5-0.0013 mg/ml |
| IDR | pH 2.8: 2.5 mg/min/cm² |

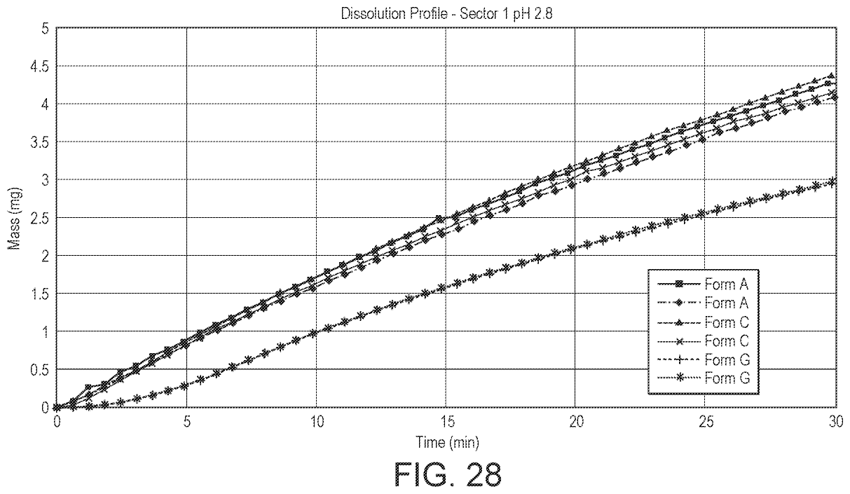

The IDR dissolution profile for anhydrous entrectinib Form A at pH 2.8 is shown in FIG. 28.

### Form B (THF Solvate)

### Example 11—Form B (THF Solvate)

Entrectinib (39.2 mg) was dissolved in THF (200 μl, 5 vol) at 50° C. The solution was left at 50° C. for 1 hour before cooling to 5° C. at 0.1° C./min and left overnight. The resulting suspension was aliquoted for XRPD.

### Example 12—Form B (THF Solvate)

Entrectinib (38.8 mg) was dissolved in THF (200 μl, 5 vol) and left to stir for 5 minutes at 50° C. After 5 minutes of stirring, the sample was treated with water (400 μl). The resulting oil was then left at 50° C. for 1 hour before cooling to 5° C. at 0.1° C./min and left overnight. The resulting suspension was filtered and dried under suction before analysis by XRPD.

### Characterisation of Form B (THF Solvate)

FIG. 6 shows a representative XRPD pattern of entrectinib THF solvate (Form B). The following table provides an XRPD peak list for Form B:

| Angle | Intensity % |
|---|---|
| 2-Theta ° | % |
| 7.1 | 41.3 |
| 7.6 | 59.5 |
| 9.2 | 8.9 |
| 10.0 | 13.2 |
| 10.6 | 44.5 |
| 11.3 | 9.8 |
| 12.2 | 30.5 |
| 12.8 | 13.2 |
| 13.0 | 16.8 |
| 13.3 | 63.7 |
| 13.8 | 8.3 |
| 14.4 | 11.9 |
| 15.0 | 12.5 |
| 16.0 | 39.6 |
| 16.6 | 46.2 |
| 17.1 | 12.3 |
| 17.7 | 35.9 |
| 18.1 | 11.3 |
| 18.5 | 100.0 |
| 18.9 | 16.6 |
| 19.2 | 36.6 |
| 19.4 | 27.3 |
| 19.8 | 26.4 |
| 20.1 | 48.4 |
| 20.6 | 60.5 |
| 21.3 | 27.9 |
| 22.1 | 23.1 |
| 22.6 | 30.9 |
| 23.1 | 19.7 |
| 23.4 | 44.1 |
| 24.4 | 10.3 |
| 24.8 | 26.6 |
| 25.8 | 24.7 |
| 26.2 | 12.3 |
| 26.6 | 13.4 |
| 27.1 | 11.8 |
| 28.5 | 6.4 |
| 29.4 | 4.9 |
| 30.0 | 8.4 |
| 30.1 | 7.0 |
| 30.4 | 7.4 |
| 30.9 | 6.3 |

Entrectinib THF solvate (Form B) was also characterised by TGA and DSC analysis (see FIG. 7).

### Form C (Anhydrous)

### Example 13—Form C (Anhydrous)

Entrectinib (38.9 mg) was dissolved in acetone (1600 μl, 40 vol) at 50° C. The solution was left at 50° C. for 1 hour before cooling to 5° C. at 0.1° C./min and left overnight. The resulting suspension was filtered and dried under suction before analysis by XRPD.

### Example 14—Form C (Anhydrous)

Entrectinib (40.0 mg) was dissolved in MEK (200 μl, 5 vol) at 50° C. The solution was left at 50° C. for 1 hour before cooling to 5° C. at 0.1° C./min and left overnight. The resulting suspension was filtered and dried under suction before analysis by XRPD.

### Example 15—Form C (Anhydrous)

Entrectinib (39.4 mg) was dissolved in MIBK (200 μl, 5 vol) at 50° C. The solution was left at 50° C. for 1 hour before cooling to 5° C. at 0.1° C./min and left overnight. The resulting suspension was filtered and dried under suction before analysis by XRPD.

### Example 16—Form C (Anhydrous)

Entrectinib (39.3 mg) was dissolved in DCM (400 μl, 10 vol) at 50° C. The solution was left at 50° C. for 1 hour before cooling to 5° C. at 0.1° C./min and left overnight. The resulting suspension was filtered and dried under suction before analysis by XRPD.

### Example 17—Form C (Anhydrous)

Entrectinib (39.0 mg) was dissolved in 1,2-dimethoxyethane (400 μl, 10 vol) at 50° C. The solution was left at 50° C. for 1 hour before cooling to 5° C. at 0.1° C./min and left overnight. The resulting suspension was aliquoted for XRPD.

### Example 18—Form C (Anhydrous)

Entrectinib (39.5 mg) was dissolved in acetone/water (95:5 v/v) (800 μl, 20 vol) at 50° C. The solution was left at 50° C. for 1 hour before cooling to 5° C. at 0.1° C./min and left overnight. The resulting suspension was filtered and dried under suction before analysis by XRPD.

### Example 19—Form C (Anhydrous)

Entrectinib (38.8 mg) was dissolved in acetone (4 ml, 100 vol) at 25° C. and the solution was left uncapped to evaporate. Once dry, the solid was analysed by XRPD.

### Example 20—Form C (Anhydrous)

Entrectinib (39.2 mg) was dissolved in DCM (1600 μl, 40 vol) at 25° C. and the solution was left uncapped to evaporate. Once dry, the solid was analysed by XRPD.

### Example 21—Form C (Anhydrous)

Entrectinib (40.1 mg) was dissolved in 1,2-dimethoxyethane (1600 μl, 40 vol) at 25° C. and the solution was left uncapped to evaporate. Once dry, the solid was analysed by XRPD.

### Example 22—Form C (Anhydrous)

Entrectinib (39.9 mg) was dissolved in acetone/water (95:5 v/v) (2400 μl, 60 vol) at 25° C. and the solution was left uncapped to evaporate. Once dry, the solid was analysed by XRPD.

### Example 23—Form C (Anhydrous)

Entrectinib (39.0 mg) was treated with MEK (200 μl, 5 vol) and left to stir for 5 minutes at 50° C. After 5 minutes of stirring, the sample was treated with isopropyl acetate (200 μl). The resulting suspension was then left at 50° C. for 1 hour before cooling to 5° C. at 0.1° C./min and left overnight. The resulting suspension was filtered and dried under suction before analysis by XRPD.

### Example 24—Form C (Anhydrous)

Entrectinib (38.6 mg) was dissolved in THF (200 μl, 5 vol) and left to stir for 5 minutes at 50° C. After 5 minutes of stirring, the sample was treated with isopropyl acetate (1000 μl). The resulting solution was then left at 50° C. for 1 hour before cooling to 5° C. at 0.1° C./min and left overnight. The resulting suspension was filtered and dried under suction before analysis by XRPD.

### Example 25—Form C (Anhydrous)

Entrectinib (97.5 mg) was dissolved in DCM (1 ml, 10 vol) at 50° C. The solution was left at 50° C. for 1 hour before cooling to 5° C. at 0.1° C./min. The sample was left at 5° C. for 3 days. The resulting suspension was filtered and dried under suction before analysis by XRPD.

### Example 26—Form C (Anhydrous)

A sample of Form F (isopropyl acetate solvate) (Example 46) was dried under vacuum at RT for 3 days.

### Example 27—Form C (Anhydrous)

A sample of Form F (isopropyl acetate solvate) (Example 46) was stored at 40° C./75% RH for 7 days.

### Example 28—Form C (Anhydrous)

Entrectinib (38.9 mg) was treated with ethyl acetate (200 μl, 5 vol) and left to stir at 60° C. for 3 days. The resulting suspension was filtered and dried under suction before analysis by XRPD.

### Example 29—Form C (Anhydrous)

Entrectinib (38.9 mg) was treated with 1,2-dimethoxyethanol (200 μl, 5 vol) and left to stir at 60° C. for 3 days. The resulting suspension was filtered and dried under suction before analysis by XRPD.

### Example 30—Form C (Anhydrous)

Entrectinib (1 g) was dissolved in DCM (10 ml, 10 vol) at 50° C. The solution was then seeded with Form C (Example 25) then left at 50° C. for 1 hour before cooling to 5° C. at 0.1° C./min and left overnight.

The resulting suspension was aliquoted for XRPD.

### Characterisation of Form C (Anhydrous)

FIG. 8 is a representative XRPD pattern of entrectinib anhydrate (Form C). The following table provides an XRPD peak list for Form C:

| Angle | Intensity % |
|---|---|
| 2-Theta ° | % |
| 8.0 | 20.7 |
| 8.8 | 10.7 |
| 9.4 | 27.2 |
| 10.5 | 14.3 |
| 13.1 | 24.6 |
| 14.0 | 23.7 |
| 14.5 | 35.8 |
| 15.2 | 22.4 |
| 15.4 | 33.6 |
| 16.0 | 41.7 |
| 16.2 | 31.2 |
| 17.1 | 13.1 |
| 17.7 | 36.2 |
| 19.1 | 16.2 |
| 19.6 | 22.6 |
| 20.7 | 100.0 |
| 21.3 | 14.9 |
| 21.6 | 26.8 |
| 22.4 | 49.9 |
| 22.7 | 100.0 |
| 23.1 | 20.4 |
| 23.5 | 30.5 |
| 24.1 | 39.7 |
| 25.2 | 10.9 |
| 25.9 | 6.2 |
| 26.4 | 5.5 |
| 27.3 | 6.3 |
| 27.7 | 10.5 |
| 28.0 | 33.8 |
| 28.3 | 21.2 |
| 28.6 | 7.8 |
| 29.2 | 7.2 |
| 29.5 | 8.9 |
| 29.9 | 5.9 |

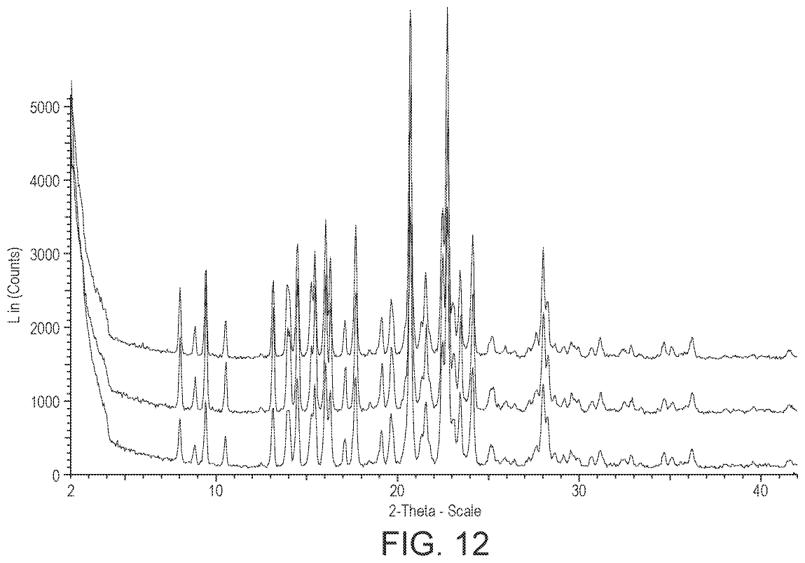

Entrectinib anhydrate (Form C) was also characterised as follows:
TGA and DSC analysis (see FIG. 9).
GVS isotherm analysis (see FIG. 10).
XRPD analysis before (bottom) and after (top) the GVS experiment (see FIG. 11).
XRPD analysis after (a) before storage (bottom), (b) after 28 days storage at 40° C./75% RH (middle); and (c) after 28 days storage 25° C./97% RH (top) (FIG. 12). The XRPD overlays illustrate that entrectinib anhydrate (Form C) is stable under the storage conditions (see FIG. 12).

The stability, solubility and the IDR of entrectinib anhydrate Form C were determined and the results are shown in the table below:

| | |
|---|---|
| Stability of Form C | Open vial-up to 4 weeks at 40° C./75% RH |
| | and 25° C./97% RH, no change in XRPD |
| | diffractogram and chemical purity |
| | Closed vial-up to 6 months at 40° C./75% |
| | RH and 25° C./97% RH, no change in |
| | XRPD diffractogram and chemical purity |
| Solubility of Form | pH 1.2-4.3 mg/ml |
| C after 4 hours | pH 2.8->5.0 mg/ml |
| | pH 4.5-4.0 mg/ml |
| | pH 6.8-0.0013 mg/ml |
| | pH 7.5-0.0006 mg/ml |
| IDR | pH 2.8: 2.5 mg/min/cm² |

The IDR dissolution profile for anhydrous entrectinib Form C at pH 2.8 is shown in FIG. 28.

### Form D (Nitromethane Solvate)

### Example 31—Form D (Nitromethane Solvate)

Entrectinib (39.1 mg) was treated with nitromethane (4 ml, 100 vol) and heated to 50° C. before being placed into maturation (RT/50° C., cycles of 4 hours at each temperature) for 6 days. The resulting suspension was filtered and dried under suction before analysis by XRPD.

### Example 32—Form D (Nitromethane Solvate)

Entrectinib (37.3 mg) was treated with MEK (200 μl, 5 vol) and left to stir for 5 minutes at 50° C. After 5 minutes of stirring, the sample was treated with nitromethane (200 μl). The resulting suspension was then left at 50° C. for 1 hour before cooling to 5° C. at 0.1° C./min and left overnight. The resulting suspension was filtered and dried under suction before analysis by XRPD.

### Example 33—Form D (Nitromethane Solvate)

Entrectinib (39.2 mg) was dissolved in THF (200 μl, 5 vol) and left to stir for 5 minutes at 50° C. After 5 minutes of stirring, the sample was treated with nitromethane (1000 μl). The resulting solution was then left at 50° C. for 1 hour before cooling to 5° C. at 0.1° C./min and left overnight. The resulting suspension was filtered and dried under suction before analysis by XRPD.

### Example 34—Form D (Nitromethane Solvate)

Entrectinib (38.6 mg) was treated with toluene (200 μl, 5 vol) and left to stir for 5 minutes at 50° C. After 5 minutes of stirring, the sample was treated with nitromethane (200 μl). The resulting suspension was then left at 50° C. for 1 hour before cooling to 5° C. at 0.1° C./min and left overnight. The resulting suspension was filtered and dried under suction before analysis by XRPD.

### Example 35—Form D (Nitromethane Solvate)

Entrectinib (38.4 mg) was dissolved in DMF (200 μl, 5 vol) and left to stir for 5 minutes at 50° C. After 5 minutes of stirring, the sample was treated with nitromethane (1000 μl). The resulting solution was then left at 50° C. for 1 hour before cooling to 5° C. at 0.1° C./min and left overnight. The resulting suspension was filtered and dried under suction before analysis by XRPD.

### Example 36—Form D (Nitromethane Solvate)

Entrectinib (38.1 mg) was dissolved in 1-propanol (200 μl, 5 vol) and left to stir for 5 minutes at 50° C. After 5 minutes of stirring, the sample was treated with nitromethane (1000 μl). The resulting solution was then left at 50° C. for 1 hour before cooling to 5° C. at 0.1° C./min and left overnight. The resulting suspension was filtered and dried under suction before analysis by XRPD.

### Example 37—Form D (Nitromethane Solvate)

Entrectinib (98.7 mg) was treated with MEK (500 μl, 5 vol) and left to stir for 5 minutes at 50° C. After 5 minutes of stirring, the sample was treated with nitromethane (500 μl). The resulting suspension was then left at 50° C. for 1 hour before cooling to 5° C. at 0.1° C./min. The sample was left at 5° C. for 3 days. The resulting suspension was filtered and dried under suction before analysis by XRPD.

### Example 38—Form D (Nitromethane Solvate)

Entrectinib (39.5 mg) was treated with nitromethane (200 μl, 5 vol) and left to stir at 60° C. for 3 days. The resulting suspension was filtered and dried under suction before analysis by XRPD.

### Characterisation of Form D (Nitromethane Solvate)

FIG. 13 is a representative XRPD pattern of entrectinib nitromethane solvate (Form D). The following table provides an XRPD peak list for Form D:

| Angle | Intensity % |
|---|---|
| 2-Theta ° | % |
| 7.2 | 31.6 |
| 7.5 | 33.8 |
| 7.7 | 41.8 |
| 10.2 | 12.2 |
| 10.8 | 17.5 |
| 12.3 | 22.9 |
| 12.9 | 15.0 |
| 13.5 | 100.0 |
| 14.0 | 7.8 |
| 14.5 | 17.2 |
| 15.0 | 19.0 |
| 16.1 | 32.4 |
| 16.8 | 51.4 |
| 17.6 | 54.1 |
| 18.0 | 10.6 |
| 18.6 | 24.6 |
| 19.0 | 20.3 |
| 19.6 | 46.3 |
| 19.8 | 51.0 |
| 20.4 | 63.1 |
| 20.6 | 33.0 |
| 20.9 | 41.0 |
| 21.3 | 25.0 |
| 21.5 | 16.1 |
| 22.3 | 59.7 |
| 22.6 | 39.3 |
| 22.9 | 41.0 |
| 23.2 | 51.5 |
| 23.7 | 27.4 |
| 23.9 | 13.5 |
| 24.7 | 36.8 |
| 24.9 | 18.6 |
| 25.1 | 11.8 |
| 26.0 | 46.9 |
| 26.6 | 17.4 |
| 27.2 | 31.2 |
| 28.4 | 10.8 |
| 30.4 | 24.0 |

Entrectinib nitromethane solvate (Form D) was also characterised as follows:
TGA and DSC analysis (see FIG. 14).

### Form E (Acetonitrile Solvate)

### Example 39—Form E (Acetonitrile Solvate)

Entrectinib (39.0 mg) was dissolved in THF (200 μl, 5 vol) and left to stir for 5 minutes at 50° C. After 5 minutes of stirring, the sample was treated with acetonitrile (1000 μl). The resulting solution was then left at 50° C. for 1 hour before cooling to 5° C. at 0.1° C./min and left overnight. The resulting suspension was filtered and dried under suction before analysis by XRPD.

### Example 40—Form E (Acetonitrile Solvate)

Entrectinib (38.6 mg) was dissolved in DMF (200 μl, 5 vol) and left to stir for 5 minutes at 50° C. After 5 minutes of stirring, the sample was treated with acetonitrile (1000 μl). The resulting solution was then left at 50° C. for 1 hour before cooling to 5° C. at 0.1° C./min and left overnight. The resulting suspension was filtered and dried under suction before analysis by XRPD.

### Example 41—Form E (Acetonitrile Solvate)

Entrectinib (97.7 mg) was treated with MEK (500 μl, 5 vol) and left to stir for 5 minutes at 50° C. After 5 minutes of stirring, the sample was treated with acetonitrile (1000 μl). The resulting suspension was then left at 50° C. for 1 hour before cooling to 5° C. at 0.1° C./min. The sample was left at 5° C. for 3 days. The resulting suspension was filtered and dried under suction before analysis by XRPD.

### Example 42—Form E (Acetonitrile Solvate)

Entrectinib (40.2 mg) was treated with acetonitrile (200 μl, 5 vol) and left to stir at 60° C. for 3 days. The resulting suspension was filtered and dried under suction before analysis by XRPD.

### Example 43—Form E (Acetonitrile Solvate)

Entrectinib (1.0 g) was dissolved in 1-propanol (5 ml, 5 vol) at 50° C. The solution was treated with acetonitrile (5×5 ml), seeded with Form A (Entrectinib) and then left at 50° C. for 1 hour before cooling to 5° C. at 0.1° C./min and left overnight. The resulting suspension was filtered at 5° C.

### Example 44—Form E (Acetonitrile Solvate)

Entrectinib (4.9 g) was treated with 1-propanol at 70° C., 1 vol (5 ml) at a time until dissolution. At 2 vol the sample when into solution and then precipitated, further solvent added up to 5 vol (total 25 ml). The sample remained as a suspension, so heated to 90° C. to dissolve solid. Once the sample was a clear solution, the sample was cooled at 0.3° C./min. At −50° C. the sample was a suspension again, so reheated to 90° C. to dissolve solids again.

The clear solution was then cooled to 70° C. and acetonitrile (100 ml) added via a dropping funnel. During anti-solvent addition, once the sample was slightly cloudy it was seeded with Form A. After anti-solvent addition was complete, the sample was cooled to 5° C. at 0.3° C./min. The suspension at 5° C. was filtered.

### Characterisation of Form E (Acetonitrile Solvate)

FIG. 15 is a representative XRPD pattern of entrectinib acetonitrile solvate (Form E). The following table provides an XRPD peak list for Form E:

| Angle | Intensity % |
|---|---|
| 2-Theta ° | % |
| 7.1 | 19.6 |
| 7.5 | 100.0 |
| 7.8 | 30.1 |
| 8.3 | 23.3 |
| 10.8 | 17.3 |
| 11.6 | 19.7 |
| 12.2 | 15.2 |
| 12.4 | 14.8 |
| 13.0 | 18.0 |
| 13.6 | 77.0 |
| 15.0 | 73.1 |
| 15.9 | 18.3 |
| 16.4 | 20.3 |
| 16.6 | 21.8 |
| 17.0 | 23.5 |
| 17.4 | 77.5 |
| 18.0 | 39.0 |
| 18.4 | 24.4 |
| 18.6 | 30.7 |
| 19.1 | 27.6 |
| 19.6 | 49.8 |
| 20.2 | 77.9 |
| 20.6 | 35.5 |
| 20.9 | 32.6 |
| 21.1 | 32.5 |
| 21.3 | 30.6 |
| 21.8 | 24.5 |
| 22.0 | 32.4 |
| 22.7 | 68.2 |
| 23.7 | 40.3 |
| 24.4 | 44.1 |
| 25.0 | 30.4 |
| 25.7 | 19.7 |
| 26.0 | 31.2 |
| 26.5 | 28.9 |
| 26.9 | 25.8 |
| 27.4 | 22.7 |
| 27.8 | 19.4 |

Entrectinib acetonitrile solvate (Form E) was also characterised as follows:
TGA and DSC analysis (see FIG. 16).

### Form F (Isopropyl Acetate Solvate)

### Example 45—Form F (Isopropyl Acetate Solvate)

Entrectinib (38.3 mg) was dissolved in 1-propanol (200 μl, 5 vol) and left to stir for 5 minutes at 50° C. After 5 minutes of stirring, the sample was treated with isopropyl acetate (1000 μl). The resulting solution was then left at 50° C. for 1 hour before cooling to 5° C. at 0.1° C./min and left overnight. The resulting suspension was filtered and dried under suction before analysis by XRPD.

### Example 46—Form F (Isopropyl Acetate Solvate)

Entrectinib (97.5 mg) was dissolved in IPA (500 μl, 5 vol) and left to stir for 5 minutes at 50° C. After 5 minutes of stirring, the sample was treated with isopropyl acetate (2.5 ml). The resulting solution was then left at 50° C. for 1 hour before cooling to 5° C. at 0.1° C./min and left overnight. The resulting suspension was filtered and dried under suction before analysis by XRPD.

### Characterisation of Form F (Isopropyl Acetate Solvate)

FIG. 17 is a representative XRPD pattern of entrectinib isopropyl acetate solvate (Form F). The following table provides an XRPD peak list for Form F:

| Angle | Intensity % |
|---|---|
| 2-Theta ° | % |
| 6.5 | 17.7 |
| 7.6 | 38.7 |
| 8.0 | 12.2 |
| 8.9 | 28.4 |
| 9.4 | 29.5 |
| 10.5 | 15.2 |
| 10.8 | 21.0 |
| 11.7 | 13.9 |
| 12.9 | 43.8 |
| 13.1 | 26.2 |
| 14.0 | 16.8 |
| 14.5 | 24.7 |
| 15.2 | 30.1 |
| 15.4 | 24.6 |
| 16.1 | 17.2 |
| 16.3 | 20.5 |
| 16.6 | 17.6 |
| 17.0 | 15.7 |
| 17.7 | 100.0 |
| 18.4 | 52.2 |
| 18.7 | 26.5 |
| 18.9 | 24.3 |
| 19.1 | 20.2 |
| 19.6 | 25.5 |
| 20.4 | 34.8 |
| 20.7 | 22.3 |
| 21.8 | 37.0 |
| 22.4 | 32.8 |
| 22.9 | 43.3 |
| 23.5 | 29.1 |
| 24.7 | 16.5 |
| 25.1 | 24.5 |
| 25.7 | 15.1 |
| 26.1 | 14.7 |
| 28.2 | 18.5 |
| 30.8 | 10.6 |

Entrectinib isopropyl acetate solvate (Form F) was also characterised as follows:
TGA and DSC analysis (see FIG. 18).

### Form G (Monohydrate)

### Example 47—Form G (Monohydrate)

Entrectinib (38.6 mg) was dissolved in 1-propanol (200 μl, 5 vol) and left to stir for 5 minutes at 50° C. After 5 minutes of stirring, the sample was treated with water (400 μl). The resulting suspension was then left at 50° C. for 1 hour before cooling to 5° C. at 0.1° C./min and left overnight. The resulting suspension was filtered and dried under suction before analysis by XRPD.

### Example 48—Form G (Monohydrate)

Entrectinib (1.0 g) was dissolved in 1-propanol (5 ml, 5 vol) at 50° C. The solution was treated with water (10 ml) and then seeded with Form G (Example 47). The resulting suspension was then left at 50° C. for 1 hour before cooling to 5° C. at 0.1° C./min and left overnight. The resulting suspension was filtered and dried under vacuum at RT overnight.

### Example 49—Form G (Monohydrate)

Entrectinib (5.1 g) was treated with 1-propanol at 70° C., 1 vol (5 ml) at a time until dissolution. At 3 vol the sample when into solution and then precipitated, further solvent added up to 5 vol (total 25 ml). The sample remained as a suspension, so heated to 90° C. to dissolve solid. Once the sample was a clear solution, the sample was cooled at 0.3° C./min. At −50° C. the sample was a suspension again, so reheated to 90° C. to dissolve solids again.

The clear solution was then cooled to 70° C. and water (50 ml) added via a dropping funnel. During anti-solvent addition, once the sample was slightly cloudy it was seeded with Form G (Example 48). After anti-solvent addition was complete, the sample was cooled to 5° C. at 0.3° C./min. The suspension at 5° C. was filtered and dried under vacuum at RT for 1 day.

### Example 50—Form G (Monohydrate)

### Preparation of Amorphous Entrectinib

Entrectinib (1.2 g) was dissolved in THF/water (7:3 v/v) (6 ml, 5 volumes) and sonicated to yield a solution. The solution was filtered through a 0.45 μm nylon membrane disc filter. The solution was dispensed to HPLC vials in 200 μl aliquots (approx. 40 mg entrectinib). These were flash frozen in a dry ice/acetone bath and the frozen samples were lyophilised using a freeze dryer overnight to form amorphous entrectinib. A single HPLC was analysed as a representative sample for the batch.

### Preparation of Entrectinib Form G

Amorphous entrectinib (approx. 40 mg) was suspended in the relevant solvent (see the table below for solvent and quantity). The samples were stirred at room temperature for 3 days. An aliquot of the suspensions/slurries were analysed by XRPD after transferring material directly to the sample holder with a spatula.

| Example | Solvent/water (v/v) | XRPD |
|---|---|---|
| 50.1 | 1-PrOH (9:1), 100 μl | Form G |
| 50.2 | 1-PrOH (8:2), 100 μl | Form G |
| 50.3 | isopropanol (8:2), 100 μl | Form G |

### Example 51—Form G (Monohydrate)

Entrectinib (100 mg) was dispensed and suspended in 1-PrOH (0.75 ml, 7.5 volumes) at 70° C. with stirring (400 rpm) for approx. 10 minutes. A turbid solution was observed and water (20 μl, approx. 2.6 vol %) was added resulting in a clear solution which was stirred for approx. 10 minutes. To the solution, water (0.77 ml, 1 equivalent volume) was added dropwise over approx. 5 minutes. The resulting solution was cooled to 50° C. at 0.3° C./minute.

At 50° C., the solution was seeded with entrectinib Form G as prepared in Example 50.1 (approx. 3-5 mg). The seeds persisted and water (0.77 ml, 1 equivalent volume) was added dropwise over approx. 5 minutes. A turbid solution with some solids near the bottom of the vessel were observed. The sample was cooled to 5° C. at 0.3° C./minute and stirred at 5° C. overnight. An aliquot was analysed by XRPD and the bulk suspension was filtered by Buchner filtration under vacuum (55 mm Grade 1 Whatman filter) to yield entrectinib Form G. Yield: 73 mg (71%)

### Example 52—Form G (Monohydrate)

Entrectinib (1 g) was dispensed and dissolved in 1-PrOH/water (97.5:2.5 v/v) (6 ml, 6 volumes) at 70° C. with stirring (400 rpm) for approx. 20 minutes. Traces of solid were noted above the solvent line on vessel walls and added back to the solvent to dissolve. To the solution, water (6 ml, 1 equivalent volume) was added dropwise over 10 minutes. The solution was stirred for approx. 5 minutes during which the solution appeared to become seeded with solid from further up the vessel wall. The sample was cooled to 50° C. at 0.3° C./minute.

At 50° C. the light suspension was seeded with entrectinib Form G as prepared in Example 51 (50 mg, 5 wt %). The seeds persisted and water (6 ml, 1 equivalent volume) was added dropwise over approx. 10 minutes. A suspension was noted. The sample was cooled to 5° C. at 0.3° C./minute and stirred at 5° C. for 4 days. The bulk suspension was filtered by Buchner filtration under vacuum (55 mm Grade 1 Whatman filter), dried under vacuum for 15 minutes to yield entrectinib Form G as determined by XRPD.

### Example 53—Form G (Monohydrate)

Two separate crystallisations were run in tandem; Example 53.1 and 53.2 Entrectinib (1.0 g) was dispensed and dissolved in 1-PrOH/water (9:1 v/v) (7.5 ml, 7.5 volumes) at 50° C. with stirring (400 rpm) for approx. 15 minutes. To the solution, water (7.5 ml, 1 equivalent volume) was added dropwise over 10 minutes. The solution, which may be turbid, was seeded with Form G as prepared in Example 52 (50 mg, 5 wt %). The seeds persisted and water (7.5 ml, 1 equivalent volume) was added dropwise over approx. 10 minutes. A suspension resulted. The sample was cooled to 5° C. at 0.3° C./minute and stirred at 5° C. for 5 days. The bulk suspension was filtered by Buchner filtration under vacuum (55 mm Grade 1 Whatman filter), dried under vacuum for 15 minutes to yield entrectinib Form G as determined by XRPD.

Yield: Example 53.1-0.94 g (92%)
Example 53.2-0.96 g (93%)

### Characterisation of Form G (Entrectinib Monohydrate)

FIG. 19 is a representative XRPD pattern of entrectinib monohydrate (Form G). The following table provides an XRPD peak list for Form G:

| Angle | Intensity % |
|---|---|
| 2-Theta ° | % |
| 7.6 | 5.1 |
| 7.8 | 9.7 |
| 9.0 | 48.9 |
| 9.8 | 4.3 |
| 10.2 | 8.4 |
| 10.6 | 30.2 |
| 13.4 | 5.3 |
| 14.3 | 5.2 |
| 15.0 | 9.0 |
| 15.4 | 3.2 |
| 15.8 | 19.1 |
| 16.0 | 30.4 |
| 16.5 | 11.8 |
| 16.7 | 31.4 |
| 17.1 | 74.7 |
| 17.4 | 27.2 |
| 17.7 | 34.1 |
| 18.0 | 22.4 |
| 18.4 | 22.7 |
| 19.1 | 13.2 |
| 19.3 | 45.8 |
| 19.5 | 6.6 |
| 19.8 | 6.5 |
| 20.2 | 9.1 |
| 20.6 | 5.1 |
| 20.8 | 8.0 |
| 21.3 | 9.8 |
| 21.7 | 34.7 |
| 21.8 | 44.7 |
| 22.3 | 25.6 |
| 22.8 | 100.0 |
| 23.3 | 4.8 |
| 23.6 | 5.3 |
| 24.0 | 6.0 |
| 24.5 | 15.1 |
| 24.8 | 70.8 |
| 25.3 | 33.0 |
| 25.6 | 4.1 |
| 26.0 | 4.0 |
| 26.2 | 4.4 |
| 26.4 | 6.3 |
| 26.9 | 11.4 |
| 27.3 | 9.5 |
| 27.5 | 7.3 |
| 28.2 | 3.2 |
| 28.4 | 4.3 |
| 28.6 | 10.4 |
| 28.9 | 14.0 |
| 29.0 | 6.5 |
| 29.6 | 10.4 |
| 30.3 | 14.4 |

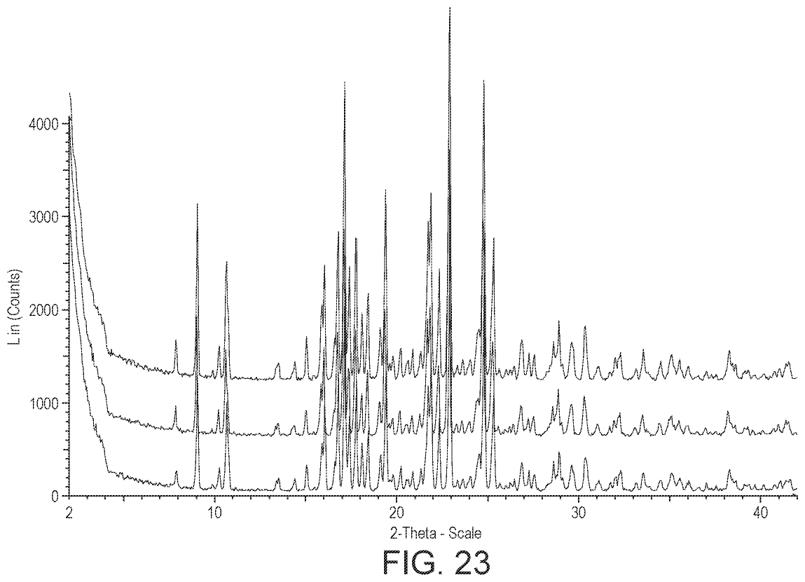

Entrectinib monohydrate (Form G) was also characterised as follows:
TGA and DSC analysis (see FIG. 20).
GVS isotherm analysis (see FIG. 21).
XRPD analysis pre- (bottom) and post- (top) GVS (see FIG. 22).
XRPD analysis after (a) before storage (bottom), (b) after 28 days storage at 40° C./75% RH (relative humidity) (middle); and (c) after 28 days storage 25° C./97% RH (top) (FIG. 23). The XRPD overlays illustrate that entrectinib monohydrate (Form G) is stable under the storage conditions.

The stability, solubility and the IDR of entrectinib monohydrate Form G were determined and the results are shown in the table below:

| | |
|---|---|
| Stability of Form G | Open vial-up to 4 weeks at 40° C./75% RH |
| | and 25° C./97% RH, no change in XRPD |
| | diffractogram and chemical purity |
| | Closed vial-up to 6 months at 40° C./75% |
| | RH and 25° C./97% RH, no change in |
| | XRPD diffractogram and chemical purity |
| Solubility of Form G | pH 1.2->5.0 mg/ml |
| after 4 hours | pH 2.8->5.0 mg/ml |
| | pH 4.5-0.10 mg/ml |
| | pH 6.8-0.0002 mg/ml |
| | pH 7.5-0.0002 mg/ml |
| IDR | pH 2.8: 1.6 mg/min/cm² |

The IDR dissolution profile for entrectinib monohydrate Form G at pH 2.8 is shown in FIG. 28.

### Form H (2-Methyl THF Solvate 0.5 eq)

### Example 54—Form H

Entrectinib (39.6 mg) was dissolved in 2-methyl THF (200 μl, 5 vol) at 25° C. and the solution was left uncapped to evaporate. Once dry, the solid was analysed by XRPD.

### Example 55—Form H

Entrectinib (97.5 mg) was dissolved in 2-methyl THF (200 μl, 5 vol) at 50° C. The solution was left at 50° C. for 1 hour before cooling to 5° C. at 0.1° C./min. The sample was left at 5° C. for 3 days. The resulting suspension was filtered and dried under suction before analysis by XRPD.

### Example 56—Form H

Entrectinib (98.1 mg) was dissolved in 2-methyl THF (500 μl, 5 vol) at 25° C. and the solution was left uncapped to evaporate. Once dry, the solid was analysed by XRPD.

### Characterisation of Form H (Entrectinib 2-Methyl THF Solvate 0.5 Eq.)

FIG. 24 is a representative XRPD pattern of entrectinib 2-methyl THF solvate 0.5 eq. (Form H). The following table provides an XRPD peak list for Form H:

| Angle | Intensity % |
|---|---|
| 2-Theta ° | % |
| 7.0 | 14.3 |
| 8.2 | 19.5 |
| 9.4 | 38.3 |
| 10.2 | 15.5 |
| 10.4 | 19.8 |
| 11.2 | 15.0 |
| 13.7 | 34.4 |
| 14.5 | 58.6 |
| 16.0 | 11.2 |
| 16.6 | 20.8 |
| 17.1 | 13.6 |
| 17.5 | 100.0 |
| 17.8 | 12.6 |
| 18.2 | 15.5 |
| 18.5 | 12.2 |
| 18.9 | 19.8 |
| 19.2 | 40.3 |
| 19.8 | 35.0 |
| 20.3 | 17.6 |
| 21.3 | 10.1 |
| 21.9 | 22.5 |
| 22.3 | 23.2 |
| 22.5 | 25.7 |
| 23.0 | 20.2 |
| 23.4 | 19.1 |
| 23.8 | 8.1 |
| 24.7 | 8.0 |
| 25.2 | 12.8 |
| 26.5 | 8.0 |
| 27.3 | 5.8 |
| 29.0 | 5.6 |
| 30.0 | 9.7 |
| 30.3 | 9.1 |

Entrectinib 2-methyl THF solvate 0.5 eq. (Form H) was also characterised as follows:
TGA and DSC analysis (see FIG. 25).

### Form J (Methanol Solvate 1 ea)

### Example 57—Form J

Entrectinib (39.5 mg) was treated with methanol (4 ml, 100 vol) and heated to 50° C. before being placed into maturation (RT/50° C., cycles of 4 hours at each temperature) for 6 days. The resulting suspension was filtered and dried under suction before analysis by XRPD.

### Example 58—Form J

Entrectinib (40.3 mg) was treated with methanol (200 μl, 5 vol) and left to stir at 60° C. for 3 days. The resulting suspension was filtered and dried under suction before analysis by XRPD.

### Characterisation of Form J (Entrectinib Methanol Solvate 1 Eq.)

FIG. 26 is a representative XRPD pattern of entrectinib methanol solvate 1 eq. (Form J). The following table provides an XRPD peak list for Form J:

| Angle | Intensity % |
|---|---|
| 2-Theta ° | % |
| 7.8 | 15.6 |
| 8.9 | 48.8 |
| 10.3 | 28.6 |
| 10.5 | 13.0 |
| 14.1 | 5.9 |
| 14.5 | 5.3 |
| 14.8 | 10.9 |
| 15.8 | 41.8 |
| 16.3 | 20.2 |
| 16.6 | 60.7 |
| 16.8 | 80.3 |
| 17.1 | 100.0 |
| 17.7 | 38.9 |
| 17.9 | 43.7 |
| 18.2 | 37.7 |
| 18.7 | 26.1 |
| 19.0 | 36.0 |
| 19.5 | 6.0 |
| 19.9 | 16.2 |
| 20.6 | 25.2 |
| 20.9 | 11.0 |
| 21.3 | 75.8 |
| 21.6 | 15.7 |
| 22.4 | 33.0 |
| 22.7 | 88.6 |
| 22.9 | 40.7 |
| 23.6 | 4.8 |
| 23.9 | 14.5 |
| 24.2 | 22.4 |
| 24.8 | 90.9 |
| 25.2 | 9.9 |
| 25.5 | 46.9 |
| 25.8 | 13.0 |
| 25.9 | 14.2 |
| 26.6 | 28.9 |
| 27.1 | 28.9 |
| 28.1 | 17.9 |
| 28.4 | 16.1 |
| 28.6 | 7.9 |
| 29.0 | 8.4 |
| 29.8 | 18.4 |
| 30.2 | 9.8 |

Entrectinib methanol solvate 1 eq. (Form J) was also characterised as follows:
TGA and DSC analysis (see FIG. 27).

1. A process for preparing crystalline entrectinib anhydrate Form A, the process comprising the steps of:
(a) dissolving entrectinib in a first solvent selected from the group consisting of: methyl ethyl ketone (MEK), 1-propanol, 2-propanol and combinations thereof;
(b) adding acetonitrile to the solution of entrectinib; and
(c) recovering entrectinib anhydrate Form A as a crystalline solid.

2. A process for preparing crystalline entrectinib anhydrate Form A, the process comprising the step of exposing a crystalline entrectinib solvate to water vapour,
wherein the crystalline entrectinib solvate is selected from the group consisting of entrectinib acetonitrile solvate Form E, entrectinib THF solvate Form B, and entrectinib nitromethane solvate Form D.

3. A process for preparing crystalline entrectinib anhydrate Form A, the process comprising the step of treating a crystalline entrectinib solvate under vacuum,
wherein the crystalline entrectinib solvate is selected from the group consisting of entrectinib acetonitrile solvate Form E, entrectinib THF solvate Form B, and entrectinib nitromethane solvate Form D.

4. A crystalline form of entrectinib which is crystalline entrectinib anhydrate Form C, having an X-ray powder diffraction pattern comprising one or more peaks selected from the group consisting of about 8.0, 8.8, 9.4, 10.5, 13.1, 14.0, 14.5, 15.2, 15.4, 16.0, 16.2, 17.1, 17.7, 19.1, 19.6, 20.7, 21.3, 21.6, 22.4, 22.7, 23.1, 23.5, 24.1, 25.2, 25.9, 26.4, 27.3, 27.7, 28.0, 28.3, 28.6, 29.2, 29.5, and 29.9 degrees two-theta ±0.2 degrees two-theta.

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. A pharmaceutical composition comprising entrectinib and a pharmaceutically acceptable excipient, wherein the entrectinib is selected from the group consisting of: (i) crystalline entrectinib anhydrate Form A, (ii) crystalline entrectinib anhydrate Form C, and (iii) crystalline entrectinib monohydrate Form G.

29. A method for treating neuroblastoma in a patient comprising administering a therapeutically effective amount of entrectinib to the patient, wherein the entrectinib is selected from the group consisting of: (i) crystalline entrectinib anhydrate Form A, (ii) crystalline entrectinib anhydrate Form C, and (iii) crystalline entrectinib monohydrate Form G.

30. (canceled)

31. (canceled)

32. (canceled)
